SEQUENCE LISTING (51 sequences)

SEQ ID NO: 1 - DNA, 384 bp, Hepatitis C virus H strain
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcugcaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 2 - DNA, 168 bp, Hepatitis C virus H strain
  variation (55)...(67): poly (U)/poly(UC) region
cuccccaaccgaugaagguugggguaaacacuccggccucuuaggccauuuccuguuuuuuuuuucuaaugguggcuccaucuuagcccuagucacggcuagcugugaaagguccgugagccgcaugacugcagagagugcugauacuggccucucugcagaucaugu

SEQ ID NO: 3 - DNA, 573 bp, Hepatitis C virus type I/1a
atgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgataccctcacgtgcggcttcgccgacctcatggggtacataccgctcgtcggcgcccctctcggaggcgctgccagggccctggcgcatggcgtccgggttctggaagacggcgtgaactatgcaacagggaaccttcctggttgctctttctctatcttccttctggccctgctctcttgcctgactgtgcccgcttcagcc

SEQ ID NO: 4 - DNA, 573 bp, Hepatitis C virus type II/1b
atgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccggcccgagggcagggcctgggctcagcccgggtacccttggcccctctatggcaatgagggcatggggtgggcaggatggctcctgtcaccccgcggctctcggcctagttggggccccacggacccccggcgtaggtcgcgtaatttgggtaaggtcatcgataccctcacatgcggcttcgccgacctcatggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtttgaccatcccagcttccgct

SEQ ID NO: 5 - DNA, 573 bp, Hepatitis C virus type III/2A
atgagcacaaatcctaaacctcaaagaaaaaccaaaagaaacactaaccgtcgcccacaagacgttaagtttccgggcggcggccagatcgttggcggagtatacttgttgccgcgcaggggccccaggttgggtgtgcgcgcgacaaggaagacttcggagcggtcccagccacgtgggaggcgccagcccatccccaaagatcggcgctccactggcaagtcctggggaaaaccaggatacccctggcccctatatgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccccgtcgtaggcgccccgcttggtggcgtcgccagagctctcgcgcatggcgtgagagtcctggaggacggggttaattatgcaacagggaacttacctggttgctccttttctatcttcttgctggccctactgtcctgcatcaccattccggtctctgct

SEQ ID NO: 6 - DNA, 573 bp, Hepatitis C virus type IV/2b
atgagcacaaatcctaaacctcaaagaaaaaccaaaagaaacacaaaccgccgcccacaggacgttaagttcccgggtggcggccagatcgttggcggagtttacttgctgccgcgcaggggccccaggttgggtgtgcgcgcgacaaggaagacttccgagcgatcccagccgcgtgggagacgccagcccatcccgaaagatcggcgctccaccggcaagtcctggggaaagccaggatatccttggcccctgtatggaaacgagggctgcggctgggcaggttggctcctgtccccccgcgggtctcgtcctacttggggccccactgacccccggcatagatcacgcaatttgggcaaagtcatcgacaccattacgtgtggtttcgccgacctcatggggtacatccctgtcgttggcgccccggtcggaggcgtcgccagagctctggcacacggtgttagggtcctggaagacgggataaattacgcaacagggaatctgcctggttgctctttttctatcttcttacttgctcttctgtcgtgcttcacagtgccagtgtctgcg

SEQ ID NO: 7 - DNA, 573 bp, Hepatitis C virus type 2c
atgagcacaaatcctaaacctcaaagaaaaaccaaaagaaacactaaccgccgcccacaggacgtcaagttcccgggcggtggccagatcgttggcggagtatacttgctgccgcgcaggggcccgagattgggtgtgcgcgcgacgaggaaaacttccgaacggtcccagccacgtgggaggcgccagcccatccctaaagatcggcgcaccactggcaagtcctggggaaggccaggatacccttggcccctgtatgggaatgagggcctcggctgggcagggtggctcctgtccccccgcggttctcgcccttcatggggccccaccgacccccggcataaatcgcgcaacttgggtaaggtcatcgataccctaacgtgcggttttgccgacctcatggggtacatacccgtcgttggcgctcccgttggcggcgttgccagagccctcgcccatggggtgagggttctggaggacgggataaattatgcaacggggaatttgcccggttgctctttctctatctttctcttggccctcttgtcttgcatctctgtgccagtttccgcc

SEQ ID NO: 8 - DNA, 573 bp, Hepatitis C virus type (V)/3a
atgagcacacttcctaaacctcaaagaaaaaccaaaagaaacaccatccgtcgcccacaggacgtcaagttcccgggtggcggacagatcgttggtggagtatacgtgttgccgcgcaggggcccacgattgggtgtgcgcgcgacgcgtaaaacttctgaacggtcacagcctcgcggacgacgacagcctatccccaaggcgcgtcggagcgaaggccggtcctgggctcagcccgggtacccttggcccctctatggtaacgagggctgcgggtgggcagggtggctcctgtccccacgcggctcccgtccatcttggggcccaaacgacccccggcggaggtcccgcaatttgggtaaagtcatcgatacccttacgtgcggattcgccgacctcatggggtacatcccgctcgtcggcgctcccgtaggaggcgtcgcaagagccctcgcgcatggcgtgagggcccttgaagacgggataaatttcgcaacagggaacttgcccggttgctccttttctatcttccttcttgctctgttctcttgcttaattcatccagcagctagt

SEQ ID NO: 9 - DNA, 573 bp, Hepatitis C virus type 4a
atgagcacgaatcctaaacctcaaagaaaaaccaaacgtaacaccaaccgccgccccatggacgtaaagttcccgggtggtggccagatcgttggcggagtttacttgttgccgcgcaggggccccaggttgggtgtgcgcgcgactcgaaagacttcggagcggtcgcaacctcgtggcaggcgtcaacctatccccaaggcgcgccagccagagggcagatcctgggcgcagcccgggtacccttggcccctctatggcaatgagggctgcgggtgggcagggtggctcctgtctcctcgcggctctcggccatcttggggcccaaatgatccccggcggagatcgcgcaatctgggtaaggtcatcgataccctgacgtgcggcttcgccgacctcatgggatacatcccgatcgtgggcgcccccgtggggggcgtcgccagggctctggcgcatggcgtcagggctgtggaggacgggattaactatgcaacagggaatcttcccggttgctctttctctatcttccttttggcacttctttcgtgcctcactgttccagcgtcggct

SEQ ID NO: 10 - DNA, 573 bp, Hepatitis C virus type 4b
atgagcacaaatcctaaacctcaaagaaaaaccaaacgtaacaccaaccgtcgccccatggatgtgaaattcccgggcggcggccagatcgttggcggagtttacttgctgccgcgcaggggcccccggttgggtgtgcgcgcagctcggaagacttcggagcggtcacaacctcgtggcaggcgtcagcctatccccaaggcgcgccggtccgagggcaggtcctgggctcagcccgggtacccttggcccctttacggcaatgagggctgtgggtgggcagggtggctcctgtccccccgcggttccaggccgtcttggggccccaatgatccccggcgtaggtcccgtaatctgggtaaagtcatcgataccctgacgtgtggcttcgccgacctcatgggatacattccgctcgtaggcgcccctgtgggtggcgtcgccagggccctggcgcatggcgtcagggccgtggaggacggaattaactacgcaacagggaaccttcctggttgctctttctctatctttcttcttgcacttctctcgtgcctgacaacaccagcatctgcc

SEQ ID NO: 11 - DNA, 573 bp, Hepatitis C virus type 4c
atgagcacgaatcctaaacctcaaagaaaaaccaaacgtaacaccaaccgccgccccatggacgttaagttcccgggtggtggccagatcgttggcggagtttacttgttgccgcgcaggggccccaggttgggtgtgcgcgcgactaggaagacttcggagcggtcgcaacctcgtgggagacgccagcctatccccaaggcacgtcgatctgagggaaggtcctgggctcagcccgggtatccatggcctctttacggtaatgagggttgcgggtgggcgggatggctcctgtcaccccgtggctctcgaccgtcttggggtccaaatgatccccggcgaaggtcccgcaacttgggtaaggtcatcgatactctaacttgcggtttcgccgatctcatgggatacatcccgctcgtaggcgcccccgtgggcggcgtcgccagggccctggcacatggtgttagggctgtggaggacgggatcaattatgcaacagggaatcttcccggttgctctttctctatcttcctcttggcacttctttcgtgcctaactgttcccacctcggcc

SEQ ID NO: 12 - DNA, 573 bp, Hepatitis C virus type 4d
atgagcacgaatcctaaacctcaaagaaaaaccaaacgtaacaccaaccgccgcccaatggacgttaagttcccgggtggcggccagatcgttggcggagtttacttgttgccgcgcaggggccctagattgggtgtgcgcgcgactaggaagacttcggagcggtcgcaacctcgtgggaggcgccagcctatccccaaggcgcgccaactcgagggtaggtcctgggctcagcctgggtatccttggcccctttacggcaatgagggctgcgggtgggcgggatggctcctgtcaccccgtggctctcggccgtcttggggcccgaatgatccccggcggaggtcccgcaacttgggtaaggtcatcgataccctaacttgcggcttcgccgacctcatgggatacatcccggtcgtaggcgcccccgtgggtggcgtcgccagagccctggcgcatggcgtcaggcttctggaggacggggtcaattatgcaacagggaatcttcccggttgctctttctctatcttcctcttggcactgctctcgtgcctgactgttcccgcttcggcc

SEQ ID NO: 13 - DNA, 573 bp, Hepatitis C virus type 4e
atgagcacgaatcctaaacctcaaagaaaaaccaaacgtaacaccaaccgccgccccatggatgtaaaattcccgggtggtggtcagatcgttggcggagtttacttgttgccgcgcaggggccccaggttgggtgtgcgcgcgactcggaagacttcggagcggtcgcaacctcgcggcaggcgtcagcctatcccccaggcacgtcggtccgagggcaggtcctgggctcagcccgggtacccttggcctctttatggcaatgagggctgtgggtgggcagggtggctcctgtccccccgcggatctcggccatcttggggccaaaatgatccccggcgtaggtcccgcaatctgggtaaggtcatcgataccctgacgtgtggcttcgccgacctcatgggatacattccgctcgtcggcgccccagtaggtggcgtcgccagggccttggcgcatggcgtcagggccctggaggacggaatcaactatgcaacagggaatcttcctggttgctccttttctatcttcctacttgcacttttctcgtgcttgacaacaccggcatccgct

SEQ ID NO: 14 - DNA, 573 bp, Hepatitis C virus type 4f
atgagcacgaatcctaaacctcaaagaaaaaccaaacgtaacaccaaccgccgccctatggatgtaaaattcccaggcggcggccagatcgttggcggagtttacttgttgccgcgcaggggccccaggttgggtgtgcgcgcgactcggaagacttcggagcggtcgcaacctcgtggcaggcgtcagcctatccccaaggcacgtcggtccgagggtaggtcctgggctcagcccgggtacccatggcctctttacggtaatgaaggctgtgggtgggcaggttggctcctgtccccccgcggctctcgaccgtcttggggcccaaatgatccccggcggaggtcgcgcaatttgggtaaggtcatcgataccctcacgtgcggcttcgccgacctcatgggatacatcccgctcgtgggcgccccagtaggaggcgtcgccagagccctggcgcatggcgtcagggctgtggaggacgggatcaactatgcaacagggaaccttcctggttgctctttctctatcttcctcttggcacttctctcgtgcctaaccgtcccagcgtctgct

SEQ ID NO: 15 - DNA, 573 bp, Hepatitis C virus type 5a
atgagcacgaatcctaaacctcaaagaaaaaccaaaagaaacaccaaccgccgcccacaggacgtcaagttcccgggcggtggtcagatcgttggtggagtttacttgttgccgcgcaggggccctaggttgggtgtgcgcgcgactcggaagacttcagaacggtcgcaaccccgtgggcggcgccagcctattcccaaggcgcgccaacccacgggccggtcctggggtcaacccgggtacccttggcccctttacgccaatgagggcctcgggtgggcagggtggttgctctccccccgaggctctcggcctaattggggccccaatgacccccggcgaaaatcgcgcaatttgggtaaggtcatcgataccctaacgtgcggattcgccgacctcatggggtacatcccgctcgtaggcggccccgttgggggcgtcgcaagggctctcgcacacggtgtgagggttcttgaggacggggtaaactatgcaacagggaatttgcccggttgctctttctctatctttatccttgcacttctctcgtgcctgaccgtcccggcctctgca

SEQ ID NO: 16 - DNA, 573 bp, Hepatitis C virus type 6a
atgagcacacttccaaaaccccaaagaaaaaccaaaagaaacaccaaccgtcgcccaacggacgtcaagttcccgggtggcggtcagatcgttggcggagtttacttgttgccgcgcaggggcccccggttgggtgtgcgcgcgacgagaaagacttccgagcgatcccagcccagaggcaggcgccaacctataccaaaggcgcgccagccccagggcaggcactgggctcagcccggatacccttggcctctttatggaaacgagggctgtgggtgggcaggttggctcctgtccccccgcggctcccggccacattggggccccaatgacccccggcgtcgatcccggaatttgggtaaggtcatcgataccctaacgtgtgggttcgccgatctcatggggtacattcccgtcgtgggcgcgcctttgggcggcgtcgcggctgcgctcgcacatggcgtgagggcaatcgaggacgggatcaattatgcaacagggaatctccccggttgctctttctctatcttccttttggcactactctcgtgcctcacaacgccagcttcggct

SEQ ID NO: 17 - DNA, 573 bp, Hepatitis C virus
  Hepatitis C virus C gene consensus sequence
atgagcacgaatcctaaacctcaaagaaaaaccaaacgtaacaccaaccgccgcccacaggacgtcaagttcccgggcggtggtcagatcgttggtggagtttacttgttgccgcgcaggggccccaggttgggtgtgcgcgcgactaggaagacttccgagcggtcgcaacctcgtggaaggcgacagcctatccccaaggctcgccggcccgagggcaggtcctgggctcagcccgggtacccttggcccctctatggcaatgagggcttcgggtgggcaggatggctcctgtccccccgcggctctcggcctagttggggccccactgacccccggcgtaggtcgcgcaatttgggtaaggtcatcgataccctcacgtgcggcttcgccgacctcatggggtacatcccgctcgtcggcgcccccgtagggggcgtcgccagggccctggcgcatggcgtcagggttctggaggacggggtgaactatgcaacagggaatttgcccggttgctctttctctatcttcctcctggctctgctgtcctgcctgaccgtcccagcttctgct

SEQ ID NO: 18 - protein, 191 aa, Hepatitis C virus type I/1a
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASA

SEQ ID NO: 19 - protein, 191 aa, Hepatitis C virus type II/ib
MSTNPKPQRKTKRNTNRRPQDVKFPGGGQIVGGVYLLPRRGPRLGVRATRKTSERSQPRGRRQPIPKARRPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASA

SEQ ID NO: 20 - protein, 191 aa, Hepatitis C virus type III/2a
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNYATGNLPGCSFSIFLLALLSCITIPVSA

SEQ ID NO: 21 - protein, 191 aa, Hepatitis C virus type IV/2b
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGKVIDTITCGFADLMGYIPVVGAPVGGVARALAHGVRVLEDGINYATGNLPGCSFSIFLLALLSCFTVPVSA

SEQ ID NO: 22 - protein, 191 aa, Hepatitis C virus type 2c
MSTNPKPQRKTKRNTNRRPQDVKFPGGGQIVGGVYLLPRRGPRLGVRATRKTSERSQPRGRRQPIPKDRRTTGKSWGRPGYPWPLYGNEGLGWAGWLLSPRGSRPSWGPTDPRHKSRNLGKVIDTLTCGFADLMGYIPVVGAPVGGVARALAHGVRVLEDGINYATGNLPGCSFSIFLLALLSCISVPVSA

SEQ ID NO: 23 - protein, 191 aa, Hepatitis C virus type (V)/3a
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAAS

SEQ ID NO: 24 - protein, 191 aa, Hepatitis C virus type 4a
MSTNPKPQRKTKRNTNRRPMDVKFPGGGQIVGGVYLLPRRGPRLGVRATRKTSERSQPRGRRQPIPKARQPEGRSWAQPGYPWPLYGNEGCGWAGWLLSPRGSRPSWGPNDPRRRSRNLGKVIDTLTCGFADLMGYIPIVGAPVGGVARALAHGVRAVEDGINYATGNLPGCSFSIFLLALLSCLTVPASA

SEQ ID NO: 25 - protein, 191 aa, Hepatitis C virus type 4b
MSTNPKPQRKTKRNTNRRPMDVKFPGGGQIVGGVYLLPRRGPRLGVRAARKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRAVEDGINYATGNLPGCSFSIFLLALLSCLTTPASA

SEQ ID NO: 26 - protein, 191 aa, Hepatitis C virus type 4c
MSTNPKPQRKTKRNTNRRPMDVKFPGGGQIVGGVYLLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRAVEDGINYATGNLPGCSFSIFLLALLSCLTVPTSA

SEQ ID NO: 27 - protein, 191 aa, Hepatitis C virus type 4d
MSTNPKPQRKTKRNTNRRPMDVKFPGGGQIVGGVYLLPRRGPRLGVRATRKTSERSQPRGRRQPIPKARQLEGRSWAQPGYPWPLYGNEGCGWAGWLLSPRGSRPSWGPNDPRRRSRNLGKVIDTLTCGFADLMGYIPVVGAPVGGVARALAHGVRLLEDGVNYATGNLPGCSFSIFLLALLSCLTVPASA

SEQ ID NO: 28 - protein, 191 aa, Hepatitis C virus type 4e
MSTNPKPQRKTKRNTNRRPMDVKFPGGGQIVGGVYLLPRRGPRLGVRATRKTSERSQPRGRRQPIPQARRSEGRSWAQPGYPWPLYGNEGCGWAGWLLSPRGSRPSWGQNDPRRRSRNLGKVIDTLTCGFADLMGYIPLVGAPVGGVARALAHGVRALEDGINYATGNLPGCSFSIFLLALFSCLTTPASA

SEQ ID NO: 29 - protein, 191 aa, Hepatitis C virus type 4f
MSTNPKPQRKTKRNTNRRPMDVKFPGGGQIVGGVYLLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRAVEDGINYATGNLPGCSFSIFLLALLSCLTVPASA

SEQ ID NO: 30 - protein, 191 aa, Hepatitis C virus type 5a
MSTNPKPQRKTKRNTNRRPQDVKFPGGGQIVGGVYLLPRRGPRLGVRATRKTSERSQPRGRRQPIPKARQPTGRSWGQPGYPWPLYANEGLGWAGWLLSPRGSRPNWGPNDPRRKSRNLGKVIDTLTCGFADLMGYIPLVGGPVGGVARALAHGVRVLEDGVNYATGNLPGCSFSIFILALLSCLTVPASA

SEQ ID NO: 31 - protein, 191 aa, Hepatitis C virus type 6a
MSTLPKPQRKTKRNTNRRPTDVKFPGGGQIVGGVYLLPRRGPRLGVRATRKTSERSQPRGRRQPIPKARQPQGRHWAQPGYPWPLYGNEGCGWAGWLLSPRGSRPHWGPNDPRRRSRNLGKVIDTLTCGFADLMGYIPVVGAPLGGVAAALAHGVRAIEDGINYATGNLPGCSFSIFLLALLSCLTTPASA

SEQ ID NO: 32 - protein, 132 aa, Hepatitis C virus
  Hepatitis C virus C gene consensus sequence
MSTPKPQRTRNTRPDKFPGGGQIVGGVYLPRRGPRGVRRKSERSQPRGRRQPIPRGWPGPWPYEGWAGWLLSPGSPWGDPRSRNGVIDTTCADLMGYPVGPGGAALAHGVREDGNATGNPGCFSIFLALSCP

SEQ ID NO: 33 - RNA, 33 nt, Artificial Sequence
  Trans-cleaving hammerhead ribozyme
nnnnnncugangarncnnnnnngnygaaacnnn

SEQ ID NO: 34 - DNA, 9587 bp, Hepatitis C virus type 1b
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgttaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccggcccgagggtaggacctgggctcagcccgggtacccttggcccctctatggcaacgagggtatggggtgggcaggatggctcctgtcaccccgtggctctcggcctagttggggccccacagacccccggcgtaggtcgcgtaatttgggtaaggtcatcgatacccttacatgcggcttcgccgacctcatggggtacattccgcttgtcggcgcccccctaggaggcgctgccagggccctggcgcatggcgtccgggttctggaggacggcgtgaactatgcaacagggaatctgcccggttgctctttctctatcttcctcttagctttgctgtcttgtttgaccatcccagcttccgcttacgaggtgcgcaacgtgtccgggatataccatgtcacgaacgactgctccaactcaagtattgtgtatgaggcagcggacatgatcatgcacacccccgggtgcgtgccctgcgtccgggagagtaatttctcccgttgctgggtagcgctcactcccacgctcgcggccaggaacagcagcatccccaccacgacaatacgacgccacgtcgatttgctcgttggggcggctgctctctgttccgctatgtacgttggggatctctgcggatccgtttttctcgtctcccagctgttcaccttctcacctcgccggtatgagacggtacaagattgcaattgctcaatctatcccggccacgtatcaggtcaccgcatggcttgggatatgatgatgaactggtcacctacaacggccctagtggtatcgcagctactccggatcccacaagccgtcgtggacatggtggcgggggcccactggggtgtcctagcgggccttgcctactattccatggtggggaactgggctaaggtcttgattgtgatgctactctttgctggcgttgacgggcacacccacgtgacagggggaagggtagcctccagcacccagagcctcgtgtcctggctctcacaagggccatctcagaaaatccaactcgtgaacaccaacggcagctggcacatcaacaggaccgctctgaattgcaatgactccctccaaactgggttcattgctgcgctgttctacgcacacaggttcaacgcgtccggatgtccagagcgcatggccagctgccgccccatcgacaagttcgctcaggggtggggtcccatcactcacgttgtgcctaacatctcggaccagaggccttattgctggcactatgcaccccaaccgtgcggtattgtacccgcgtcgcaggtgtgtggcccagtgtattgcttcaccccgagtcctgttgtggtggggacgaccgaccgttccggagtccccacgtatagctggggggagaatgagacagacgtgctgctactcaacaacacgcggccgccgcaaggcaactggttcggctgtacatggatgaatagcaccgggttcaccaagacgtgcgggggccccccgtgtaacatcgggggggttggcaacaacaccttgatttgccccacggattgcttccgaaagcaccccgaggccacttacaccaaatgcggctcgggtccttggttgacacctaggtgtctagttgactacccatacagactttggcactacccctgcactatcaattttaccatcttcaaggtcaggatgtacgtggggggcgtggagcacaggctcaacgccgcgtgcaattggacccgaggagagcgctgtgacctggaggacagggatagatcagagcttagcccgctgctattgtctacaacggagtggcaggtactgccctgttcctttaccaccctaccggctctgtccactggattgatccacctccatcagaatatcgtggacgtgcaatacctgtacggtgtagggtcagtggttgtctccgtcgtaatcaaatgggagtatgttctgctgctcttccttctcctggcggacgcgcgcgtctgtgcctgcttgtggatgatgctgctgatagcccaggctgaggccaccttagagaacctggtggtcctcaatgcggcgtctgtggccggagcgcatggccttctctccttcctcgtgttcttctgcgccgcctggtacatcaaaggcaggctggtccctggggcggcatatgctctctatggcgtatggccgttgctcctgctcttgctggctttaccaccacgagcttatgccatggaccgagagatggctgcatcgtgcggaggcgcggtttttgtaggtctggtactcttgaccttgtcaccatactataaggtgttcctcgctaggctcatatggtggttacaatattttatcaccagggccgaggcgcacttgcaagtgtgggtcccccctcttaatgttcggggaggccgcgatgccatcatcctccttacatgcgcggtccatccagagctaatctttgacatcaccaaactcctgctcgccatactcggtccgctcatggtgctccaagctggcataaccagagtgccgtacttcgtgcgcgctcaagggctcattcatgcatgcatgttagtgcggaaggtcgctgggggtcattatgtccaaatggccttcatgaagctgggcgcgctgacaggcacgtacatttacaaccatcttaccccgctacgggattgggcccacgcgggcctacgagaccttgcggtggcagtggagcccgtcgtcttctccgacatggagaccaagatcatcacctggggagcagacaccgcggcgtgtggggacatcatcttgggtctgcccgtctccgcccgaaggggaaaggagatactcctgggcccggccgatagtcttgaagggcgggggtggcgactcctcgcgcccatcacggcctactcccaacagacgcggggcctacttggttgcatcatcactagccttacaggccgggacaagaaccaggtcgagggagaggttcaggtggtttccaccgcaacacaatccttcctggcgacctgcgtcaacggcgtgtgttggaccgtttaccatggtgctggctcaaagaccttagccggcccaaaggggccaatcacccagatgtacactaatgtggaccaggacctcgtcggctggcaggcgccccccggggcgcgttccttgacaccatgcacctgtggcagctcagacctttacttggtcacgagacatgctgacgtcattccggtgcgccggcggggcgacagtagggggagcctgctctcccccaggcctgtctcctacttgaagggctcttcgggtggtccactgctctgcccttcggggcacgctgtgggcatcttccgggctgccgtatgcacccggggggttgcgaaggcggtggactttgtgcccgtagagtccatggaaactactatgcggtctccggtcttcacggacaactcatcccccccggccgtaccgcagtcatttcaagtggcccacctacacgctcccactggcagcggcaagagtactaaagtgccggctgcatatgcagcccaagggtacaaggtgctcgtcctcaatccgtccgttgccgctaccttagggtttggggcgtatatgtctaaggcacacggtattgaccccaacatcagaactggggtaaggaccattaccacaggcgcccccgtcacatactctacctatggcaagtttcttgccgatggtggttgctctgggggcgcttatgacatcataatatgtgatgagtgccattcaactgactcgactacaatcttgggcatcggcacagtcctggaccaagcggagacggctggagcgcggcttgtcgtgctcgccaccgctacgcctccgggatcggtcaccgtgccacacccaaacatcgaggaggtggccctgtctaatactggagagatccccttctatggcaaagccatccccattgaagccatcagggggggaaggcatctcattttctgtcattccaagaagaagtgcgacgagctcgccgcaaagctgtcaggcctcggaatcaacgctgtggcgtattaccgggggctcgatgtgtccgtcataccaactatcggagacgtcgttgtcgtggcaacagacgctctgatgacgggctatacgggcgactttgactcagtgatcgactgtaacacatgtgtcacccagacagtcgacttcagcttggatcccaccttcaccattgagacgacgaccgtgcctcaagacgcagtgtcgcgctcgcagcggcggggtaggactggcaggggtaggagaggcatctacaggtttgtgactccgggagaacggccctcgggcatgttcgattcctcggtcctgtgtgagtgctatgacgcgggctgtgcttggtacgagctcacccccgccgagacctcggttaggttgcgggcctacctgaacacaccagggttgcccgtttgccaggaccacctggagttctgggagagtgtcttcacaggcctcacccacatagatgcacacttcttgtcccagaccaagcaggcaggagacaacttcccctacctggtagcataccaagccacggtgtgcgccagggctcaggccccacctccatcatgggatcaaatgtggaagtgtctcatacggctgaaacctacgctgcacgggccaacacccttgctgtacaggctgggagccgtccaaaatgaggtcaccctcacccaccccataaccaaatacatcatggcatgcatgtcggctgacctggaggtcgtcactagcacctgggtgctggtgggcggagtccttgcagctctggccgcgtattgcctgacaacaggcagtgtggtcattgtgggtaggattatcttgtccgggaggccggctattgttcccgacagggagcttctctaccaggagttcgatgaaatggaagagtgcgccacgcacctcccttacattgagcagggaatgcagctcgccgagcagttcaagcagaaagcgctcgggttactgcaaacagccaccaaacaagcggaggctgctgctcccgtggtggagtccaagtggcgagcccttgagacattctgggcgaagcacatgtggaatttcatcagcgggatacagtacttagcaggcttatccactctgcctgggaaccccgcaatagcatcattgatggcattcacagcctctatcaccagcccgctcaccacccaaagtaccctcctgtttaacatcttgggggggtgggtggctgcccaactcgccccccccagcgccgcttcggctttcgtgggcgccggcatcgccggtgcggctgttggcagcataggccttgggaaggtgcttgtggacattctggcgggttatggagcaggagtggccggcgcgctcgtggcctttaaggtcatgagcggcgagatgccctctaccgaggacctggtcaatctacttcctgccatcctctctcctggcgccctggtcgtcggggtcgtgtgtgcagcaatactgcgtcggcacgtgggtccgggagagggggctgtgcagtggatgaaccggctgatagcgttcgcctcgcggggtaatcacgtttcccccacgcactatgtgcctgagagcgacgccgcagcgcgtgttactcagatcctctccagccttaccatcactcagctgctgaaaaggctccaccagtggattaatgaggactgctccacaccgtgttccggctcgtggctaagggatgtttgggactggatatgcacggtgttgactgacttcaagacctggctccagtccaagctcctgccgcagctaccgggagtcccttttttctcgtgccaacgcgggtacaagggagtctggcggggagacggcatcatgcaaaccacctgcccatgtggagcacagatcaccggacatgtcaaaaacggttccatgaggatcgtcgggcctaagacctgcagcaacacgtggcatggaacattccccatcaacgcatacaccacgggcccctgcacaccctctccagcgccaaactattctagggcgctgtggcgggtggccgctgaggagtacgtggaggtcacgcgggtgggggatttccactacgtgacgggcatgaccactgacaacgtaaagtgcccatgccaggttccggctcctgaattcttctcggaggtggacggagtgcggttgcacaggtacgctccggcgtgcaggcctctcctacgggaggaggttacattccaggtcgggctcaaccaatacctggttgggtcacagctaccatgcgagcccgaaccggatgtagcagtgctcacttccatgctcaccgacccctcccacatcacagcagaaacggctaagcgtaggttggccagggggtctcccccctccttggccagctcttcagctagccagttgtctgcgccttccttgaaggcgacatgcactacccaccatgtctctccggacgctgacctcatcgaggccaacctcctgtggcggcaggagatgggcgggaacatcacccgcgtggagtcggagaacaaggtggtagtcctggactctttcgacccgcttcgagcggaggaggatgagagggaagtatccgttccggcggagatcctgcggaaatccaagaagttccccgcagcgatgcccatctgggcgcgcccggattacaaccctccactgttagagtcctggaaggacccggactacgtccctccggtggtgcacgggtgcccgttgccacctatcaaggcccctccaataccacctccacggagaaagaggacggttgtcctaacagagtcctccgtgtcttctgccttagcggagctcgctactaagaccttcggcagctccgaatcatcggccgtcgacagcggcacggcgaccgcccttcctgaccaggcctccgacgacggtgacaaaggatccgacgttgagtcgtactcctccatgcccccccttgagggggaaccgggggaccccgatctcagtgacgggtcttggtctaccgtgagcgaggaagctagtgaggatgtcgtctgctgctcaatgtcctacacatggacaggcgccttgatcacgccatgcgctgcggaggaaagcaagctgcccatcaacgcgttgagcaactctttgctgcgccaccataacatggtttatgccacaacatctcgcagcgcaggcctgcggcagaagaaggtcacctttgacagactgcaagtcctggacgaccactaccgggacgtgctcaaggagatgaaggcgaaggcgtccacagttaaggctaaactcctatccgtagaggaagcctgcaagctgacgcccccacattcggccaaatccaagtttggctatggggcaaaggacgtccggaacctatccagcaaggccgttaaccacatccactccgtgtggaaggacttgctggaagacactgtgacaccaattgacaccaccatcatggcaaaaaatgaggttttctgtgtccaaccagagaaaggaggccgtaagccagcccgccttatcgtattcccagatctgggagtccgtgtatgcgagaagatggccctctatgatgtggtctccacccttcctcaggtcgtgatgggctcctcatacggattccagtactctcctgggcagcgagtcgagttcctggtgaatacctggaaatcaaagaaaaaccccatgggcttttcatatgacactcgctgtttcgactcaacggtcaccgagaacgacatccgtgttgaggagtcaatttaccaatgttgtgacttggcccccgaagccagacaggccataaaatcgctcacagagcggctttatatcgggggtcctctgactaattcaaaagggcagaactgcggttatcgccggtgccgcgcgagcggcgtgctgacgactagctgcggtaacaccctcacatgttacttgaaggcctctgcagcctgtcgagctgcgaagctccaggactgcacgatgctcgtgaacggagacgaccttgtcgttatctgtgaaagcgcgggaacccaagaggacgcggcgagcctacgagtcttcacggaggctatgactaggtactctgccccccccggggacccgccccaaccagaatacgacttggagctgataacatcatgttcctccaatgtgtcggtcgcccacgatgcatcaggcaaaagggtgtactacctcacccgtgatcccaccacccccctcgcacgggctgcgtgggaaacagctagacacactccagttaactcctggctaggcaacattatcatgtatgcgcccactttgtgggcaaggatgattctgatgactcacttcttctccatccttctagcacaggagcaacttgaaaaagccctggactgccagatctacggggcctgttactccattgagccacttgacctacctcagatcattgaacgactccatggccttagcgcattttcactccatagttactctccaggtgagatcaatagggtggcttcatgcctcaggaaacttggggtaccacccttgcgagtctggagacatcgggccaggagcgtccgcgctaggctactgtcccagggggggagggccgccacttgtggcaagtacctcttcaactgggcagtgaagaccaaactcaaactcactccaatcccggctgcgtcccagctggacttgtccggctggttcgttgctggttacagcgggggagacatatatcacagcctgtctcgtgcccgaccccgctggttcatgctgtgcctactcctactttctgtaggggtaggcatctacctgctccccaaccgatgaacggggagctaaacactccaggccaataggccatttcctgttttttttttttttttttttttttttttttttttcttttccttctttttccctttttctttcttccttctttaatggtggctccatcttagccctagtcacggctagctgtgaaaggtccgtgagccgcatgactgcagagagtgctgatactggcctctctgcagatcatgt

SEQ ID NO: 35 - protein, 3010 aa, Hepatitis C virus type 1b
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADMIMHTPGCVPCVRESNFSRCWVALTPTLAARNSSIPTTTIRRHVDLLVGAAALCSAMYVGDLCGSVFLVSQLFTFSPRRYETVQDCNCSIYPGHVSGHRMAWDMMMNWSPTTALVVSQLLRIPQAVVDMVAGAHWGVLAGLAYYSMVGNWAKVLIVMLLFAGVDGHTHVTGGRVASSTQSLVSWLSQGPSQKIQLVNTNGSWHINRTALNCNDSLQTGFIAALFYAHRFNASGCPERMASCRPIDKFAQGWGPITHVVPNISDQRPYCWHYAPQPCGIVPASQVCGPVYCFTPSPVVVGTTDRSGVPTYSWGENETDVLLLNNTRPPQGNWFGCTWMNSTGFTKTCGGPPCNIGGVGNNTLICPTDCFRKHPEATYTKCGSGPWLTPRCLVDYPYRLWHYPCTINFTIFKVRMYVGGVEHRLNAACNWTRGERCDLEDRDRSELSPLLLSTTEWQVLPCSFTTLPALSTGLIHLHQNIVDVQYLYGVGSVVVSVVIKWEYVLLLFLLLADARVCACLWMMLLIAQAEATLENLVVLNAASVAGAHGLLSFLVFFCAAWYIKGRLVPGAAYALYGVWPLLLLLLALPPRAYAMDREMAASCGGAVFVGLVLLTLSPYYKVFLARLIWWLQYFITRAEAHLQVWVPPLNVRGGRDAIILLTCAVHPELIFDITKLLLAILGPLMVLQAGITRVPYFVRAQGLIHACMLVRKVAGGHYVQMAFMKLGALTGTYIYNHLTPLRDWAHAGLRDLAVAVEPVVFSDMETKIITWGADTAACGDIILGLPVSARRGKEILLGPADSLEGRGWRLLAPITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQSFQVAHLHAPTGSGKSTKVPAAYAAQGYKVLVLNPSVAATLGFGAYMSKAHGIDPNIRTGVRTITTGAPVTYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSGLGINAVAYYRGLDVSVIPTIGDVVVVATDALMTGYTGDFDSVIDCNTCVTQTVDFSLDPTFTIETTTVPQDAVSRSQRRGRTGRGRRGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVTSTWVLVGGVLAALAAYCLTTGSVVIVGRIILSGRPAIVPDRELLYQEFDEMEECATHLPYIEQGMQLAEQFKQKALGLLQTATKQAEAAAPVVESKWRALETFWAKHMWNFISGIQYLAGLSTLPGNPAIASLMAFTASITSPLTTQSTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPQLPGVPFFSCQRGYKGVWRGDGIMQTTCPCGAQITGHVKNGSMRIVGPKTCSNTWHGTFPINAYTTGPCTPSPAPNYSRALWRVAAEEYVEVTRVGDFHYVTGMTTDNVKCPCQVPAPEFFSEVDGVRLHRYAPACRPLLREEVTFQVGLNQYLVGSQLPCEPEPDVAVLTSMLTDPSHITAETAKRRLARGSPPSLASSSASQLSAPSLKATCTTHHVSPDADLIEANLLWRQEMGGNITRVESENKVVVLDSFDPLRAEEDEREVSVPAEILRKSKKFPAAMPIWARPDYNPPLLESWKDPDYVPPVVHGCPLPPIKAPPIPPPRRKRTVVLTESSVSSALAELATKTFGSSESSAVDSGTATALPDQASDDGDKGSDVESYSSMPPLEGEPGDPDLSDGSWSTVSEEASEDVVCCSMSYTWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSKAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPRWFMLCLLLLSVGVGIYLLPNR

SEQ ID NO: 36 - DNA, 59 bp, Artificial Sequence
  Hammerhead ribozyme
ggctggcctgatgagtccgtgaggacgaaacggtacccggtaccgtcgccagcccccga

SEQ ID NO: 37 - DNA, 59 bp, Artificial Sequence
  Hammerhead ribozyme
tgcagatcatgtgacggatctagatccgtcctgatgagtccgtgaggacgaacatgatc

SEQ ID NO: 38 - DNA, 20 bp, Hepatitis C virus CG1B strain
gccagcccccgattgggggc

SEQ ID NO: 39 - RNA, 20 nt, Hepatitis C virus CG1B strain
gccagcccccgauugggggc

SEQ ID NO: 40 - RNA, 20 nt, Hepatitis C virus CG1B strain
  isolated
accagcccccgauugggggc

SEQ ID NO: 41 - RNA, 48 nt, Hepatitis C virus CG1B strain
ccgcaugacugcagagagugcugauacuggccucucugcagaucaugu

SEQ ID NO: 42 - RNA, 48 nt, Hepatitus C virus CG1B strain
ccgcuugacugcagagagugcugauacuggccucucugcagaucaagu

SEQ ID NO: 43 - DNA, 29 bp, Artificial Sequence
  Primer
cggtacccggtaccgtcgccagcccccga

SEQ ID NO: 44 - DNA, 29 bp, Artificial Sequence
  Primer
acgtctagtacactgcctagatctaggca

SEQ ID NO: 45 - DNA, 26 bp, Artificial Sequence
  Primer
tccgtgaggacgaaacggtacccggt

SEQ ID NO: 46 - DNA, 26 bp, Artificial Sequence
  Primer
agatctaggcaggactactcaggcac

SEQ ID NO: 47 - DNA, 25 bp, Artificial Sequence
  Primer
ggctggcctgatgagtccgtgagga

SEQ ID NO: 48 - DNA, 25 bp, Artificial Sequence
  Primer
actcaggcactcctgcttgtactag

SEQ ID NO: 49 - DNA, 9646 bp, Hepatitis C virus type 1a clone H77
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccttctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctacatcagctggtgcatgtggtggcttcagtattttctgaccagagtagaagcgcaactgcacgtgtgggttccccccctcaacgtccggggggggcgcgatgccgtcatcttactcatgtgtgttgtacacccgactctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaggcggtggactttatccctgtggagaacctagagacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccctttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctacagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccaagcagaggttatcacccctgctgtccagaccaactggcagaaactcgaggtcttctgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctaccgcctttgtgggcgctggcttagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacataggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggccagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacccgcagaaattctgcggaagtctcggagattcgcccgggccctgcccgtttgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaggccatttcctgtttttttttttttttttttttttttttttttttttttttttttttttttttctttttttttttttttttccttttttttttttttttttttctttccttcttttttcctttcttttccttccttctttaatggtggctccatcttagccctagtcacggctagctgtgaaaggtccgtgagccgcatgactgcagagagtgctgatactggcctctctgcagatcatgt

SEQ ID NO: 50 - DNA, 9711 bp, Hepatitis C virus type 2b clone pJ6CF
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcgcagaccaccgggcgcctcaccagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcactctctttactctcacccccgggtataagacccttctcagccggtttttgtggtggttgtgctatcttctgaccctgggggaagctatggtccaggagtgggcaccacctatgcaggtgcgcggtggccgtgatggcatcatatgggccgtcgccatattctacccaggtgtggtgtttgacataaccaagtggctcttggcggtgcttgggcctgcttacctcctaaaaggtgctttgacgcgcgtgccgtacttcgtcagggctcacgctctactgaggatgtgcaccatggcaaggcatctcgcggggggcaggtacgtccagatggcgctactagcccttggcaggtggactggcacttacatctatgaccacctcacccctatgtcggattgggctgctagtggcctgcgggacctggcggtcgccgttgagcctatcatcttcagtccgatggagaagaaagtcattgtctggggagcggagacagctgcttgtggggacattttacacggacttcccgtgtccgcccgacttggtcgggaggtcctccttggcccagctgatggctatacctccaaggggtggagtcttctcgcccccatcactgcttacgcccagcagacacgtggccttttgggcaccatagtggtgagcatgacggggcgcgacaagacagaacaggctggggaaattcaggtcctgtccacagtcactcagtccttcctcggaacatccatctcgggggttttgtggactgtctaccatggagctggcaacaagactctggccggctcacggggtccggtcacgcagatgtactccagtgctgagggggacttagtagggtggcccagcccccctgggactaaatctttggagccgtgcacgtgtggagcggtcgacctgtacctggtcacgcggaacgctgatgtcatcccggctcgaagacgcggggacaaacggggagcgctactctccccgagacctctttccaccttgaaggggtcctcaggaggcccggtgctatgccccaggggccacgctgtcggagtcttccgggcagctgtgtgctctcggggcgtggctaagtccatagatttcatccccgttgagacactcgacatcgtcacgcggtcccccacctttagtgacaacagcacaccacctgctgtgccccagacctatcaggtcgggtacttgcatgccccgactggcagtggaaagagcaccaaagttcctgtcgcatatgctgctcaggggtataaagtgctagtgcttaatccctcagtggctgccaccctggggtttggggcgtacttgtctaaggcacatggcatcaatcccaacattaggactggagtcaggactgtgacgaccggggcgcccatcacgtactccacatatggcaaattcctcgccgatgggggctgtgcgggcggcgcctacgacatcatcatatgtgatgaatgccatgccgtggactctaccaccatccttggcatcggaacagtccttgatcaagcagagacagctggggtcagactaactgtgctggctacagctacgccccctgggtcagtgacaaccccccaccccaacatagaggaggtggcccttgggcaggagggcgagatccccttctatgggagggcgattcccctgtcttacatcaagggaggaagacatctgatcttctgccattcaaagaaaaagtgtgacgagctcgcggcggcccttcggggtatgggcttgaactcagtggcatactacagagggttggacgtctccgtaataccaactcagggagacgtagtggtcgtcgccaccgacgccctcatgacagggtatactggggactttgactccgtgatcgactgcaacgtagcggtcactcaagttgtagacttcagtttagaccccacattcaccataaccacacagattgtccctcaagacgctgtctcacgtagccagcgccggggtcgcacgggtaggggaagactgggcatttataggtatgtttccactggtgagcgagcctcaggaatgtttgacagtgtagtgctctgtgagtgctacgacgcaggggccgcatggtatgagctcacaccatcggagaccaccgtcaggctcagggcgtatttcaacacgcccggtttgcctgtgtgccaagaccatcttgagttttgggaggcagttttcaccggcctcacacacatagatgcccacttcctttcccaaacaaagcaatcgggggaaaatttcgcatacttaacagcctaccaggctacagtgtgcgctagggccaaagccccccccccgtcctgggacgtcatgtggaagtgtttgactcgactcaagcccacactcgtgggccccacacctctcctgtaccgcttgggctctgttaccaacgaggtcaccctcacacatcccgtgacgaaatacatcgccacctgcatgcaagccgaccttgaggtcatgaccagcacatgggtcttggcagggggagtcttggcggccgtcgccgcgtattgcctggcgaccgggtgtgtttgcatcatcggccgcttgcacattaaccagcgagccgtcgttgcgccggacaaggaggtcctctatgaggcttttgatgagatggaggaatgtgcctctagggcggctctcattgaagaggggcagcggatagccgagatgctgaagtccaagatccaaggcttattgcagcaagcttccaaacaagctcaagacatacaacccactgtgcaggcttcatggcccaaggtagaacaattctgggccaaacacatgtggaacttcattagcggcatccaatacctcgcaggactatcaacactgccagggaaccctgcagtagcttccatgatggcgttcagtgccgccctcaccagtccgctgtcaacaagcaccactatccttctcaacattttggggggctggctagcatcccaaattgcaccacccgcgggggccactggcttcgttgtcagtggcctagtgggagctgccgtaggcagtataggcttaggtaaggtgctagtggacatcctggcagggtatggtgcgggcatttcgggggctctcgtcgcattcaagatcatgtctggcgagaagccctccatggaggatgtcgtcaacttgctgcctggaattctgtctccgggtgccttggtagtgggagtcatctgcgcggccattctgcgccgacacgtgggaccgggggaaggcgccgtccaatggatgaatagactcattgcctttgcttccagaggaaatcacgtcgcccccacccactacgtgacggagtcggatgcgtcgcagcgtgtgacccaactacttggctcccttaccataaccagcctgctcagaagactccacaactggattactgaggactgccccatcccatgcggcggctcgtggctccgcgatgtgtgggactgggtttgcaccatcctaacagactttaaaaattggctgacctccaaattattcccaaagatgcccggcctcccctttgtctcctgtcaaaaggggtacaagggcgtgtgggccggcactggcatcatgaccacacggtgtccttgcggcgccaatatctctggcaatgtccgcttgggctccatgagaatcacggggcctaagacctgcatgaatatctggcaggggacctttcctatcaattgttacacggagggccagtgcgtgccgaaacccgcgccaaactttaaggtcgccatctggagggtggcggcctcagagtacgcggaggtgacgcagcacgggtcataccactacataacaggactcaccactgataacttgaaagtcccctgccaactaccctctcccgagttcttttcctgggtggacggagtgcagatccataggtttgcccccacaccgaagccgtttttccgggatgaggtctcgttctgcgttgggcttaattcatttgtcgtcgggtcccagcttccttgcgaccctgaacccgacacagacgtattgatgtccatgctaacagatccatctcatatcacggcggagactgcagcgcggcgtttagcgcgggggtcacccccatccgaggcaagctcctcggcgagccagctatcggcaccatcgctgcgagccacctgcaccacccacggcaaagcctatgatgtggacatggtggatgctaacctgttcatggggggcgatgtgactcggatagagtctgggtccaaagtggtcgttctggactctctcgacccaatggtcgaagaaaggagcgaccttgagccttcgataccatcagaatacatgctccccaagaagaggttcccaccagctttaccggcctgggcacggcctgattacaacccaccgcttgtggaatcgtggaaaaggccagattaccaaccggccactgttgcgggctgtgctctccctcctcctaggaaaaccccgacgcctcccccaaggaggcgccggacagtgggcctaagtgaggactccataggagatgcccttcaacagctggccattaagtcctttggccagccccccccaagcggcgattcaggcctttccacgggggcgggcgctgccgattccggcagtcagacgcctcctgatgagttggccctttcggagacaggttccatctcttccatgccccccctcgagggggagcttggagatccagacctggagcctgagcaggtagagccccaaccccccccccaggggggggtggcagctcccggctcggactcggggtcctggtctacttgctccgaggaggacgactccgtcgtgtgctgctccatgtcatactcctggaccggggctctaataactccttgtagtcccgaagaggagaagttaccgattaaccccttgagcaactccctgttgcgatatcacaacaaggtgtactgtaccacaacaaagagcgcctcactaagggctaaaaaggtaacttttgataggatgcaagtgctcgactcctactacgactcagtcttaaaggacattaagctagcggcctccaaggtcaccgcaaggctcctcaccatggaggaggcttgccagttaaccccaccccattctgcaagatctaaatatgggtttggggctaaggaggtccgcagcttgtccgggagggccgttaaccacatcaagtccgtgtggaaggacctcctggaggactcagaaacaccaattcccacaaccattatggccaaaaatgaggtgttctgcgtggaccccaccaaggggggcaagaaagcagctcgccttatcgtttaccctgacctcggcgtcagggtctgcgagaagatggccctttatgacattacacaaaaacttcctcaggcggtgatgggggcttcttatggattccagtattcccccgctcagcgggtagagtttctcttgaaagcatgggcggaaaagaaggaccctatgggtttttcgtatgatacccgatgctttgactcaaccgtcactgagagagacatcaggactgaggagtccatatatcgggcctgctccttgcccgaggaggcccacactgccatacactcgctaactgagagactttacgtgggagggcctatgttcaacagcaagggccaaacctgcgggtacaggcgttgccgcgccagcggggtgctcaccactagcatggggaacaccatcacatgctacgtgaaagccttagcggcttgtaaagctgcagggataatcgcgcccacaatgctggtatgcggcgatgacttggttgtcatctcagaaagccaggggaccgaggaggacgagcggaacctgagagccttcacggaggctatgaccaggtattctgcccctcctggtgacccccccagaccggagtatgatctggagctgataacatcttgctcctcaaatgtgtctgtggcgctgggcccacaaggccgccgcagatactacctgaccagagaccctaccactccaatcgcccgggctgcctgggaaacagttagacactcccctgtcaattcatggctgggaaacatcatccagtacgccccgaccatatgggctcgcatggtcctgatgacacacttcttctccattctcatggctcaagacacgctggaccagaacctcaactttgagatgtacggagcggtgtactccgtgagtcccttggacctcccagctataattgaaaggttacatgggcttgacgctttttctctgcacacatacactccccacgaactgacacgggtggcttcagccctcagaaaacttggggcgccacccctcagagcgtggaagagccgggcacgtgcagtcagggcgtccctcatctcccgtggggggagagcggccgtttgcggtcgatatctcttcaattgggcggtgaagaccaagctcaaactcactccattgccggaagcgcgcctcctggatttatccagctggttcaccgtcggcgccggcgggggcgacatttatcacagcgtgtcgcgtgcccgaccccgcttattgctctttggcctactcctactttttgtaggggtaggccttttcctactccccgctcggtagagcggcacacattagctacactccatagctaactgtcccttttttttttttttttttttttttttttttttttttttttttttttttttttttttttttttttttttttttttttttttttttctttttttctcttttccttctttcttaccttattttactttctttcctggtggctccatcttagccctagtcacggctagctgtgaaaggtccgtgagccgcatgactgcagagagtgccgtaactggtctctctgcagatcatgt

SEQ ID NO: 51 - DNA, 9678 bp, Hepatitis C virus type 2a clone JFH-1
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcgcccagaagacgttaagttcccgggcggcggccagatcgttggcggagtatacttgttgccgcgcaggggccccaggttgggtgtgcgcacgacaaggaaaacttcggagcggtcccagccacgtgggagacgccagcccatccccaaagatcggcgctccactggcaaggcctggggaaaaccaggtcgcccctggcccctatatgggaatgagggactcggctgggcaggatggctcctgtccccccgaggctctcgcccctcctggggccccactgacccccggcataggtcgcgcaacgtgggtaaagtcatcgacaccctaacgtgtggctttgccgacctcatggggtacatccccgtcgtaggcgccccgcttagtggcgccgccagagctgtcgcgcacggcgtgagagtcctggaggacggggttaattatgcaacagggaacctacccggtttccccttttctatcttcttgctggccctgttgtcctgcatcaccgttccggtctctgctgcccaggtgaagaataccagtagcagctacatggtgaccaatgactgctccaatgacagcatcacttggcagctcgaggctgcggttctccacgtccccgggtgcgtcccgtgcgagagagtggggaatacgtcacggtgttgggtgccagtctcgccaaacatggctgtgcggcagcccggtgccctcacgcagggtctgcggacgcacatcgatatggttgtgatgtccgccaccttctgctctgctctctacgtgggggacctctgtggcggggtgatgctcgcggcccaggtgttcatcgtctcgccgcagtaccactggtttgtgcaagaatgcaattgctccatctaccctggcaccatcactggacaccgcatggcatgggacatgatgatgaactggtcgcccacggccaccatgatcctggcgtacgtgatgcgcgtccccgaggtcatcatagacatcgttagcggggctcactggggcgtcatgttcggcttggcctacttctctatgcagggagcgtgggcgaaggtcattgtcatccttctgctggccgctggggtggacgcgggcaccaccaccgttggaggcgctgttgcacgttccaccaacgtgattgccggcgtgttcagccatggccctcagcagaacattcagctcattaacaccaacggcagttggcacatcaaccgtactgccttgaattgcaatgactccttgaacaccggctttctcgcggccttgttctacaccaaccgctttaactcgtcagggtgtccagggcgcctgtccgcctgccgcaacatcgaggctttccggatagggtggggcaccctacagtacgaggataatgtcaccaatccagaggatatgaggccgtactgctggcactaccccccaaagccgtgtggcgtagtccccgcgaggtctgtgtgtggcccagtgtactgtttcacccccagcccggtagtagtgggcacgaccgacagacgtggagtgcccacctacacatggggagagaatgagacagatgtcttcctactgaacagcacccgaccgccgcagggctcatggttcggctgcacgtggatgaactccactggtttcaccaagacttgtggcgcgccaccttgccgcaccagagctgacttcaacgccagcacggacttgttgtgccctacggattgttttaggaagcatcctgatgccacttatattaagtgtggttctgggccctggctcacaccaaagtgcctggtccactacccttacagactctggcattacccctgcacagtcaattttaccatcttcaagataagaatgtatgtagggggggttgagcacaggctcacggccgcatgcaacttcactcgtggggatcgctgcgacttggaggacagggacaggagtcagctgtctcctctgttgcactctaccacggaatgggccatcctgccctgcacctactcagacttacccgctttgtcaactggtcttctccaccttcaccagaacatcgtggacgtacaatacatgtatggcctctcacctgctatcacaaaatacgtcgttcgatgggagtgggtggtactcttattcctgctcttagcggacgccagagtctgcgcctgcttgtggatgctcatcttgttgggccaggccgaagcagcattggagaagttggtcgtcttgcacgctgcgagtgcggctaactgccatggcctcctatattttgccatcttcttcgtggcagcttggcacatcaggggtcgggtggtccccttgaccacctattgcctcactggcctatggcccttctgcctactgctcatggcactgccccggcaggcttatgcctatgacgcacctgtgcacggacagataggcgtgggtttgttgatattgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

(19) United States
(12) Patent Application Publication
(10) Pub. No.: US 2009/0017070 A1
(43) Pub. Date: Jan. 15, 2009

(54) In vitro model for hepatitis c virion production

(76) Inventors: Jake T. Liang, N. Bethesda, MD (US); Theo Heller, Silver Springs, MD (US); Satoru Saito, Yokohama City (JP)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/664,375

(22) PCT Filed: Sep. 30, 2005
(86) PCT No.: PCT/US2005/035487

§ 371 Date: May 21, 2008

Related U.S. Application Data
(60) Provisional application No. 60/615,301, filed on Sep. 30, 2004.
(60) Provisional application No. 60/642,210, filed on Jan. 6, 2005.

(51) Int. Cl. A61K 39/29; C12N 15/63; C12Q 1/70; C12P 19/34; C12N 5/00

(52) U.S. Cl. 424/228.1; 435/320.1; 435/5; 435/325; 435/91.1

(57) ABSTRACT

The invention is related to a construct comprising a hepatitis C virus (HCV) nucleic acid sequence that comprises from 5′ to 3′ on the positive-sense nucleic acid a 5′ untranslated region (UTR), a full-length open reading frame (ORF) encoding an HCV polyprotein whose cleavage products form functional components of HCV virus particles and RNA replication machinery, and a 3′ untranslated region (UTR), said sequence being infectious, and, additionally, a ribozyme pair positioned to generate the 5′ and 3′ ends of said sequence when cleaved, and related methods of making and methods of using.

### RELATED APPLICATIONS

This application claims the benefit of U.S. Provisional Patent Application No. 60/615,301, filed Sep. 30, 2004; U.S. Provisional Patent Application No. 60/642,210, filed Jan. 6, 2005; and U.S. Provisional Patent Application filed Sep. 26, 2005, all of which are hereby expressly incorporated by reference in their entireties.

### BACKGROUND OF THE INVENTION

The hepatitis C virus (HCV) is an important cause of human illness worldwide (Liang, T. J. et al. 2000 Ann. Intern. Med. 132:296-305). Although it has proven to be a difficult public health problem, it has been no easier to study in the laboratory. A major impediment has been the lack of robust model systems to study the complete viral life cycle. HCV is a member of the Flaviviridae family of ≈9.6 kb, and it has a central ORF flanked by the 5′ and 3′ noncoding regions. The ORF is divided into the coding sequences for the structural proteins at the 5′ end and the nonstructural proteins at the 3′ end. Study of the biology of hepatitis C at a molecular level focused initially on expression and manipulation of individual viral proteins in tissue culture.

The development of the subgenomic and genomic replicons is a major breakthrough to understanding viral replication and viral-cell interactions and provides a means to test therapeutic targets (Lohmann, V. et al. 1999 Science 285:110-3; Ikeda, M. et al. 2002 J Virol 76:2997-3006). However, as yet, none of these systems produce viral particles, nor do they produce infectious virions. Although some infectious tissue culture systems have been described; in general, these systems have not been robust enough to study the complete viral life cycle (Shimizu, Y. K. et al. 1992 PNAS USA 89:5477-81; Sung, V. M. et al. 2003 J Virol 77:2134-46).

Why virion production has been such an elusive goal remains unclear; however, the promise of a system that produces authentic virions is clear. Not only would more of the biology of the virus become accessible for study, but also such a system would provide a means to screen a wider range of potential therapeutic compounds. There is evidence for an inverse relationship between viral replication in tissue culture and virulence in the host organism. This relationship is true for hepatitis A, and there is evidence that it may be true for HCV as well (Raychaudhuri, G. et al. 1998 J Virol 72:7467-75; Bukh, J. et al. 2002 PNAS USA 99:14416-21). Regardless of the reason for this difficulty, there is an urgent need to establish such a system if improved therapies are to be developed, particularly given the absence of a simple small-animal model of HCV infection. This need is especially true for genotype 1, given that this genotype is the major genotype of human infections worldwide and is the type most resistant to current therapies (Manns, M. P. et al. 2001 Lancet 358: 958-65; Fried, M. W. et al. 2002 N Engl. J Med 347:975-82).

### Segue to Invention

In this study, we describe an in vitro HCV replication system that is capable of producing viral particles in the culture medium. A full-length HCV construct, CG1b of genotype 1b, known to be infectious (Thomson, M. et al. 2001 Gastroenterology 121:1226-33), was placed between two ribozymes designed to generate the exact 5′ and 3′ ends of HCV when cleaved. By using this system, we showed that HCV proteins and positive and negative RNA strands were produced intracellularly, and viral particles that resemble authentic HCV virions were produced and secreted into the culture medium. This system provides a unique opportunity to further study the life cycle and biology of HCV and to test potential therapeutic targets.

### SUMMARY OF THE INVENTION

The invention is related to a construct comprising a hepatitis C virus (HCV) nucleic acid sequence that comprises from 5′ to 3′ on the positive-sense nucleic acid a 5′ untranslated region (UTR), a full-length open reading frame (ORF) encoding an HCV polyprotein whose cleavage products form functional components of HCV virus particles and RNA replication machinery, and a 3′ untranslated region (UTR), said sequence being infectious, and, additionally, a ribozyme pair positioned to generate the 5′ and 3′ ends of said sequence when cleaved, and related methods of making and methods of using.

### BRIEF DESCRIPTION OF THE DRAWINGS

FIG. 1. Structure and processing of the hepatitis C virus (HCV) polyprotein by cellular signal peptidases and virally encoded proteases (NS2/3 and NS3). The viral coding region is represented across the top. Boxes below indicate precursors and mature proteins generated by proteolytic processing events. Approximate sizes by sodium dodecyl sulfate-polyacrylamide electrophoresis of the mature proteins (p) and glycoproteins (gp) are indicated. See Table 3 for nucleotide and amino acid locations of the mature products.

FIG. 2. (A) Model showing the predicted RNA secondary and tertiary structure of the hepatitis C virus (HCV) 5′ untranslated region (H strain) and the adjacent core coding sequence showing the individual stem-loop structures important for internal ribosome entry site (IRES) function (SEQ ID NO: 1). (B) Computer-predicted secondary structure of the HCV 3′ UTR(H strain) and limited upstream sequence (SEQ ID NO: 2). Stem-loop (SL) structures and individual regions are labeled.

FIG. 3. Alignment of the consensus sequence of the C gene of the different genotypes of HCV. I/1a—SEQ ID NO: 3; II/1b—SEQ ID NO: 4; III/2a—SEQ ID NO: 5; IV/2b—SEQ ID NO: 6; 2c—SEQ ID NO: 7; (V)/3a—SEQ ID NO: 8; 4a—SEQ ID NO: 9; 4b—SEQ ID NO: 10; 4c—SEQ ID NO: 11; 4d—SEQ ID NO: 12; 4e—SEQ ID NO: 13; 4f —SEQ ID NO: 14; 5a—SEQ ID NO: 15; 6a—SEQ ID NO: 16. Consensus sequence of the C gene from all 52 HCV isolates studied is shown at the top (SEQ ID NO: 17). Invariant nucleotides within a consensus sequence are capitalized and variable nucleotides are shown in lowercase letters. However, nucleotides that were invariant among all 52 HCV isolates are shown as dashes in the alignment.

FIG. 4. Alignment of consensus sequence of deduced amino acid sequences of the C gene of the different genotypes of HCV. I/1a—SEQ ID NO: 18; II/1b—SEQ ID NO: 19; III/2a—SEQ ID NO: 20; IV/2b—SEQ ID NO: 21; 2c—SEQ ID NO: 22; (V)/3a—SEQ ID NO: 23; 4a—SEQ ID NO: 24; 4b—SEQ ID NO: 25; 4c—SEQ ID NO: 26; 4d—SEQ ID NO: 27; 4e—SEQ ID NO: 28; 4f-SEQ ID NO: 29; 5a—SEQ ID NO: 30; 6a—SEQ ID NO: 31. Consensus sequence of the C protein from all 52 HCV isolates studied is shown at the top (SEQ ID NO: 32).

FIG. 5. Trans-cleaving hammerhead ribozyme (SEQ ID NO: 33). Secondary structure model of the hammerhead ribozyme-substrate complex. Important nucleotides for catalytic activity and structural domains helices I to III are shown. Ribozyme nucleotides are in uppercase letters; substrate nucleotides are in lowercase letters. The arrow indicates the cleavage site. Nucleotides are numbered as described in Hertel et al. (1992 Nucleic Acid Res. 20:3252). represents any nucleotide; Y represents C or U; R represents A or G; his A, C or U.

FIG. 6. (A) Hepatitis C virus type 1b polyprotein mRNA (GenBank Accession number AF333324, SEQ ID NO: 34). (B) Hepatitis C virus type 1b polyprotein, amino acid sequence (GenBank Accession number AF333324, SEQ ID NO: 35).

FIG. 7. Construction of HCV-ribozyme plasmid. (A) The design of the construct is shown with the positions and sequences of the ribozymes (Rbz) flanking the 5′ (SEQ ID NO: 36) and 3′ ends (SEQ ID NO: 37) of the HCV CG1B sequence. The cleavage sites are indicated by arrows. The boxes shown 5′ and 3′ to the construct represent the promoter sequence (5′ end) and the simian virus 40 small T antigen intron and polyadenylation signal (3′ end). (B) An RNA gel with in vitro transcription products from pHr. The first lane shows molecular weight (MW) markers, and the second lane shows a sense transcript beginning at the 5′ end under the control of the T7 promoter. (Upper) The expected fragments at ≈9,500 and 5,400 nucleotides are indicated by arrows. The third lane shows an antisense transcript from the 3′ end under the control of a T3 promoter showing bands representing the full-length of the plasmid and a population of RNA ≈1,400 bp long that possibly represents a termination sequence or difficult secondary structure at that region. (Lower) The expected 150-nt fragment can be seen on this gel with longer exposure (both lanes labeled T7).

FIG. 8. Detection of HCV positive- and negative-strand RNAs. (Upper) The experiment. Shown are the total cellular RNA probed for the HCV core sequence, either positive or negative strand, and the findings when cellular RNA from pTRE-, pTHr-, or pTHrGND-transfected cells were probed for either positive- or negative-strand core sequence. (Lower) The control. Shown is the total cellular RNA probed for GAPDH messenger RNA. Note that the amounts are roughly comparable in the three lanes.

FIG. 9. Detection of HCV proteins by immunofluorescence. (A) Low-power view of cells transfected with pTHr and stained without primary antibody but with the secondary antibody. No fluorescence was seen. (B) Low-power view of cells transfected with pTHr and stained with anti-core. Multiple cells with fluorescence can be seen. (C) Low-power view of cells transfected with the control pTRE and stained with anti-core. There was no fluorescence. (D) High-power view of B. (E and F) High-power views of cells stained with anti-E2. Cells were transfected with pTRE (E) or pTHr (F). (G and H) High-power views of cells transfected with pTRE (G) and pTHr (H) and stained with anti-NS 5A.

FIG. 10. Detection of HCV proteins by Western blot. In each blot, the first lane shows cells transfected with pTHr and the second lane shows cells transfected with pTRE. The molecular weights are shown on the left of the blots. (Left) Blot was probed with anti-core. (Center) Blot probed with anti-E2. (Right) Blot probed with anti-NS5A.

FIG. 11. Sucrose density gradient analysis of culture medium of HCV-transfected cells. (A) (Lower) Results of the sucrose gradient for pTHr (solid lines) and pTHrGND (dotted lines) transfections. The buoyant density of the sucrose is plotted with the levels of HCV RNA measured by TaqMan PCR and HCV core protein measured by core ELISA. (Upper) Western blot for the E2 protein in the fractions of the sucrose gradient of the pTHr transfection. Each lane corresponds to the fraction number below it on the x axis of the graph. Three hundred microliters of each fraction was spun at 100,000×g for 90 min, and the pellet was resuspended in loading buffer and used for the Western blot. (B) Cryoelectron microscopy of fraction 5. (Bar, 100 nm.)

FIG. 12. Sequences of the 5′ and 3′ ends of HCV RNA. (A) The cDNA sequence for the 5′ end of the CG1B strain (a) (SEQ ID NO: 38), and the RACE results for the 5′ ends of in vitro transcribed RNA (b) (SEQ ID NO: 39) and of the HCV RNA from the culture medium (c) (SEQ ID NO: 40). (B) The cDNA sequences and the stem-loop structures of the 3′ ends of the CG1b strain (left) (SEQ ID NO: 41) and the HCV RNA from the medium (right) (SEQ ID NO: 42). Nucleotide changes are boxed.

FIG. 13. Production of infectious HCV in culture. (A). Full-length genomic cDNAs of HCV genotypes 1a (H77), 2b (J6), and 2a (JFH-1) were cloned into the HCV-ribozyme construct as described in FIG. 7. The constructs and the pTHr plasmid containing the CG1b HCV genome as described above were transfected into Huh7 cells and tested for virus production in the medium by using HCV core Ag ELISA. (B) HCV.JFH1-Rbz construct was transfected to Huh7 cells and treated with interferon one day after transfection. Viral production was monitored by measurement of HCV core Ag level in the medium. (C) Long-term culture of HCV.JFH1.Rbz transfected Huh7 cells were maintained with serial passage for the indicated duration. Culture medium was harvested at indicated times for HCV core Ag level and HCV RNA titer. (D) Millipore-filtered culture medium from HCV.JFH1-Rbz transfected cells were incubated with naïve Huh7 cells for 6 h followed by extensive washing with culture medium. Two days later the cells were subjected to immunofluorescence staining with antibodies to core antigen. Multiple foci of cells with positive intracellular staining were detected.

FIG. 14. In vivo infectivity of JFH-1 virus produced in tissue culture. Chimpanzee X0215 was first inoculated with 1 ml of the undiluted culture medium from mock-transfected Huh7 cells. Six weeks later, the chimpanzee was re-inoculated with 1 ml of the 10⁴ dilution (800 HCV genomes/ml) of culture medium from full-length JFH-1 RNA-transfected cells, and after 6 further weeks, inoculation was repeated with 1 ml of the 10³ dilution (8,000 genomes/ml). The course of infection is shown with arrows indicating the three inoculations. HCV RNA (copies/ml) and ALT (IU/L) levels are plotted; anti-HCV, HCV RNA, and liver biopsy results are given above the graph.

FIG. 15. Hepatitis C virus polyprotein gene, H77 clone (Genbank Accession No: AF009606, SEQ ID NO: 49).

FIG. 16. Hepatitis C virus clone pJ6CF, complete genome (GenBank Accession No.: AF177036, SEQ ID NO: 50)

FIG. 17. Hepatitis C virus polyprotein gene, clone JFH-1 (GenBank Accession No.: AB047639, SEQ ID NO:51).

### DETAILED DESCRIPTION OF THE PREFERRED EMBODIMENT

The hepatitis C virus (HCV) is a major cause of liver disease worldwide. The understanding of the viral life cycle has been hampered by the lack of a satisfactory cell culture system. The development of the HCV replicon system has been a major advance, but the system does not produce virions. In this study, we constructed an infectious HCV genotype 1b cDNA between two ribozymes that are designed to generate the exact 5′ and 3′ ends of HCV. A second construct with a mutation in the active site of the viral RNA-dependent RNA polymerase (RdRp) was generated as a control. The HCV-ribozyme expression construct was transfected into Huh7 cells. Both HCV structural (core, E1, E2) and nonstructural (NS5A) proteins were detected by immunofluorescence and Western blot. RNase protection assays showed positive- and negative-strand HCV RNA. Sequence analysis of the 5′ and 3′ ends provided further evidence of viral replication. Sucrose density gradient centrifugation of the culture medium revealed co-localization of HCV RNA and structural proteins in a fraction with a density of 1.16 g/ml, is the putative density of HCV virions. Electron microscopy showed viral particles of about 50 nm in diameter. The level of HCV RNA in the culture medium was as high as 10 million copies per ml. The HCV-ribozyme construct with the inactivating mutation in the RdRp did not show evidence of viral replication, assembly, and release. This system supports the production and secretion of high-level HCV virions and extends the repertoire of tools available for the study of HCV biology.

### DEFINITIONS

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3^{rd} ed., J. Wiley & Sons, Chichester, N.Y., 2001, and Fields Virology 4^{th} ed., Knipe D. M. and Howley P. M. eds, Lippincott Williams & Wilkins, Philadelphia 2001.

### Other RNA Viruses

In addition to HCV, the invention is applicable to other RNA viruses. The major animal virus families are listed in Table 1. The invention is applicable to members of the Retroviridae family (RNA reverse-transcribing viruses), Reoviridae family (dsRNA viruses), Arenaviridae, Bomaviridae, Bunyaviridae, Filoviridae, Orthomyxoviridae, Paramyxoviridae, and Rhabdoviridae families (Negative-sense ssRNA viruses), and Arteriviridae, Astroviridae, Caliciviridae, Coronaviridae, Flaviviridae, Picornaviridae, and Togaviridae families (Positive-sense ssRNA viruses).

### Flaviviridae

In addition to HCV, the invention is applicable to other members of the Flaviviridae family. Members of the Flaviviridae are listed in Table 2. The flaviviruses, pestiviruses, and hepaciviruses are members of the Flaviviridae family.

TABLE 1
Summary of Characteristics of Major Animal Virus Families

| | | | | | |
|---|---|---|---|---|---|
| | Nucleocapsid | | Virion | | |
| Family | morphology | Envelope | Morphology | Genome^{a} | Host^{b} |

dsDNA viruses

| | | | | | |
|---|---|---|---|---|---|
| Adenoviridae | Icosahedral | No | Icosahedral | 1 linear, 30-42 kb | V |
| Herpesviridae | Icosahedral | Yes | Spherical, | 1 linear, 120-220 kb | V |
| | | | tegument | | |
| Papillomaviridae | Icosahedral | No | Icosahedral | 1 circular, 8 kb | V |
| Polyomaviridae | Icosahedral | No | Icosahedral | 1 circular, 5 kb | V |
| Poxviridae | Ovoid | Yes | Ovoid | 1 linear, 130-375 kb | V, I |

ssDNA viruses

| | | | | | |
|---|---|---|---|---|---|
| Parvoviridae | Icosahedral | No | Icosahedral | 1 linear-sense, 5 kb | V, I |

RNA and DNA reverse-transcribing viruses

| | | | | | |
|---|---|---|---|---|---|
| Hepadnaviridae | Icosahedral | Yes | Spherical | 1 circular DNA, 3 kb | V |
| Retroviridae | Spherical or | Yes | Spherical | 1 linear RNA | V |
| | rod-shaped | | | dimer, 7-11 kb | |

dsRNA viruses

| | | | | | |
|---|---|---|---|---|---|
| Reoviridae | Icosahedral | No | Icosahedral, | 10-12 linear, 18-30 kb | V, I, P |
| | | | layered | | |

Negative-sense ssRNA viruses

| | | | | | |
|---|---|---|---|---|---|
| Arenaviridae | Helical filaments | Yes | Spherical | 2 linear, 5-7 kb | V |
| Bornaviridae | ND | Yes | Spherical | 1 linear, 9 kb | V |
| Bunyaviridae | Helical filaments | Yes | Spherical | 3 linear, 10-23 kb | V, I, P |
| Filoviridae | Helical filaments | Yes | Pleomorphic, | 1 linear, 19 kb | V |
| | | | filamentous | | |
| Orthomyxoviridae | Helical filaments | Yes | Pleomorphic, | 8 linear, 12-15 kb | V |
| | | | spherical | | |
| Paramyxoviridae | Helical filaments | Yes | Pleomorphic, | 1 linear, 15-16 kb | V |
| | | | spherical, | | |
| | | | filamentous | | |
| Rhabdoviridae | Coiled helical | Yes | Bullet-shaped | 1 linear, 11-15 kb | V, I, P |
| | filaments | | | | |

Positive-sense ssRNA viruses

| | | | | | |
|---|---|---|---|---|---|
| Arteriviridae | Icosahedral | Yes | Spherical | 1 linear, 13 kb | V |
| Astroviridae | Icosahedral | No | Icosahedral | 1 linear, 7-8 kb | V |
| Caliciviridae | Icosahedral | No | Icosahedral | 1 linear, 8 kb | V |
| Coronaviridae | Helical rod | Yes | Pleomorphic, | 1 linear, 20-33 kb | V |
| | | | spherical, | | |
| | | | rod-shaped | | |
| Flaviviridae | Polyhedral | Yes | Spherical | 1 linear, 10-12 kb | V, I |
| “Hepatitis E-like | Spherical | No | Spherical | 1 linear, 7 kb | V |
| viruses” | | | | | |
| Picornaviridae | Icosahedral | No | Icosahedral | 1 linear, 7-8 kb | V, I |
| Togaviridae | Icosahedral | Yes | Spherical | 1 linear, 10-12 kb | V, I |

^{a}Number of segments, conformation, size.
^{b}V, vertebrate; P, plant, I, insect; F, fungus.
ND, not determined.

TABLE 2
Members of the Flaviviridae
Flaviviruses

| Antigenic Group (#, +^{a} vector^{b}) | Type members |
|---|---|
| Tick-borne encephalitis (12, T) | Central European encephalitis (TBE-W) |
| | Far Eastern encephalitis (TBE-FE) |
| Rio Bravo (6, T^{c}) | Rio Bravo |
| Japanese encephalitis (10, M) | Japanese encephalitis (JE) |
| | Kunjin (KUN) |
| | Murray Valley encephalitis (MVE) |
| | St. Louis encephalitis (SLE) |
| | West Nile (WN) |
| Tyuleniy (3, T) | Tyuleniy |
| Ntaya (5, M^{c}) | Ntaya |
| Uganda S (4, M) | Uganda S |
| Dengue (4, M) | Dengue type 1 (DEN-1) |
| | Dengue type 2 (DEN-2) |
| | Dengue type 3 (DEN-3) |
| | Dengue type 4 (DEN-4) |
| Modoc (5, U) | Modoc |
| Ungrouped (17, M^{c}) | Yellow fever (YF) |

| | |
|---|---|
| Species | Type member |

Pestiviruses

| | |
|---|---|
| Bovine viral diarrhea virus 1 (BVDV-1) | BVDV strain NADL |
| Bovine viral diarrhea virus 2 (BVDV-2) | BVDV strain 890 |
| Hog cholera or classical swine fever virus | CSFV Alfort/187 |
| (CSFV^{d}) | |
| Border disease virus (BDV) | BDV BD31 |

Hepaciviruses

| | |
|---|---|
| Hepatitis C virus (HCV)^{e} | HCCV-1 |

Unassigned^{f}

| Group | Type member |
|---|---|
| GB virus-A-like viruses | GB virus-A (GBV-A) |
| GB virus-B | GB virus-B (GBV-B) |
| GB virus-C | GB virus-C (GBV-C, HGV^{g}) |

^{a}Number of recognized members in each antigenic group.
^{b}Arthropod vectors: T, tick; M, mosquito; U, unidentified or no vector.
^{c}Arthropod vectors for some members of these groups have not been identified. The ungrouped flaviviruses include mosquito- and tick-transmitted viruses as well as some with no known vector.
^{d}In the pestivirus literature, HCV has been a common abbreviation for hog cholera virus. More recent publications and this chapter use CSFV to avoid confusion with the human hepatitis C viruses.
^{e}The hepatitis C viruses include a large number of isolates, which can be divided into six major genotypes and over 100 subtypes on the basis of genetic divergence.
^{f}Several animal and human viruses most closely related to HCV have recently been described. These viruses have been tentatively assigned to the Flaviviridae based on their genomic organization and genetic similarity to recognized members of the family.
^{g}GBV-C and hepatitis G virus (HGV) refer to the same viral agent. Currently, it is unclear if this prevalent human virus is associated with clinical disease.

### Classification:

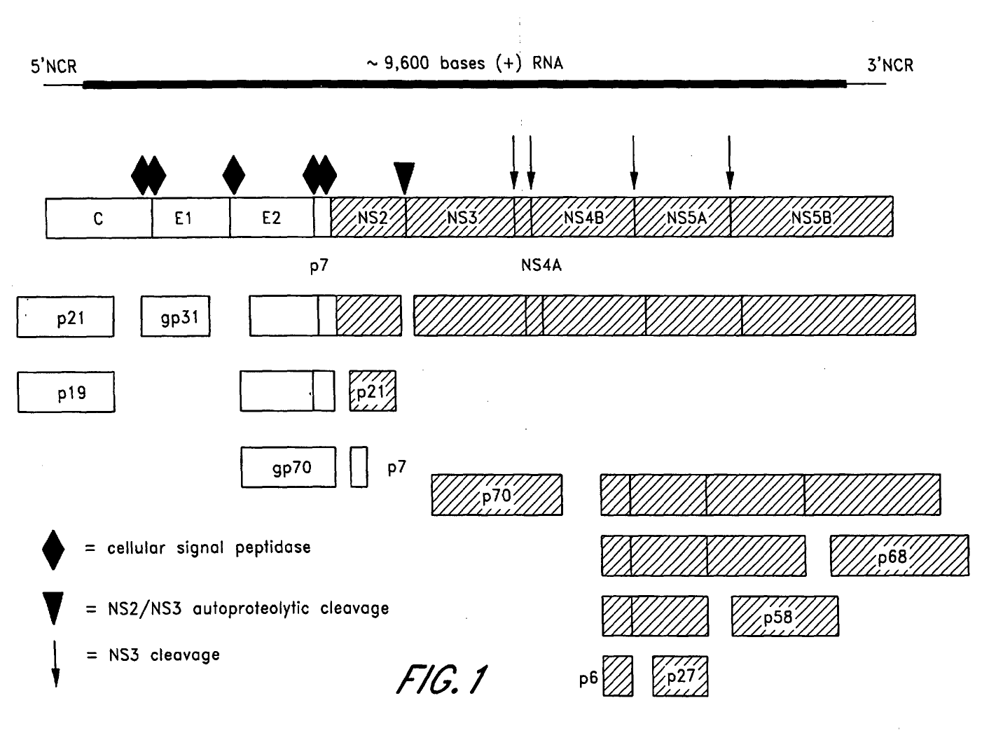

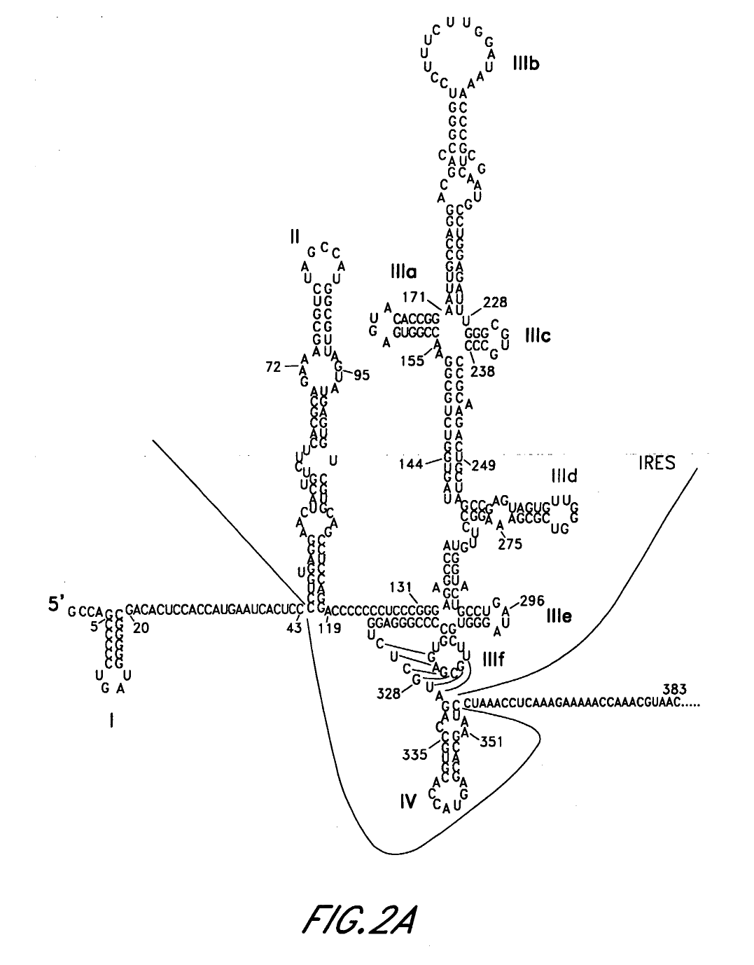

HCV has a similar genomic organization and polyprotein hydrophobicity profile as the pestiviruses and flaviviruses and has been classified as a separate genus in the family Flaviviridae. The HCV viral particle is about 50 nm in diameter and consists of an envelope derived from host membranes into which are inserted the virally encoded glycoproteins (E1 and E2) surrounding a nucleocapsid and a positive-sense, single-stranded RNA genome of about 9,500 nucleotides. The genome contains highly conserved untranslated regions (UTRs) at both the 5′ and 3′ termini, which flank a single ORF encoding a polyprotein of 3,000 amino acids. This is processed cotranslationally and posttranslationally by cellular and viral proteases to produce the specific viral gene products outlined in FIG. 1. Referring to Table 3, the structural proteins, core, E1 and E2, are located in the N-terminal quarter, with the nonstructural (NS) proteins (NS2, NS3, NS4A, NS4B, NS5A and NS5B) in the remaining portion of the polyprotein.

### The 5′-Untranslated Region

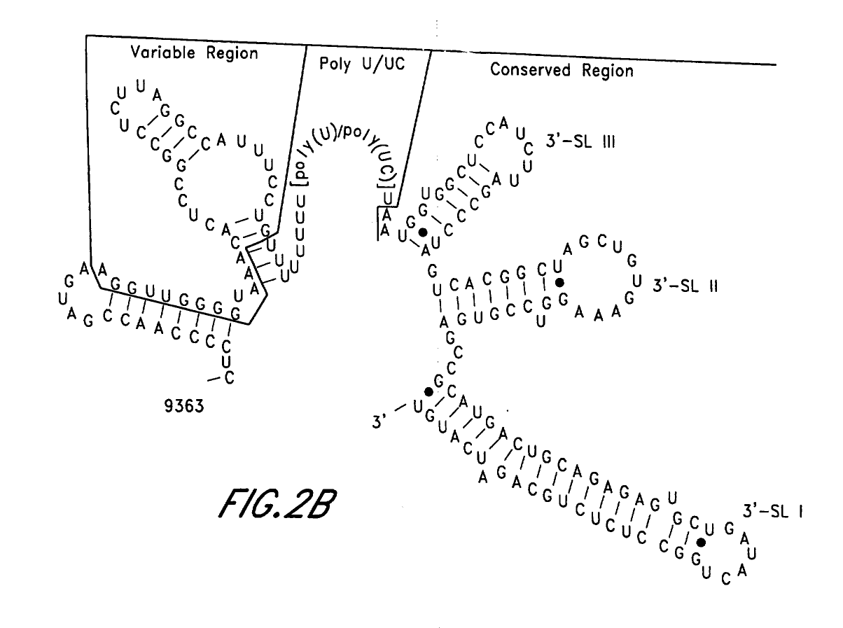

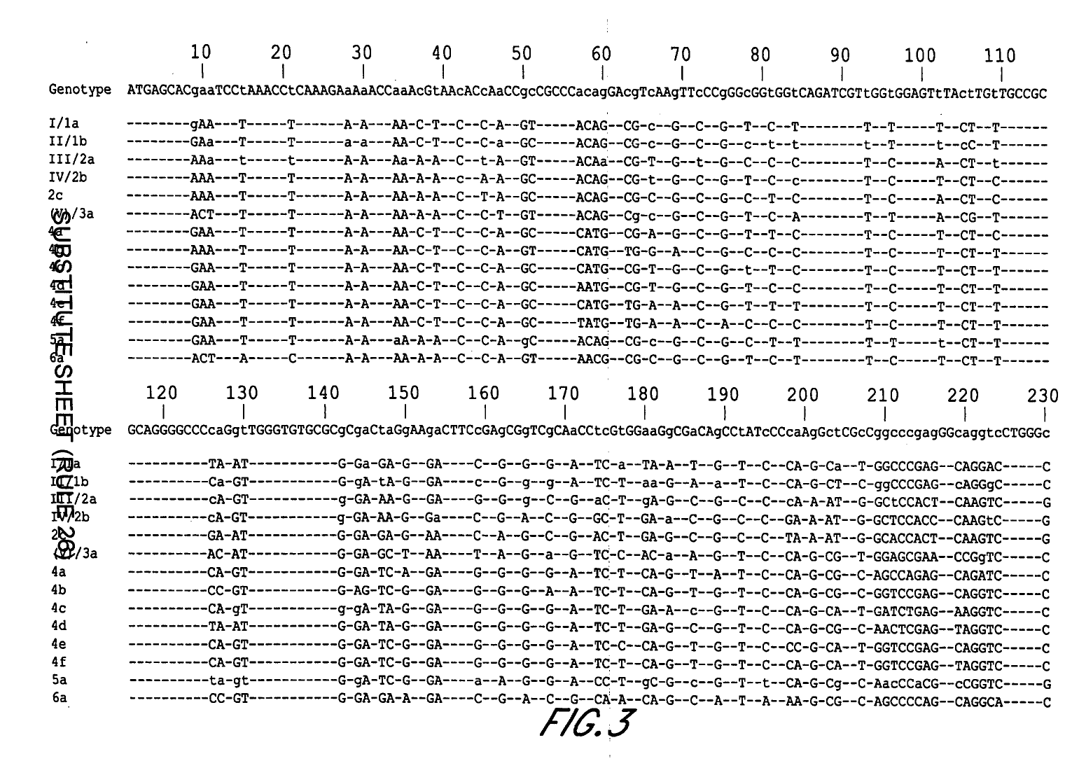

The complete 5′-UTR consists of 341 nucleotides, the proposed secondary structure of which is shown in FIG. 2A. This is one of the most conserved portions of the HCV genome, and while nucleotide variations characteristic of different HCV types exist and have been used in PCR-based genotyping assays, the overall secondary structure is still conserved. Several groups have confirmed an internal ribosome entry site (IRES) that can direct translation in a cap-independent manner. This occupies most of the HCV 5′-UTR, as depicted in FIG. 2A, with a requirement for sequence downstream of the initiator AUG for efficient IRES function. In the genome, coding sequence from the core region of 12 to 30 nucleotides is sufficient for the IRES function, but some reporter gene sequences can be substituted. It has been suggested that specific sequences in this region downstream of the initiating AUG contribute to IRES function through RNA-RNA interaction. The 5′-UTRs from different genotypes have been shown to direct translation with different efficiencies, which may be dependent on subtle sequence variations within this region affecting RNA-RNA interactions or RNA-protein interactions. Several cellular proteins have been shown to bind the 5′-UTR of HCV and play functional roles in HCV internal initiation, including polypyrimidine tract-binding protein and the eukaryotic translation initiation factor eIF3.

### The 3′-Untranslated Region

After the ORF stop codon is the 3′-UTR. This contains, in the 5′ to 3′ direction, a region of about 30 nucleotides, which shows nucleotide variability between genotypes, a poly(U) tract of variable length, a polypyrimidine C(U)ₙ stretch, and a highly conserved 98-base sequence, thought to represent the 3′-terminus of the genome. Computer predictions of the secondary structure and cleavage analyses show that the region can form stable stem-loops (see FIG. 2B), although the upstream two stem-loops have not been confirmed experimentally. Specific interactions between the 3′-UTR, in particular the conserved 98 base region at the 3′-terminus, and cellular proteins, including polypyrimidine tract-binding protein suggest that this region is involved in viral replication and possibly translation. Studies in chimpanzees using infectious clones of HCV deletion mutants have shown that the poly(U/UC) region and the conserved 98 base region are critical for infectivity but that the variable region, or its secondary structure, is not. This further confirms the involvement of the proximal 3′ region of the HCV genome in replication.

TABLE 3
Features of the HCV genome and polyprotein products

| Genome region, | Nucleotide | AA location of the mature | Approximate size | |
|---|---|---|---|---|
| protein | location^{a} | product in the HCV polyprotein^{a} | by SDS PAGE | Functions |
| 5′ UTR | 1-341 | | None^{b} | Initiation of translation, replication^{d} |
| C | 342-857 | 1-191/179/182 | p21/p19 | Structural, encapsidation of viral RNA^{d} |
| E1 | 915-1490 | 192-383 | gp31^{c} | Structural, receptor binding, cell entry^{d} |
| E2 | 1491-2579 | 384-746 | gp70^{c} | Structural, receptor binding, cell entry^{d} |
| E2-p7 | 1491-2768 | 384-809 | gp70^{c} | NK, possible precursor or structural function |
| p7 | 2580-2768 | 747-809 | p7 | NK |
| NS2 | 2769-3419 | 810-1026 | p21 | Part of NS2-3 protease |
| NS3 | 3420-5312 | 1027-1657 | p70 | Part of NS2-3 protease, serine protease, helicase, |
| | | | | NTPase |
| NS4A | 5313-5476 | 1658-1711 | p6 | Cofactor for NS3 serine protease activity |
| NS4B | 5477-6257 | 1712-1972 | p27 | Replicase component^{d} |
| NS5A | 6258-7600 | 1973-2420 | p58 | Replicase component^{d} |
| NS5B | 7601-9374 | 2421-3011 | p68 | RNA-dependent RNA polymerase |
| 3′ UTR | 9375-~9621 | | None | Replication,^{d} packaging of viral genome^{d} |

^{a}Based on HCV-H strain nucleotide and amino acid sequence.
^{b}5′ UTR contains several short open reading frames (ORFs), whether there is production of polypeptides or their possible functions is unknown.
^{c}Indicates proteins are N-glycosylated.
^{d}Designates putative function based on comparisons with other viruses.
AA, amino acid; SDS PAGE, sodium dodecyl sulfate polyacrylamide electrophoresis; NTPase, nucleoside triphosphatase; NK, not known.

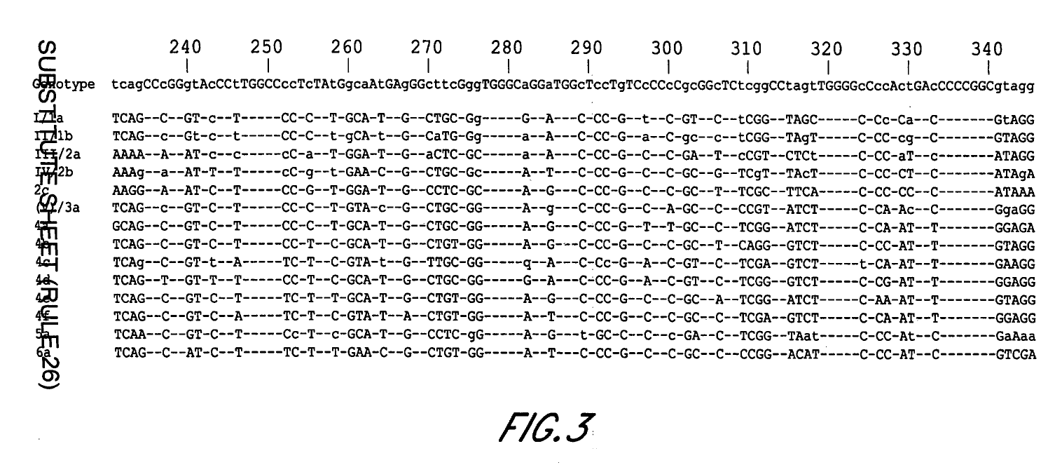

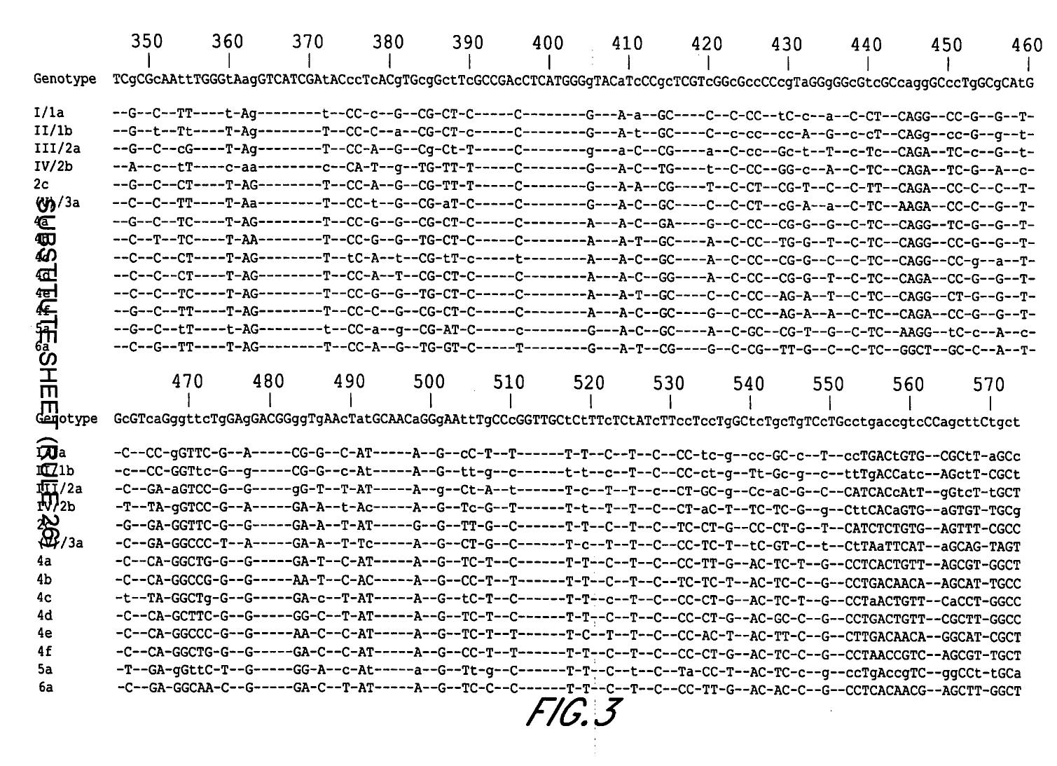

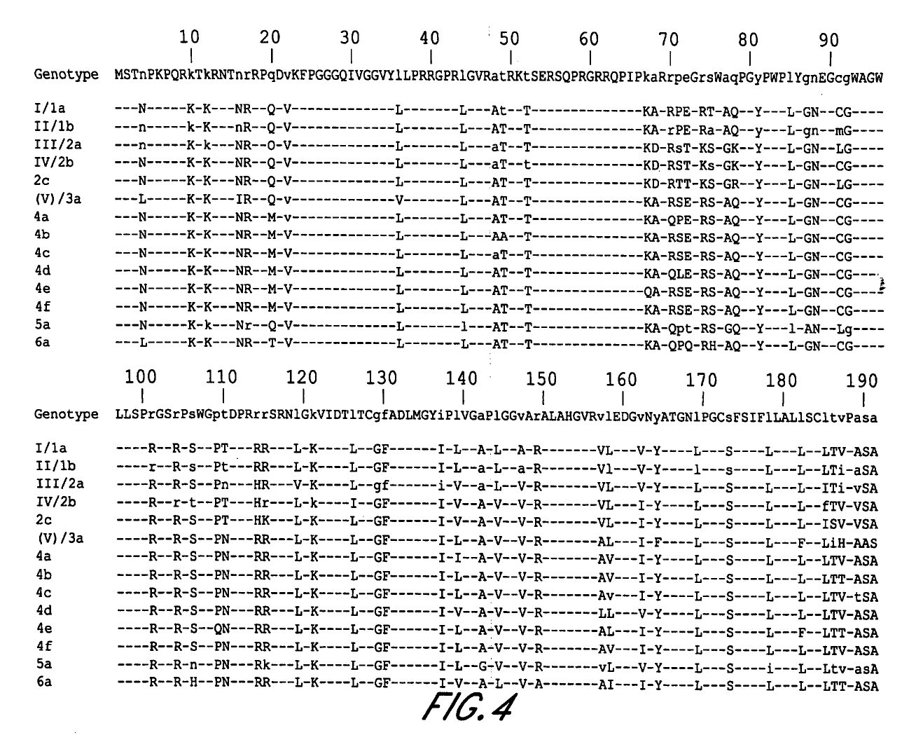

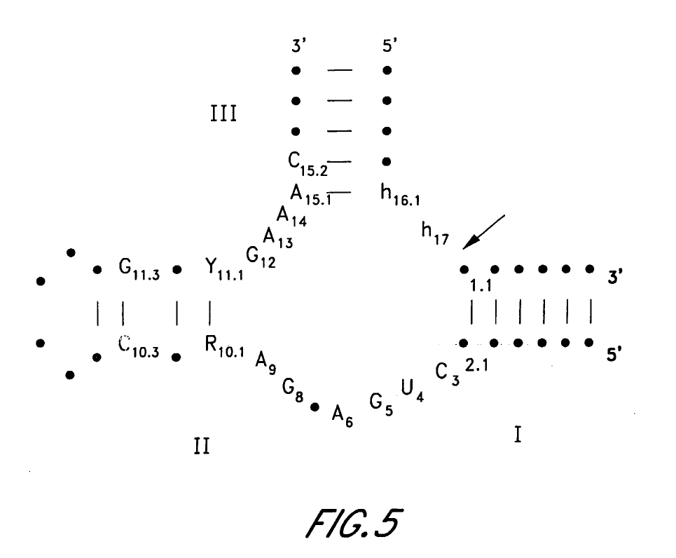

In addition to genotype 1b, the invention is applicable to other genotypes of HCV. Referring to FIGS. 3 and 4, Bukh et al (1994 PNAS USA 91:8239-8243) provided evidence for the existence of at least 6 major genetic groups consisting of at least 14 minor genotypes of HCV (i.e., genotypes I/1a, H/1b, III/2a, IV/2b, 2c, V/3a, 4a-4-f, 5a, and 6a). The sequence reported in that paper have been deposited in the GenBank data base (accession nos. U10189-U10240).

### Authentic HCV Nucleic Acid Sequences

In one embodiment, the present invention advantageously provides an authentic hepatitis C virus (HCV) nucleic acid, e.g., DNA or RNA, sequence. A functional HCV nucleic acid of the invention advantageously provides for in vitro production of HCV virions. Despite arduous efforts, in vitro production of HCV virions has not previously been successful, thus precluding systematic evaluation of the virus's mechanisms of replication, development of antiviral therapeutic agents using in vitro assay systems, and development of sensitive in vitro diagnostic assay systems. In addition, the sequences of the invention now enable in vitro production of HVC virions and virus particle proteins under conditions that permit proper processing, and thus expression of proteins that bear the closest possible structural resemblance to native HCV. Such HCV virions and virus particle proteins are preferred for anti-HCV vaccine development.

The present invention is based, in part, on generation of a functional genotype 1b cDNA clone, which can be used as a basis for preparation of functional clones for other HCV genotypes (e.g., constructed and verified using similar methods). These products have a variety of applications for development of (i) more effective HCV therapies; (ii) HCV vaccines; and (iii) HCV diagnostics. Examples of these applications are described below.

The current invention describes the preparation of an HCV genetic sequence and the use of this information to construct full-length HCV cDNA clones capable of yielding replication-competent RNA transcripts. The rigorous generation of terminal sequences, including the provision of highly conserved sequences at the 5′ and 3′ ends, the use of methods for assembling HCV cDNA clones, and the assembly of clones reflecting a genetic sequence, all contributed to the success of the present invention.

The term “authentic” is used herein to refer to an HCV nucleic acid, whether a DNA (e.g., cDNA) or RNA, that provides for full genomic replication and production of functional HCV proteins, or components thereof. In a specific embodiment, an authentic HCV nucleic acid is infectious, e.g., in a chimpanzee model or in tissue culture, forms viral particles (i.e., virions), or both. However, an authentic HCV nucleic acid of the invention may also be attenuated, such that it only produces some (not all) functional HCV proteins, or it can productively infect cells without replication in the absence of a helper cell line or plasmid, etc. The authentic HCV exemplified in the present application contains all of the virus-encoded information, whether in RNA elements or encoded proteins, necessary for initiation of an HCV replication cycle that corresponds to replication of wild-type virus in vivo. The specific HCV clones described herein, including the embodiment 1b and variants thereof described or exemplified in this application, represent a preferred starting material for developing HCV therapeutics, vaccines, and diagnostics. In particular, use of the HCV nucleic acids of the invention assures that authentic HCV components are involved, since, unlike the cloned HCVs of the prior art, these components together provide a HCV virion. The specific starting materials described herein, and preferably the 1b plasmid clone harboring authentic HCV cDNA, can be modified as described herein, e.g., by site-directed mutagenesis, to produce an attenuated derivative. Alternatively, sequences from other genotypes or isolates can be substituted for the homologous sequence of the specific embodiments described herein. For example, an authentic HCV nucleic acid of the invention may comprise the genetic 5′ and 3′ sequences disclosed herein, e.g., on a recipient plasmid, and a polyprotein coding region from another isolate or genotype is substituted for the homologous polyprotein coding region of the HCV exemplified herein. In addition, the general characteristics for an authentic HCV as described herein, including but not limited to containing 5′ or 3′ sequences, or both, containing an ORF that encodes a polyprotein whose cleavage products form functional components of HCV virus particles and RNA replication machinery, and, in a preferred embodiment, incorporate a genetic sequence of a specific isolate or genotype provide for obtaining authentic HCV clones.

The term “genetic sequence” is used herein to refer to a functional HCV genomic sequence, or any portion thereof, including the 5′-UTR, polyprotein coding sequence or portion thereof, and 3′-UTR, which is obtained by reproducing the HCV residues of an independent clone of a strain or genotype of HCV or is determined by identifying the consensus residues from three or more independent clones of a strain or genotype of HCV.

The authentic HCV nucleic acid of the invention preferably includes a 5′-UTR sequence.

In an authentic HCV nucleic acid of the invention, the 3′-UTR comprises a polypyrimidine region. In positive-strand HCV RNA, the region corresponds to a poly(U)/poly(UC) tract. Naturally, in positive-strand HCV DNA, this is a poly(T)/poly(TC) tract. An authentic HCV nucleic acid of the invention may have a variable length polypyrimidine tract.

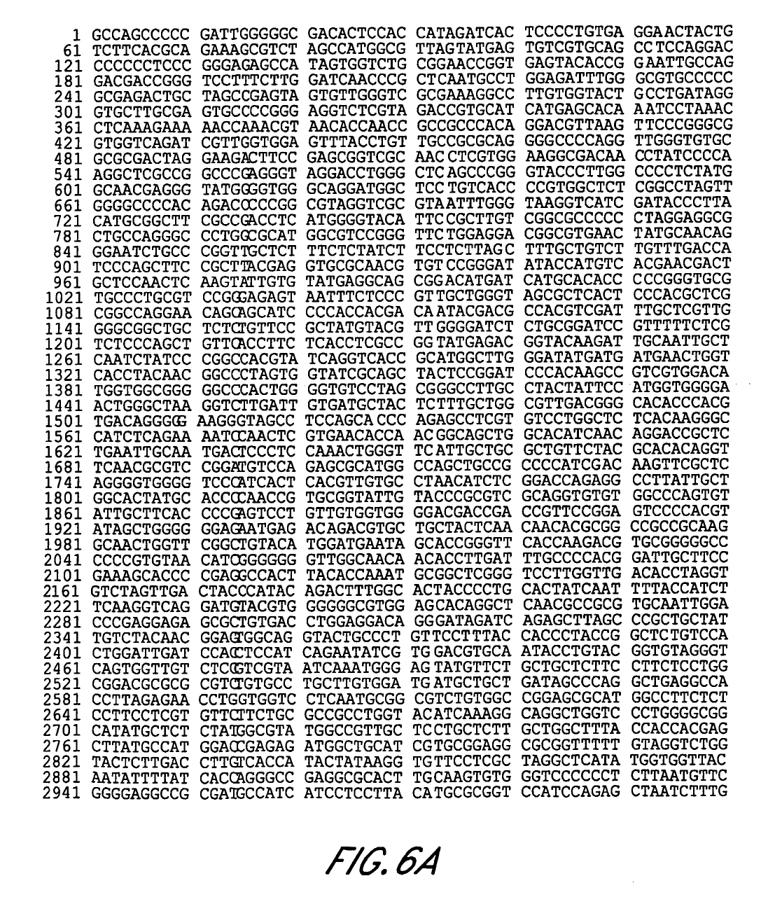

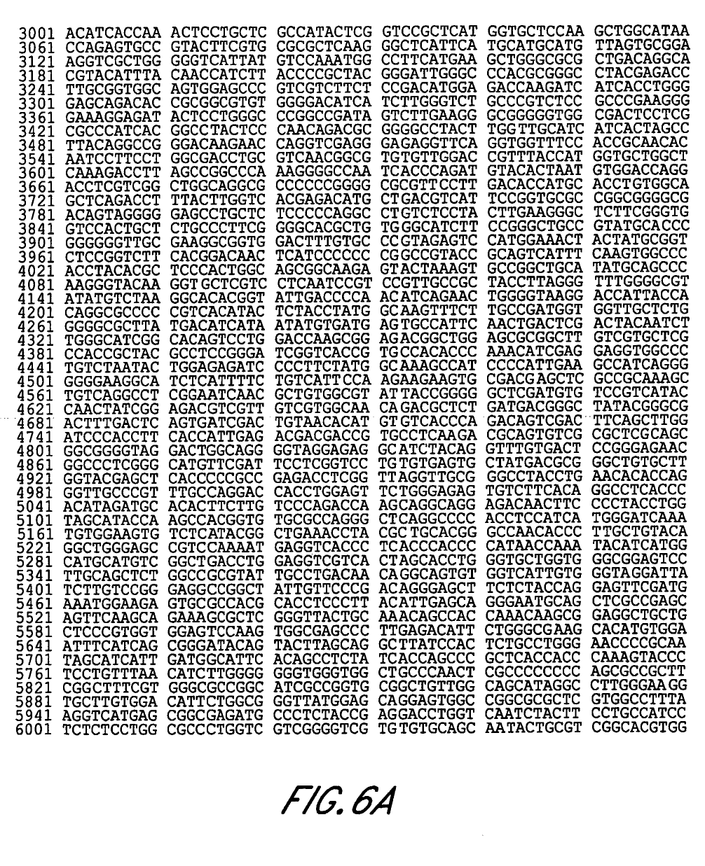

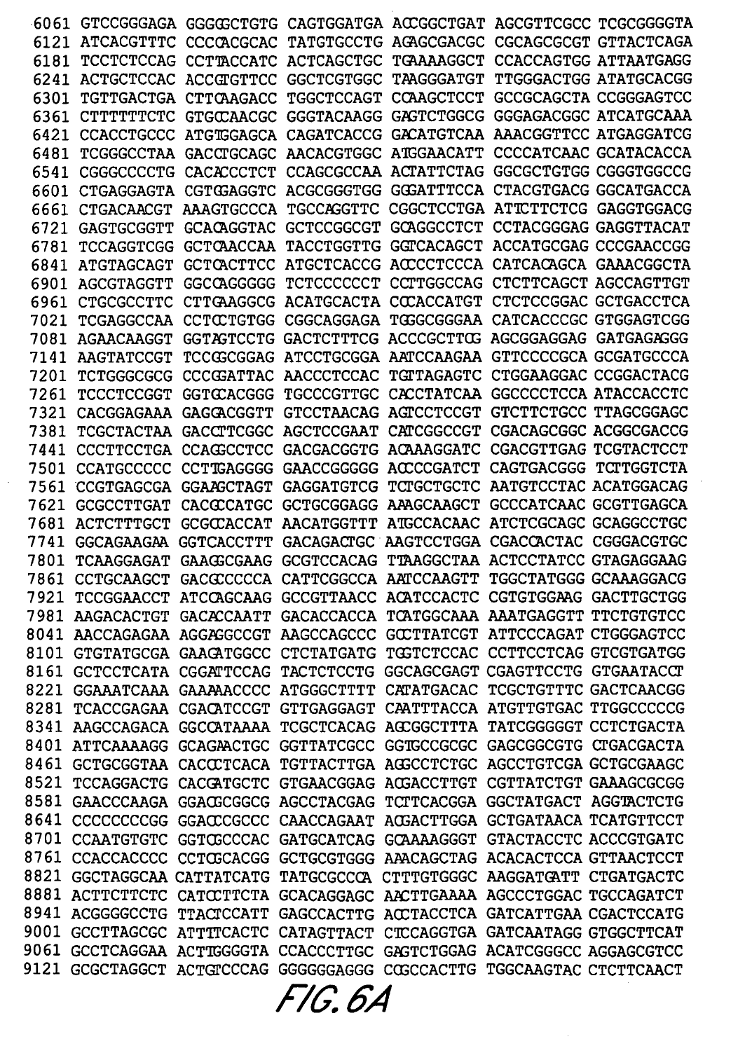

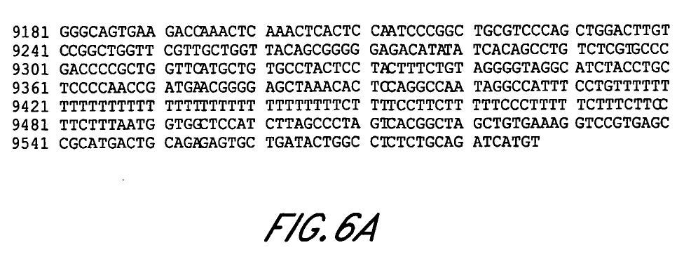

In a specific embodiment of the invention, the cDNA encoding a replication-competent RNA transcript possesses the full-length sequence as shown in GenBank accession number AF333324, referenced in Thomson et al. 2001 Gastroenterology 121:1226, and illustrated in FIG. 6.

Various terms are used herein, which have the following definitions:

The phrase “pharmaceutically acceptable” refers to molecular entities and compositions that are physiologically tolerable and do not typically produce an allergic or similar untoward reaction, such as gastric upset, dizziness and the like, when administered to a human.

Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host.

The term “adjuvant” refers to a compound or mixture that enhances the immune response to an antigen. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic, polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

In a specific embodiment, the term “about” or “approximately” means within 20%, preferably within 10%, and more preferably within 5% of a given value or range.

The following subsections of the application, which further amplify the foregoing disclosure, are provided for convenience and not by way of limitation.

### Functional Full-Length Clones for Other HCV Isolates and Genotypes

Using the approaches described here, functional full-length clones for the other HCV genotypes can be built and utilized for biological studies and antiviral screening and evaluation. In this extension of the invention, libraries can be constructed using RNA from single-exposure patients with high RNA titers (greater than 10⁶/ml) and known clinical history. A HCV or consensus sequence for the isolate can be generated from the sequences of individual clones in the library. New recipient plasmids containing a promoter, 5′ and 3′ terminal HCV or consensus sequences, and an open reading frame can be constructed.

In one embodiment, the present invention contemplates isolation of other HCV genomic sequences, or consensus genomic sequences. In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition Cold Spring Harbor Laboratory Press, 1989; F. M. Ausubel et al. (eds.), Current Protocols in Molecular Biology, John Wiley & Sons, Inc., 1989.

Therefore, if appearing herein, the following terms shall have the definitions set out below.

It should be appreciated that the terms HCV sequence, such as the “3′ terminal sequence element,” “3′ terminus,” “3′ sequence element,” are meant to encompass all of the following sequences: (i) an RNA sequence of the positive-sense genome RNA; (ii) the complement of this RNA sequence, i.e., the HCV negative-sense RNA; (iii) the DNA sequence corresponding to the positive-sense sequence of the RNA element; and (iv) the DNA sequence corresponding to the negative-sense sequence of the RNA element. Accordingly, nucleotide sequences displaying substantially equivalent or altered properties are likewise contemplated. These modifications may be deliberate, for example, such as modifications obtained through site-directed mutagenesis, or may be accidental, such as those obtained through mutations in hosts that are producers of the complex or its named subunits.

A “construct” is a replicon, such as a plasmid, phage, or cosmid, to which another DNA (or RNA) segment may be joined so as to bring about the replication of the attached segment. A “cassette” refers to a segment of DNA or RNA that can be inserted into a vector at specific restriction sites. The segment of DNA or RNA encodes a polypeptide or RNA of interest, and the cassette and restriction sites are designed to ensure insertion of the cassette in the proper reading frame for transcription and translation.

Transcriptional and translational control sequences are DNA or RNA regulatory sequences, such as promoters, enhancers, polyadenylation signals, terminators, IRES elements, and the like, that provide for the expression of a coding sequence in a host cell. A coding sequence is “under the control of” or “operably (also operatively) associated with” transcriptional and translational control sequences in a cell when RNA polymerase transcribes the coding sequence into RNA. RNA sequences can also serve as expression control sequences by virtue of their ability to modulate translation, RNA stability, RNA replication, and RNA transcription (for RNA viruses).

A “promoter sequence” is a DNA or RNA regulatory region capable of binding RNA polymerase in a cell and initiating transcription of a downstream (3′ direction) coding or noncoding sequence. Thus, promoter sequences can also be used to refer to analogous RNA sequences or structures of similar function in RNA virus replication and transcription. Preferred promoters for or bacterial expression of infections HCV DNA clones of the invention are the phage promoters T7, T3, and SP6. Alternatively, a nuclear promoter, such as cytomegalovirus immediate-early promoter, can be used. Indeed, depending on the system used, expression may be driven from a eukaryotic, prokaryotic, or viral promoter element. Promoters for expression of HCV RNA can provide for capped or uncapped transcripts.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.). Such proteins (and their encoding genes) have a high degree of sequence similarity. The term “sequence similarity” in all its grammatical forms refers to the degree of identity or correspondence between nucleic acid or amino acid sequences of proteins that may or may not share a common evolutionary origin. However, in common usage and in the instant application, the term “homologous,” when modified with an adverb such as “substantially” or “highly,” may refer to sequence similarity and not a common evolutionary origin.

In a specific embodiment, two DNA or RNA sequences are “homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook et al., 1989, supra.

Similarly, in a particular embodiment, two amino acid sequences are “homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The term “corresponding to” in relation to nucleic acid or amino acid structure is used herein to refer similar or homologous sequences, whether the exact position is identical or different from the molecule to which the similarity or homology is measured. A nucleic acid or amino acid sequence alignment may include gaps. Thus, the term “corresponding to” refers to the sequence similarity or regions of homology, and not the numbering of the amino acid residues or nucleotide bases.

HCV genomic nucleic acids can be isolated from any source of infectious HCV, particularly from tissue samples (blood, plasma, serum, liver biopsy, leukocytes, etc.) from an infected human or simian, or other permissive animal species. Methods for obtaining genomic HCV clones or portions thereof are well known in the art, as described above (see, e.g., Sambrook et al., 1989, supra). Representative genotypes further include, but are by no means restricted to, other 1b isolates, 1a, 2a, 2b, 2c, 3a, 4a-4-f, 5a, 6a. (Bukh et al., 1994, supra). For many subtypes and genotypes, enough sequence data are available to design primers for RT/PCR and PCR assembly.

In the molecular cloning genomic HCV RNA or DNA, DNA fragments are generated, e.g., by reverse transcription into cDNA and PCR. These fragments may be assembled to form a full-length sequence. Preparation of many such fragments provides a combinatorial library of HCV clones. Such a library may yield an infectious clone; or the consensus sequence can be determined by comparing the sequences of all or a significant number of clones from such a library. Enough clones should be evaluated so that a majority of bases at any divergent position are identical. Thus, a consensus may be determined by analyzing the sequence of at least three clones. Naturally, the more error-prone the cloning method, the greater the number of clones that should be sequenced to yield an authentic HCV consensus sequence.

The genetic sequence can then be used to prepare an infectious HCV DNA clone. The fidelity of the resulting clones is preferably established by sequencing. However, selection can be carried out on the basis of the properties of the clone, e.g., if the clone encodes an infectious HCV RNA. Thus, successful preparation of an infectious HCV DNA clone may be detected by assays based on the physical, pathological, or immunological properties of an animal or cell culture transfected or infected with the clone. For example, cDNA clones can be selected that produce an HCV virion or virus particle protein that, e.g., has similar or identical physical-chemical, electrophoretic migration, isoelectric focusing, or nonequilibrium pH gel electrophoresis behavior, proteolytic digestion maps, or antigenic properties as known for native HCV or HCV virus particle proteins.

Components of functional HCV cDNA clones. Components of the functional HCV cDNA described in this invention can be used to develop cell culture-based screening assays for known or newly identified HCV antiviral targets as described infra. Examples of known or suspected targets and assays include (see Fields Virology, 2001, supra, at Ch. 34 for review), but are not limited to, the following:

The highly conserved 5′ UTR, which contains elements essential for translation of the incoming HCV genome RNA, is one target. Another target is the HCV C (capsid or core) protein. The E1, E2, and E2-p7 glycoproteins, which form the components of the virion envelope, are targets. The NS2-3 autoprotease is a further target. The NS3 serine protease and NS4A cofactor, which form a complex and mediate cleavages in the HCV polyprotein, is yet another suitable target. Other targets include the NS3 RNA-stimulated NTPase and RNA helicase. The NS5A protein, another presumed replication component, is a further target. The NS5B, which is the RNA-dependent RNA polymerase, is another target. Other targets include structural or nonstructural protein functions important for HCV RNA replication and/or modulation of host cell function. The 3′ UTR, especially the highly conserved elements (poly (U/UC) tract; 98-base terminal sequence) can be targeted.

The functional HCV cDNA clones encode all of the viral proteins and RNA elements required for RNA packaging. These elements can be targeted for development of antiviral compounds.

Due to the degeneracy of nucleotide coding sequences, other DNA sequences that encode substantially the same amino acid sequence as an HCV polyprotein coding region may be used in the practice of the present invention. These include but are not limited to homologous genes from other species, and nucleotide sequences comprising all or portions of HCV polyprotein genes altered by the substitution of different codons that encode the same amino acid residue within the sequence, thus producing a silent change. Such silent changes permit creation of genomic markers, which can be used to identify a particular infectious isolate. Likewise, the HCV genomic derivatives of the invention include, but are not limited to, those containing, as a primary amino acid sequence, all or part of the amino acid sequence of an HCV polyprotein including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Moreover, since HCV lacks proofreading activity, the virus itself readily mutates, forming mutant “quasi-species” of HCV that are also contemplated as within the present invention. Such mutations are easily identified by sequencing isolates from a subject, as detailed herein.

The clones encoding HCV derivatives and analogs of the invention can be produced by various methods known in the art. The manipulations that result in their production can occur at the gene or protein level. For example, the cloned HCV genome sequence can be modified by any of numerous strategies known in the art (Sambrook et al., 1989, supra). The genomic sequence can be cleaved at appropriate sites with restriction endonuclease(s), followed by further enzymatic modification if desired, isolated, and ligated in vitro. Alternatively, genomic fragments can be joined, e.g., with PCR, to create an HCV genome. In the production of the genomic nucleic acid derivative or analog of HCV, care should be taken to ensure that the modified genome remains within the same translational reading frame as the native HCV genome, uninterrupted by translational stop signals, in the region where the desired activity is encoded.

The HCV polyprotein-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Preferably, such mutations provide for modification of the functional activity of the HCV, e.g., to attenuate viral activity, as set forth infra. Any technique for mutagenesis known in the art can be used, including but not limited to, in vitro site-directed mutagenesis. PCR techniques are preferred for site directed mutagenesis (see PCR Technology: Principles and Applications for DNA Amplification, H. Erlich, ed., Stockton Press, 1989).

Adaptation of HCV for more efficient replication in cell culture. The engineering of dominant selectable markers under the control of the HCV replication machinery can be used to select for adaptive mutations in the HCV replication machinery. Such adaptive mutations could be manifested, but are not restricted to: (i) altering viral products responsible for deleterious effects on host cells; (ii) increasing or decreasing HCV RNA replication efficiency; (iii) increasing or decreasing HCV RNA packaging efficiency and/or assembly and release of HCV particles. Even if the sequence of an HCV original cDNA clone is incompatible with establishing replication in a particular cell type, mutations occurring during in vitro transcription, during the initial stages of HCV-mediated RNA synthesis, or incorporated in the template DNA by a variety of chemical or biological methods, supra, may allow replication in a particular cellular environment. The engineered dominant selectable marker, whose expression is dependent upon productive HCV RNA replication, can be used to select for adaptive mutations in either the HCV replication machinery or the transfected host cell, or both.

Chimeric HCV clones. Components of these functional clones can also be used to construct chimeric viruses for assay of HCV gene functions and inhibitors thereof. In one such extension of the invention, functional HCV elements such as the 5′ IRES, proteases, RNA helicase, polymerase, or 3′ UTR are used to create chimeric derivatives of flavivirus or pestivirus whose productive replication is dependent on one or more of these HCV elements. Such flavivirus or pestivirus HCV chimeras can then be used to screen for and evaluate antiviral strategies against these functional components.

In addition, dominant selectable markers can be used to select for mutations in the HCV replication machinery that allow higher levels of RNA replication or particle formation. In one example, engineered HCV derivatives expressing a mutant form of DHFR can be used to confer resistance to methotrexate (MTX). As a dominant selectable marker, mutant DHFR is inefficient since nearly stoichiometric amounts are required for MTX resistance. By successively increasing concentrations of MTX in the medium, increased quantities of DHFR will be required for continued survival of cells harboring the replicating HCV RNA. This selection scheme, or similar ones based on this concept, can result in the selection of mutations in the HCV RNA replication machinery allowing higher levels of HCV RNA replication and RNA accumulation. Similar selections can be applied for mutations allowing production of higher yields of HCV particles in cell culture. Such selection schemes involve harvesting HCV particles from culture supernatants or after cell disruption and selecting for MTX-resistant transducing particles by reinfection of naive cells.

The identified and isolated genomic RNA can be reverse transcribed into its cDNA. cDNA could also be made by “long” PCR to include the promoter, or by using 3′-terminal sequence-specific primers for insertion in an appropriate recipient vector. Any of these cDNAs may be inserted into an appropriate cloning vector, e.g., which comprises 5′- and 3′-UTRs, along with a suitable promoter. A clone that includes a promoter can be used directly for production of functional HCV RNA. A large number of vector-host systems known in the art may be used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pTET, etc. The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transduction, transformation, transfection, infection, electroporation, etc., so that many copies of the gene sequence are generated.

### Expression of HCV RNA and Polypeptides

The HCV DNA, which codes for HCV RNA and HCV proteins, particularly HCV RNA replicase or virion proteins, can be inserted into an appropriate expression vector, i.e., a vector that contains the necessary elements for the transcription and translation of the inserted protein-coding sequence. Such elements are termed herein a “promoter.” Thus, the HCV DNA of the invention is operationally (or operably) associated with a promoter in an expression vector of the invention. An expression vector also preferably includes a replication origin. The necessary transcriptional and translational signals can be provided on a recombinant expression vector.

Potential host-vector systems include but are not limited to mammalian cell systems infected with recombinant virus (e.g., vaccinia virus, adenovirus, Sindbis virus, Semliki Forest virus, etc.); insect cell systems infected with recombinant viruses (e.g., baculovirus); microorganisms such as yeast containing yeast vectors; plant cells; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The cell into which the recombinant vector comprising the HCV DNA clone has been introduced is cultured in an appropriate cell culture medium under conditions that provide for expression of HCV RNA or such HCV proteins by the cell. Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of HCV RNA or protein may be controlled by any promoter/enhancer element known in the art, but these regulatory elements must be functional in the host selected for expression. Promoters that may be used to control expression include, but are not limited to, the SV40 early promoter region, the promoter contained in the 3′ long terminal repeat of Rous sarcoma virus, the herpes thymidine kinase promoter, the regulatory sequences of the metallothionein gene; prokaryotic expression vectors such as the β-lactamase promoter, or the tac promoter; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region, which is active in pancreatic acinar cells; insulin gene control region, which is active in pancreatic beta cells, immunoglobulin gene control region, which is active in lymphoid cells, mouse mammary tumor virus control region, which is active in testicular, breast, lymphoid and mast cells, albumin gene control region, which is active in liver, alpha-fetoprotein gene control region, which is active in liver, alpha 1-antitrypsin gene control region, which is active in the liver, beta-globin gene control region, which is active in myeloid cells, myelin basic protein gene control region, which is active in oligodendrocyte cells in the brain, myosin light chain-2 gene control region, which is active in skeletal muscle, and gonadotropic releasing hormone gene control region, which is active in the hypothalamus.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCRI, pBR322, pMal-C2, pET, pGEX, pMB9 and their derivatives, plasmids such as RP4; phage DNAS, e.g., the numerous derivatives of phage X, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like known in the art.

In addition to the preferred sequencing analysis, expression vectors containing an HCV DNA clone of the invention can be identified by four general approaches: (a) PCR amplification of the desired plasmid DNA or specific mRNA, (b) nucleic acid hybridization, (c) presence or absence of selection marker gene functions, (d) analysis with appropriate restriction endonucleases and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to the HCV DNA. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, e.g., HCV RNA, HCV virions, or HCV viral proteins.

For example, in a baculovirus expression systems, both non-fusion transfer vectors, such as but not limited to pVL941 (BamHI cloning site), pVL1393 (BamHI, SmaI, XbaI, EcoRI, NotI, XmaIII, BglII, and PstI cloning site; Invitrogen), pVL1392 (BglII, PstI, NotI, XmaIII, EcoRI, XbaI, SmaI, and BamHI cloning site; Invitrogen), and pBlueBacIII (BamHI, BglII, PstI, NcoI, and HindIII cloning site, with blue/white recombinant screening possible; Invitrogen), and fusion transfer vectors, such as but not limited to pAc700 (BamHI and KpnI cloning site, in which the BamHI recognition site begins with the initiation codon), pAc701 and pAc702 (same as pAc700, with different reading frames), pAc360 (BamHI cloning site 36 base pairs downstream of a polyhedrin initiation codon; Invitrogen), and pBlueBacHisA, B, C (three different reading frames, with BamHI, BglII, PstI, NcoI, and HindIII cloning site, an N-terminal peptide for ProBond purification, and blue/white recombinant screening of plaques; Invitrogen) can be used.

Examples of mammalian expression vectors contemplated for use in the invention include vectors with inducible promoters, such as the dihydrofolate reductase (DHFR) promoter, e.g., any expression vector with a DHFR expression vector, or a DHFR/methotrexate coamplification vector, such as pED (PstI, SalI, SbaI, SmaI, and EcoRI cloning site, with the vector expressing both the cloned gene and DHFR. Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamHI, SfiI, XhoI, NotI, NheI, HindIII, NheI, PvuII, and KpnI cloning site, constitutive RSV-LTR promoter, hygromycin selectable marker; Invitrogen), pCEP4 (BamHI, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamHI cloning site, inducible metallothionein IIa gene promoter, hygromycin selectable marker: Invitrogen), pREP8 (BamHI, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Regulatable mammalian expression vectors, can be used, such as Tet and rTet (Gossen and Bujard, 1992 PNAS USA 89:5547-51; Gossen et al. 1665 Science 268:1766-1769). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others.

Examples of yeast expression systems include the non-fusion pYES2 vector (XbaI, SphI, ShoI, NotI, GstXI, EcoRI, BstXI, BamHI, SacI, KpnI, and HindIII cloning sit; Invitrogen) or the fusion pYESHisA, B, C (XbaI, SphI, ShoI, NotI, BstXI, EcoRI, BamHI, SacI, KpnI, and HindIII cloning site, N-terminal peptide purified with ProBond resin and cleaved with enterokinase; Invitrogen), to mention just two, can be employed according to the invention.

In addition, a host cell strain may be chosen that modulates the expression of the inserted sequences, or modifies and processes the gene product in the specific fashion desired. Different host cells have characteristic and specific mechanisms for the translational and post-translational processing and modification (e.g., glycosylation, cleavage (e.g., of signal sequence)) of proteins. Expression in yeast can produce a glycosylated product. Expression in eukaryotic cells can increase the likelihood of “native” glycosylation and folding of an HCV protein. Moreover, expression in mammalian cells can provide a tool for reconstituting, or constituting, native HCV virions or virus particle proteins.

Furthermore, different vector/host expression systems may affect processing reactions, such as proteolytic, cleavages, to a different extent.

A variety of transfection methods, useful for other RNA virus studies, are enabled herein. Examples include microinjection, cell fusion, calcium-phosphatecationic liposomes such as lipofectin, DE-dextran, and electroporation. Scrape loading and ballistic methods may also be considered for cell types refractory to transfection by these other methods. A DNA vector transporter may be considered (see, e.g., Wu et al. 1989 J Biol Chem 264:16985-16987; Wu and Wu 1988 J Biol Chem 263:14621-14624).

### In Vitro Infection with HCV

Identification of cell lines supporting HCV replication. An important aspect of the invention is a method it provides for developing new and more effective anti-HCV therapy by conferring the ability to evaluate the efficacy of different therapeutic strategies using an authentic and standardized in vitro HCV replication system. Such assays are invaluable before moving on to trials using rare and valuable experimental animals, such as the chimpanzee, or HCV-infected human patients.

The HCV infectious clone technology can be used to establish in vitro systems for analysis of HCV replication and packaging. These include, but are not restricted to, (i) identification or selection of permissive cell types (for RNA replication, virion assembly and release); (ii) investigation of cell culture parameters (e.g., varying culture conditions, cell activation, etc.) or selection of adaptive mutations that increase the efficiency of HCV replication in cell cultures; and (iii) definition of conditions for efficient production of infectious HCV particles (either released into the culture supernatant or obtained after cell disruption). These and other readily apparent extensions of the invention have broad utility for HCV therapeutic, vaccine, and diagnostic development.

General approaches for identifying permissive cell types are outlined below. Examples of cell types potentially permissive for HCV replication include, but are not restricted to, primary human cells (e.g., hepatocytes, T-cells, B-cells, foreskin fibroblasts) as well as continuous human cell lines (e.g., HepG2, Huh7, HUT78, HPB-Ma, MT-2, MT-2C, and other HTLV-1 and HTLV-11 infected T-cell lines, Namalwa, Daudi, EBV-transformed LCLs). In addition, cell lines of other species, especially those that are readily transfected with RNA and permissive for replication of flaviviruses or pestiviruses (e.g., SW-13, Vero, BHK-21, COS, PK-15, MBCK, etc.), can be tested. Cells are transfected using a method as described supra.

For replication assays, RNA transcripts are prepared using a functional clone and a corresponding non-functional, e.g., a GND (see Examples) derivative, is used as a negative control for persistence of HCV RNA and antigen in the absence of productive replication. Cell types showing a clear and reproducible difference between the intact infectious transcript and the non-functional derivative, e.g., a GND mutant control, can be subjected to analyses to verify authentic replication. Such assays include measurement of negative-sense HCV RNA accumulation by QC-RT/PCR, Northern-blot hybridization, or metabolic labeling and single cell methods, such as in situ hybridization, in situ PCR (followed by ISH to detect only HCV-specific amplification products) and immunohistochemistry.

HCV particles for studying virus-receptor interactions. In combination with the identification of cell lines that are permissive for HCV infection and replication, defined HCV stocks produced using the infectious clone technology can be used to evaluate the interaction of the HCV with cellular receptors. Assays can be set up that measure binding of the virus to susceptible cells or productive infection, and then used to screen for inhibitors of these processes.

Identification of cell lines for characterization of HCV receptors. Cell lines permissive for HCV RNA replication, as assayed by RNA transfection, can be screened for their ability to be infected by the virus. Cell lines permissive for RNA replication but that cannot be infected by the homologous virus may lack one or more host receptors required for HCV binding and entry. Such cells provide valuable tools for (i) functional identification and molecular cloning of HCV receptors and co-receptors; (ii) characterization of virus-receptor interactions; and (iii) developing assays to screen for compounds or biologics (e.g., antibodies, SELEX RNAs) that inhibit these interactions.

Once defined in this manner, these HCV receptors serve not only as therapeutic targets but may also be expressed in transgenic animals rendering them susceptible to HCV infection. Such transgenic animal models supporting HCV replication and spread have important applications for evaluating anti-HCV drugs.

Alternative approaches for identifying permissive cell lines. Besides using the unmodified HCV RNA transcripts derived from functional clones, these functional HCV clones can be engineered to provide selectable markers for HCV replication. For instance, genes encoding dominant selectable markers can be expressed as part of the HCV polyprotein, or as separate cistrons located in permissive regions of the HCV RNA genome. Such engineered derivatives have been successfully constructed for other RNA viruses such as Sindbis virus (Frolov et al. 1996 PNAS USA 93:11371-11377) or the flavivirus Kunjin (Khromykh and Westaway, 1997 J Virol 71:1497-1505). Examples of selectable markers for mammalian cells include, but are not limited to, the genes encoding dihydrofolate reductase (DHFR; methotrexate resistance), thymidine kinase (tk; methotrexate resistance), puromycin acetyl transferase (pac; puromycin resistance), neomycin resistance (neo; resistance to neomycin or G418), mycophenolic acid resistance (gpt), hygromycin resistance, and resistance to zeocin. Strategies for functional expression of heterologous genes have been described. Examples include: (i) in-frame insertion into the viral polyprotein with cleavage(s) to produce the selectable marker protein mediated by cellular or viral proteases; (ii) creation of separate cistrons using engineered translational start and stop signals. Examples include, but are not restricted to, the use of internal ribosome entry site (IRES) RNA elements derived from cellular or viral mRNAs. In a particular manifestation, a cassette including an IRES element and an antibiotic resistance gene is inserted in the HCV 3′ UTR hypervariable region. Transcribed RNAs are used to transfect human hepatocyte or other cell lines and the antibiotic used for selecting resistant cell populations.

Alterations of the HCV cDNA can be made to produce lines expressing convenient assayable markers as indirect indicators of HCV replication. Such self-replicating RNAs constitute the entire HCV genome RNA or RNA replicons, where regions non-essential for RNA replication have been deleted. Assayable genes might include a second dominant selectable marker, or those encoding proteins with convenient assays. Examples include, but are not restricted to, β-galactosidase, β-glucuronidase, firefly or bacterial luciferase, green fluorescent protein (GFP) and humanized derivatives thereof, cell surface markers, and secreted markers. Such products are either assayed directly or may activate the expression or activity of additional reporters.

### Selection and Analysis of Drug-Resistant Variants

Cell lines supporting HCV replication can be used to examine the emergence of HCV variants with resistance to existing and novel therapeutics. Like all RNA viruses, the HCV replicase is presumed to lack proofreading activity and RNA replication is therefore error prone, giving rise to a high level of variation. The variability manifests itself in the infected patient over time and in the considerable diversity observed between different isolates. The emergence of drug-resistant variants is likely to be an important consideration in the design and evaluation of HCV mono and combination therapies. HCV replication systems of the invention can be used to study the emergence of variants under various therapeutic formulations. These might include monotherapy or various combination therapies (e.g., IFN-α, ribavirin, and new antiviral compounds). Resistant mutants can then be used to define the molecular and structural basis of resistance and to evaluate new therapeutic formulations, or in screening assays for effective anti-HCV drugs (infra).

### Screening for Anti-HCV Agents

HCV-permissive cell lines can be used to screen for novel inhibitors or to evaluate candidate anti-HCV therapies. Such therapies include, but would not be limited to, (i) antisense oligonucleotides or ribozymes or siRNAs RNAs targeted to conserved HCV RNA targets; (ii) injectable compounds capable of inhibiting HCV replication; and (iii) orally bioavailable compounds capable of inhibiting HCV replication. Targets for such formulations include, but are not restricted to, (i) conserved HCV RNA elements important for RNA replication and RNA packaging; (ii) HCV-encoded enzymes; (iii) protein-protein and protein-RNA interactions important for HCV RNA replication, virus assembly, virus release, viral receptor binding, viral entry, and initiation of viral RNA replication; (iv) virus-host interactions modulating the ability of HCV to establish chronic infections; (v) virus-host interactions modulating the severity of liver damage, including factors affecting apoptosis and hepatotoxicity; (vi) virus-host interactions leading to the development of more severe clinical outcomes including cirrhosis and hepatocellular carcinoma; and (vii) virus-host interactions resulting in other, less frequent, HCV-associated human diseases.

Evaluation of antisense and ribozyme and small interfering (si) RNA therapies. The present invention extends to the preparation of antisense nucleotides and ribozymes and siRNAs that may be tested for the ability to interfere with HCV replication. This approach utilizes antisense nucleic acid and ribozymes and siRNAs to block translation of a specific mRNA, either by masking that mRNA with an antisense nucleic acid or cleaving it with a ribozyme or degrading it with siRNAs.

Screening compound libraries for anti-HCV activity. Various natural product or synthetic libraries can be screened for anti-HCV activity in the in vitro models provided by the invention. One approach to preparation of a combinatorial library uses primarily chemical methods, of which the Geysen method (Geysen et al. 1987 J Immunologic Method 102:259-274) and the method of Fodor et al. (1991 Science 251:767-773) are examples. Furka, 1991 Int J Peptide Protein Res 37:487-493 describes methods to produce a mixture of peptides that can be tested for anti-HCV activity.

In another aspect, synthetic libraries (Needels et al. 1993 PNAS USA 90:10700-4; Ohlmeyer et al. 1993 PNAS USA 90:10922-10926), and the like can be used to screen for anti-HCV compounds according to the present invention. These references describe adaptation of the library screening techniques in biological assays.

Defined/engineered HCV virus particles for neutralization assays. The functional clones described herein can be used to produce defined stocks of HCV particles for infectivity and neutralization assays. Homogeneous stocks can be produced in the cell culture systems using various heterologous expression systems (e.g., baculovirus, yeast, mammalian cells; see supra). As described above, besides homogenous virus preparations of 1b, stocks of other genotypes or isolates can be produced. These stocks can be used in cell culture assays to define approaches capable of neutralizing HCV particle production or infectivity. Examples of such molecules include, but are not restricted to, polyclonal antibodies, monoclonal antibodies, artificial antibodies with engineered/optimized specificity, single-chain antibodies (see the section on antibodies, infra), nucleic acids or derivatized nucleic acids selected for specific binding and neutralization, small orally bioavailable compounds, etc. Such neutralizing agents, targeted to conserved viral or cellular targets, can be either genotype or isolate-specific or broadly cross-reactive. They could be used either prophylactically or for passive immunotherapy to reduce viral load and perhaps increase the chances of more effective treatment in combination with other antiviral agents (e.g., IFN-α, ribavirin, etc.). Directed manipulation of HCV infectious clones can also be used to produce HCV stocks with defined changes in the glycoprotein hypervariable regions or in other epitopes to study mechanisms of antibody neutralization, CTL recognition, immune escape and immune enhancement. These studies will lead to identification of other virus-specific functions for anti-viral therapy.

### Vaccination and Protective Immunity

There are still many unknown parameters that impact on development of effective HCV vaccines. It is clear in both man and the chimpanzee that some individuals can clear the infection. Also, 10-20% of those treated with IFN appear to show a sustained response as evidenced by lack of circulating HCV RNA. Chimpanzees immunized with subunit vaccines consisting of E1E2 oligomers and vaccinia recombinants expressing these proteins are partially protected against low dose challenges (Choo et al. 1994 PNAS USA 91:1294). The infectious clone technology described in this invention has utility not only for basic studies aimed at understanding the nature of protective immune responses against HCV, but also for novel vaccine production methods.

Active immunity against HCV can be induced by immunization (vaccination) with an immunogenic amount of an attenuated or inactivated HCV virion, or HCV virus particle proteins, preferably with an immunologically effective adjuvant. An “immunologically effective adjuvant” is a material that enhances the immune response.

Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel). A vaccine for an animal, however, may contain adjuvants not appropriate for use with humans.

An alternative to a traditional vaccine comprising an antigen and an adjuvant involves the direct in vivo introduction of DNA or RNA encoding the antigen into tissues of a subject for expression of the antigen by the cells of the subject's tissue. Such vaccines are termed herein “DNA vaccines,” “genetic vaccination,” or “nucleic acid-based vaccines.” Methods of transfection as described above, such as DNA vectors or vector transporters, can be used for DNA vaccines.

Passive immunity can be conferred to an animal subject suspected of suffering an infection with HCV by administering antiserum, neutralizing polyclonal antibodies, or a neutralizing monoclonal antibody against HCV to the patient. Although passive immunity does not confer long term protection, it can be a valuable tool for the treatment of an acute infection of a subject who has not been vaccinated. Preferably, the antibodies administered for passive immune therapy are autologous antibodies. For example, if the subject is a human, preferably the antibodies are of human origin or have been “humanized,” in order to minimize the possibility of an immune response against the antibodies. In addition, genes encoding neutralizing antibodies can be introduced in vectors for expression in vivo, e.g., in hepatocytes.

Antibodies for passive immune therapy. Preferably, HCV virions or virus particle proteins prepared as described above are used as an immunogen to generate antibodies that recognize HCV. Such antibodies include but are not limited to polyclonal, monoclonal, chimeric, single chain, Fab fragments, and a Fab expression library. Various procedures known in the art may be used for the production of polyclonal antibodies to HCV. For the production of antibody, various host animals can be immunized by injection with the HCV virions or polypeptide, e.g., as described infra, including but not limited to rabbits, mice, rats, sheep, goats, etc. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward HCV as described above, any technique that provides for the production of antibody molecules by continuous cell lines in culture may be used. These include but are not limited to the hybridoma technique originally developed by Kohler and Milstein (1975 Nature 256:495-497), as well as the human B-cell hybridoma technique, and the EBV-hybridoma technique to produce human monoclonal antibodies. In an additional embodiment of the invention, monoclonal antibodies can be produced in germ-free animals. In fact, according to the invention, techniques developed for the production of “chimeric antibodies” (Morrison et al. 1984 J Bacteriol 159:870; Neuberger et al. 1984 Nature 312:604-608; Takeda et al. 1985 Nature 314:452-454) by splicing the genes from a mouse antibody molecule specific for HCV together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies can be adapted to produce HCV-specific single chain antibodies. An additional embodiment of the invention utilizes the techniques described for the construction of Fab expression libraries (Huse et al. 1989 Science 246:1275-128) to allow rapid and easy identification of monoclonal Fab fragments with the desired specificity.

Antibody fragments that contain the idiotype of the antibody molecule can be generated by known techniques. For example, such fragments include but are not limited to: the F(ab′)₂ fragment, which can be produced by pepsin digestion of the antibody molecule; the Fab′ fragments, which can be generated by reducing the disulfide bridges of the F(ab′)₂ fragment, and the Fab fragments, which can be generated by treating the antibody molecule with papain and a reducing agent.

HCV particles for subunit vaccination. A functional cDNA clone, and similarly constructed and verified clones for other genotypes, can be used to produce HCV-like particles for vaccination. Proper glycosylation, folding, and assembly of HCV particles may be important for producing appropriately antigenic and protective subunit vaccines. Several methods can be used for particle production. They include engineering of stable cell lines for inducible or constitutive expression of HCV-like particles (using bacterial, yeast or mammalian cells), or the use of higher level eukaryotic heterologous expression systems. HCV particles for immunization may be purified from either the media or disrupted cells, depending upon their localization. Such purified HCV particles or mixtures of particles representing a spectrum of HCV genotypes, can be injected with or without various adjuvants to enhance immunogenicity.

Live-attenuated HCV derivatives. The ability to manipulate the HCV genome RNA sequence and thereby produce mutants with altered pathogenicity provides a means of constructing live-attenuated HCV mutants appropriate for vaccination. Such vaccine candidates express protective antigens but would be impaired in their ability to cause disease, establish chronic infections, trigger autoimmune responses, and transform cells. Naturally, infectious HCV virus of the invention can be attenuated, inactivated, or killed by chemical or heat treatment.

### Diagnostic Methods for Infectious HCV

Diagnostic cell lines. The invention described herein can also be used to derive cell lines for sensitive diagnosis of infectious HCV in patient samples. In concept, functional HCV components are used to test and create susceptible cell lines (as identified above) in which easily assayed reporter systems are selectively activated upon HCV infection. Examples include, but are not restricted to, (i) defective HCV RNAs lacking replicase components that are incorporated as transgenes and whose replication is upregulated or induced upon HCV infection; (ii) sensitive heterologous amplifiable reporter systems activated by HCV infection. In the first manifestation, cis RNA signals required for HCV RNA amplification flank a convenient reporter gene, such as luciferase, green fluorescent protein (GFP), β-galactosidase, or a selectable marker (see above). Expression of such chimeric RNAs is driven by an appropriate nuclear promoter and elements required for proper nuclear processing and transport to the cytoplasm. Upon infection of the engineered cell line with HCV, cytoplasmic replication and amplification of the transgene is induced, triggering higher levels of reporter expression, as an indicator of productive HCV infection.

Antibody diagnostics. In addition to the cell lines described here, HCV virus particles (virions) produced by the transfected or infected cell lines may be used as antigens to detect anti-HCV antibodies in patient blood or blood products. Because the HCV virus particles are derived from an authentic HCV genome, they are likely to have structural characteristics that more closely resemble or are identical to natural HCV virus. These reagents can be used to establish that a patient is infected with HCV by detecting seroconversion, i.e., generation of a population of HCV-specific antibodies.

Alternatively, antibodies generated to the authentic HCV products prepared as described herein can be used to detect the presence of HCV in biological samples from a subject.

### Hammerhead Ribozyme

The hammerhead ribozyme was first discovered as a catalytic motif in different plant pathogen RNAs. All hammerhead ribozymes share a characteristic secondary structure (FIG. 5). Currently, this structure is known in several plant pathogen RNAs: the genomic RNA of three different plant viroids, nine satellite RNAs, a circular RNA from cherry, and a retroviroid-like element of carnation plants. In addition, three active hammerhead domains isolated from animal RNAs have also been described: a transcript from a satellite DNA of newts, the RNA encoded in Schistosoma satellite DNA, and in a DNA satellite from Dolichopoda cave crickets. All of them are involved in the processing of long multimeric transcripts into monomersized molecules. The plant-derived ones play an essential role in the in vivo replication process of RNA genomes in which they are contained. Replication of these RNAs occurs by the rolling circle mechanism. During this process, multimeric products are generated that have to be converted into genome-length strands. It is known, for at least for 14 of these RNAs, that this involves a self-cleavage reaction catalyzed by the hammerhead domain.

Molecular modeling and kinetic analysis of the hammerhead cleavage reaction in the presence of monovalent or divalent salts support the idea that divalent metal ions are not essential for the catalytic step, although they do stabilize the structure of active ribozymes.

The minimal motif that supports catalytic activity was defined by deletion assays. The reaction catalyzed by these ribozymes proceeds via transesterification chemistry that generates 5-hydroxyl and 2′,3′-cyclic phosphate termini. The hammerhead motif most commonly used is a 35-nt-long RNA molecule, but this varies depending on the length of the substrate binding arms (FIG. 5).

The hammerhead ribozyme-substrate complex is comprised of an intramolecular helix (helix II) and two intermolecular helices generated after the substrate interaction (helix I and III). Single-stranded regions are highly conserved and contain most of the important nucleotides for optimum catalytic activity. However, substrate-binding arms can be changed to modify ribozyme specificity. Helix I shows no strict sequence requirements. Nevertheless, it has been shown that the helix I region close to the catalytic core has some influence on global ribozyme structure. The nucleotide sequence of this region defines the angle between helices II and III, contributing to active conformer formation (FIG. 5). The consensus sequence for the cleavage site has been established as 5P-NHH↓ (N=any nucleotide and H=A, C or U; ↓=the cleavage site).

The three-dimensional structure of the hammerhead ribozyme was established by two different approaches: by the X-ray diffraction spectrum of the ribozyme when co-crystallized with a DNA-substrate molecule, and by FRET techniques. The data shows that the ribozyme adopts a ‘Y’ shape in which helices II and III are co-linearly stacked with helix I adjacent to helix II.

Self-trimming ribozymes have been designed by others (Altschuler, M. et al. 1992 Gene 122:85-90; Dzianott and Bujarski 1989 PNAS USA 86:4823-4827; Ruis, J. et al 1997 BioTechniques 22:338-345). Our design is similar to the one previously described by: Taira et al. 1991 Nucleic Acids Res 19:5125-5130; Ohkawa, J. et al. 1992 Nucleic Acids Symp Ser 27:15-16; Ohkawa, J. et al. 1993 Nucleic Acids Symp Ser 29:121-122) The two cis-acting ribozymes are targeted to sites within the HCV-ribozyme construct, so that following transcription they function autocatalytically, liberating the RNA genome.

### An In Vitro Model of Hepatitis C Virion Production

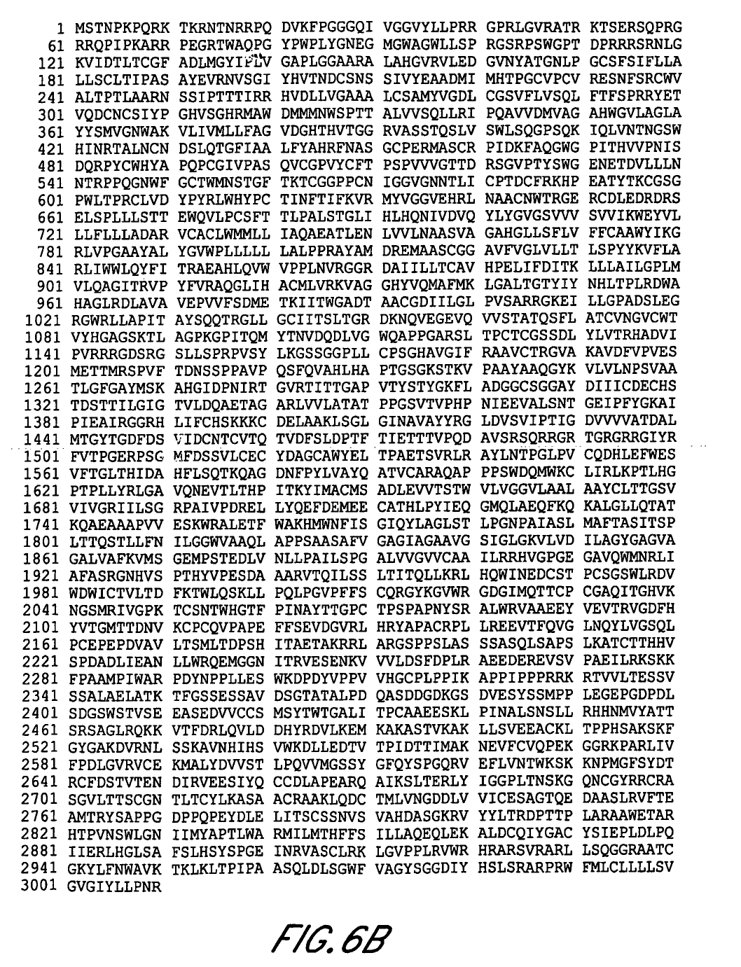

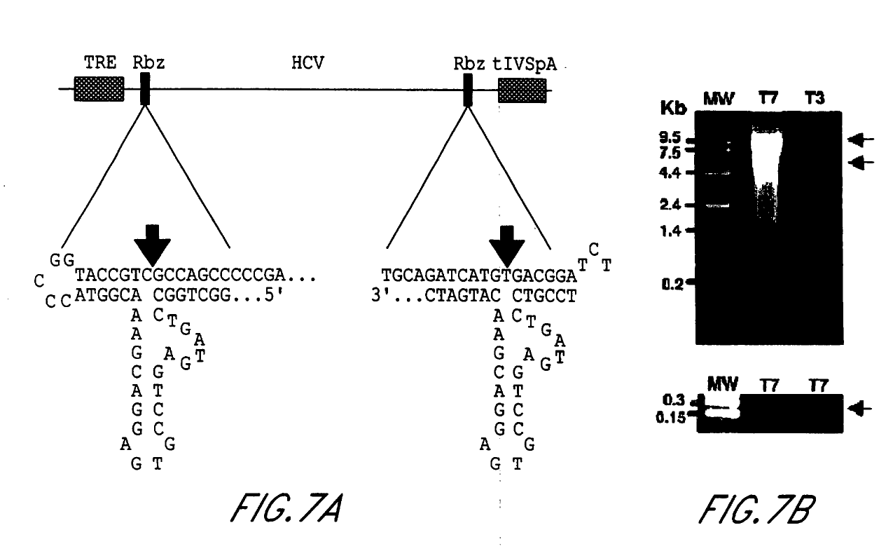

Ribozyme Activity. To prove that the ribozymes function properly in the context of HCV genome, the HCV-ribozyme RNA was generated by in vitro transcription of pHr and analyzed by formamide gel electrophoresis. The results are shown in FIG. 7B. A band corresponding to the full-length HCV genome of ≈9,587 nt was detected. Also seen were bands corresponding to the vector (5,400 nt), a 150-nt fragment corresponding to the RNA between the T7 transcription initiation and the cleavage site of the 5′ ribozyme, and other molecular weight fragments probably representing uncleaved or prematurely terminated transcripts. A similarly expected pattern of cleavage was also observed with the pHt, which is the precursor construct of the pHr and contains the GFP sequence in place of the HCV polyprotein sequence. Further proof of the ribozymes cleaving correctly is discussed later with the RACE results.

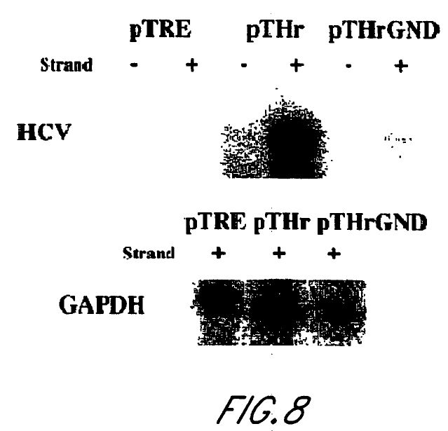

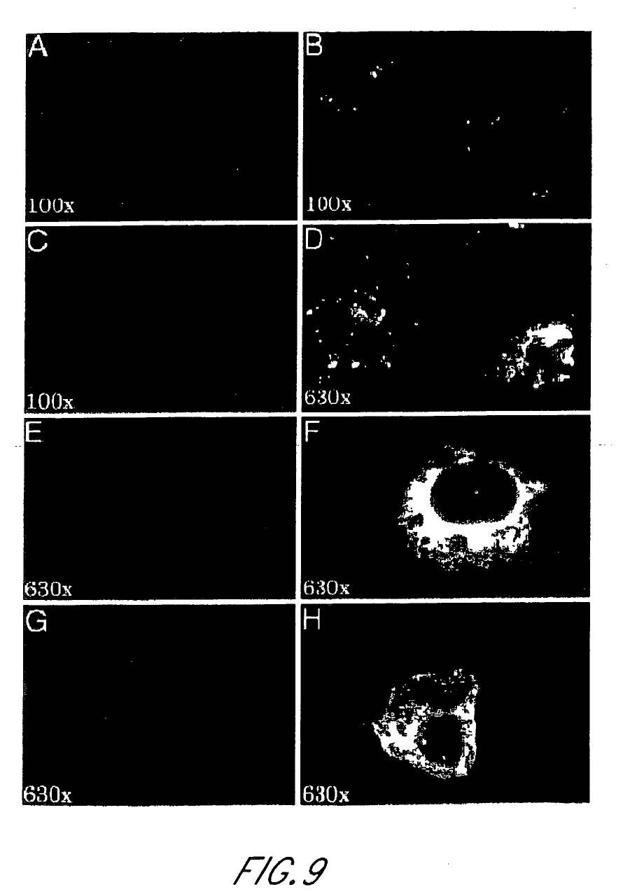

HCV RNA and Protein Production in Transfected Cells. Both positive- and negative-strand HCV RNAs were detected in cells transfected with pTHr (FIG. 8). The level of positive-strand HCV RNA was at least 10-fold higher than the level of negative-strand HCV RNA in multiple experiments. The GND mutant pTHrGND produced a small amount of positive-strand RNA but did not produce any detectable negative-strand RNA. The positive-strand RNA produced with the GND mutant was less than that produced with pTHr. No viral RNA was detected in cell lysates transfected with pTRE.

Cells transfected with pTHr or the control plasmid pTRE were analyzed by immunofluorescence with monoclonal antibodies directed against the core, E2, and NS5A. A granular cytoplasmic staining was seen with antibodies against all three proteins (FIG. 9). A time-course experiment showed peak protein expression on day 2 and a significant decrease on day 4 after transfection. The percentage of cells with fluorescence was ≈10%, despite the transfection efficiency of ≈50% with a GFP-containing plasmid. No immunofluorescence was seen in the cells transfected with pTRE.

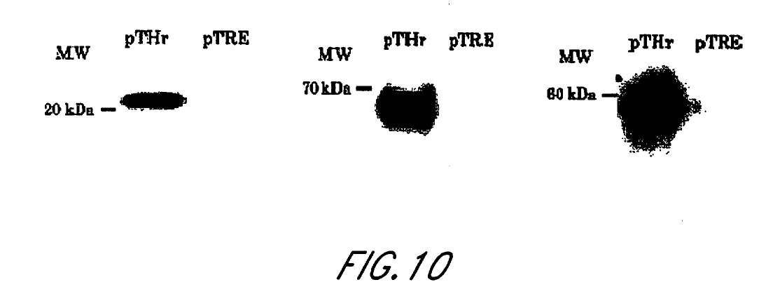

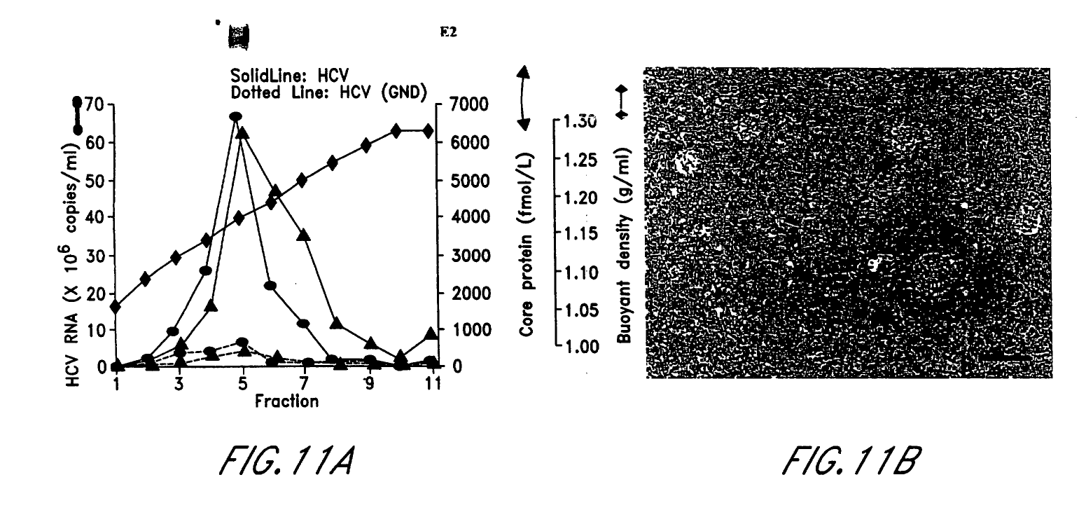

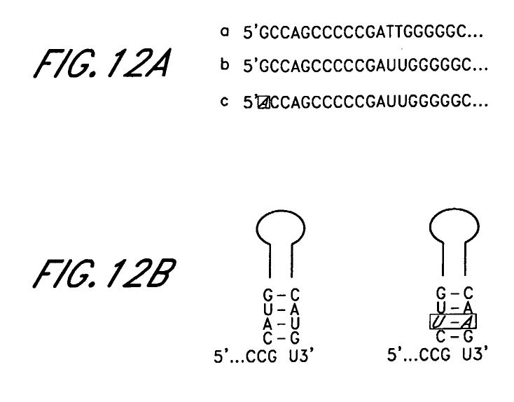

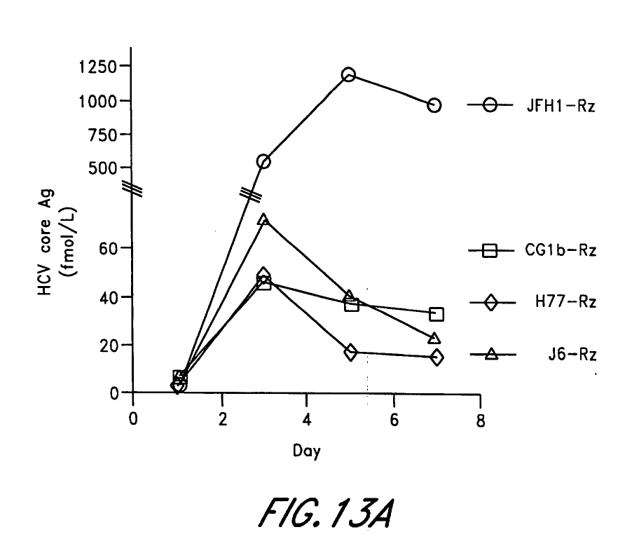

Western blot of cell lysates transfected with pTRE or pTHr showed the presence of core, E2, and NS5A in cells transfected with pTHr but not in cells transfected with pTRE (FIG. 10). As expected, viral protein was not detected in the presence of doxycycline. Furthermore, little or no HCV protein was detected in pTHrGND-transfected cells, suggesting that viral replication is required for efficient protein production in this system.

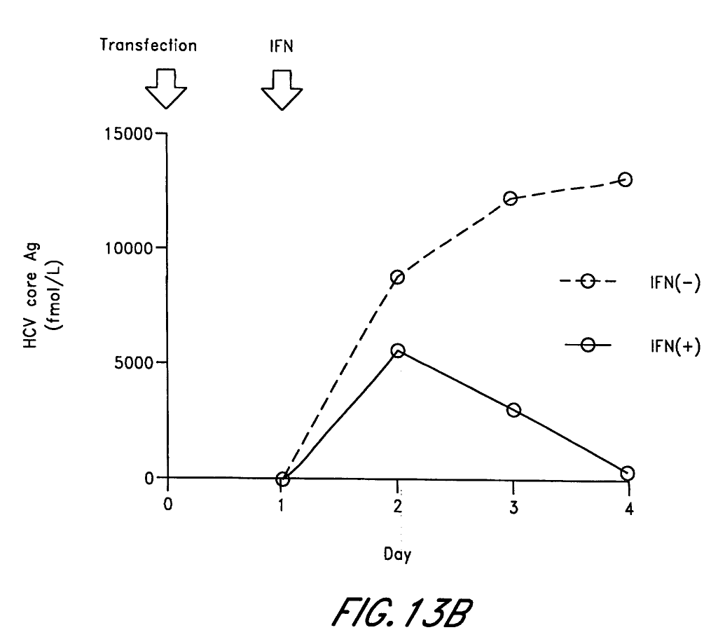

HCV Virion Production and Secretion. To assess the possibility of HCV particle production, culture medium of the pTHr- and pTHrGND-transfected cells was subjected to sucrose density gradient centrifugation. The fractions were analyzed for two HCV structural proteins, core and E2, and HCV RNA. These results are shown in FIG. 11A. In the culture medium from cells transfected with pTHr, a peak of HCV proteins and RNA coincided in fraction 5, which has the density of 1.16 g/ml. This density is consistent with the published density of free HCV virions (Kaito, M. et al. 1994 J Gen Virol 75:1755-60). Viral particles were visualized by electron microscopy only in fraction 5 (FIG. 11B). These particles are heterogeneous in appearance and have at least two sizes (≈50 and 100 nm in diameter) with the 50 nm being the major form. This heterogeneity has been described (Andre, P. et al. 2002 J Virol 76: 6919-28). Viral particles are double-shelled and appear to have spike-like projections from their surface. Shown in FIG. 11A are the results for pTHrGND-transfected cells. The HCV protein and RNA levels are at least 10-fold less than those of the pTHr-transfected cells.

Rapid Amplification of cDNA Ends (RACE). RACE was used to ensure the exact cleavage of the 5′ and 3′ ends of HCV by the ribozymes. In vitro-transcribed RNA from pHr and RNA from the culture medium of pTHr-transfected cells were analyzed by RACE. The 5′ end of the in vitro-transcribed RNA, as expected, had the same sequence as the cDNA construct (FIG. 12A). However the 3′ end of the in vitro transcript could not be amplified by RACE, possibly because of a less efficient cleavage by the 3′ ribozyme and subsequent difficulty in amplifying a heterogeneous population of the 3′ ends. Both the 5′ and 3′ ends of HCV RNA from the culture medium were successfully determined. Interestingly, a change in the most 5′ nucleotide from G to A was noted; this change has been frequently observed in HCV RNA replicons and circulating HCV RNA in infected humans (Cai, Z. et al. 2004 J. Virol. 78:3633-43). In the 3′ end, two nucleotide changes in the stem loop region were noted: U→A and A→U. These changes preserved the stem loop structure (FIG. 12B). Such changes have also been reported in HCV RNA from infected individuals (Kolykhalov, A. A. et al. 1996 J. Virol. 70:3363-71). The RNA levels in the medium of the GND-transfected cells were not adequate to perform RACE.

Discussion. Since the discovery of HCV in 1989, working with HCV has proven to be difficult, mostly because of the lack of model systems (Choo, Q. L. et al. 1989 Science 244:359-62). Each aspect of the life cycle has been difficult to reproduce in vitro. The infectious clone was developed after multiple attempts and had to be demonstrated in a chimpanzee (Kolykhalov, A. A. et al. 1997 Science 277:570-4; Yanagi, M. et al. 1997 PNAS USA 94:8738-43). Other small-animal models require complicated systems (Mercer, D. F. et al. 2001 Nat Med 7:927-33; Labonte, P. et al. 2002 J Med Virol 66:312-9). In vitro, virus obtained from infected individuals can replicate only in certain B cell lines and primary human hepatocytes but only at a low level (Shimizu, Y. K. et al. 1992 PNAS. USA 89:5477-81; Sung, V. M. et al. 2003 J. Virol. 77:2134-46). Until the development of the replicon, most model systems have been difficult to work with (Lohmann, V. et al. 1999 Science 285:110-3; Blight, K. J. et al. 2000 Science 290:1972-4). Development of virus-like particles and pseudovirus have allowed study of viral entry into the cell but do not model other aspects of the viral life cycle (Baumert, T. F. et al. 1998 J. Virol. 72:3827-36; Nam, J. H. et al. 2001 J. Virol. Methods 97:113-23; Bartosch, B. et al. 2003 PNAS USA 100:14199-204; Logvinoff, C. et al. 2004 PNAS USA 101:10149-54). Therefore, a model system with viral replication, assembly, and release is urgently needed. Furthermore, genotype 1, the most prevalent form of HCV and the most difficult to treat, was chosen for this model.

By engineering two hammerhead ribozyme sequences, one at the 5′ end and the other at the 3′ end of an infectious HCV cDNA clone, we generated a DNA expression construct for the production of HCV virions. An important initial consideration was to ensure that the ribozymes are indeed functional. This functionality was demonstrated by in vitro-translation and RACE. Transfection of this HCV-ribozyme construct into Huh7 cells demonstrated the production of structural and nonstructural proteins by immunofluorescence and Western blot. Both positive- and negative-strand RNAs could be detected intracellularly. As expected, the positive strand is much more abundant than the negative strand.

The GND mutant was constructed as a control to determine the extent of replication in this model. Evidence for replication was derived from a number of results. The simplest evidence was the presence of negative-strand viral RNA in pTHr-transfected cells and the lack of negative strand in pTHrGND-transfected cells. A >10-fold difference in the relative amounts of the positive-strand viral RNA between the wild-type and GND constructs provided additional evidence. This observation can be explained by the lack of amplification as a result of defective replication. The positive strand seen with the GND mutant was generated from transcription of the cDNA plasmid. This difference in product was also evident in the culture medium. The amounts of viral RNA and core protein on the sucrose gradients were >10-fold higher in wild-type cells than in the GND mutant-transfected cells. The final and perhaps the most interesting evidence for replication is the RACE findings. The 5′ and 3′ nucleotide changes have been described (Cai, Z. et al. 2004 J Virol 78:3633-43; Kolykhalov, A. A. et al. 1996 J Virol 70:3363-71). The G→A switch of the initial nucleotide of HCV is associated with replication in vivo and in vitro (Cai, Z. et al. 2004 J Virol 78:3633-43). A transposition from an A-T to a T-A base pairing has also been reported (Kolykhalov, A. A. et al. 1996 J Virol 70:3363-71) and represents a base pair in the putative terminal stem loop of the 3′ end of HCV. These observations provide support for the replication of viral RNA in this system.

Evidence for assembly and release was derived in a number of ways. The presence of HCV RNA in the media with the exact 5′ and 3′ ends showed that the correctly processed RNA was secreted into the culture medium. The association of viral RNA and core and E2 protein in the same fraction on the sucrose gradient with a density of 1.16 g/ml (the published density of free HCV virions) supported the interpretation that viral particles are assembled and secreted into the medium. The most compelling evidence is the visualization of particles resembling virions by electron microscopy, and these particles were visualized only in fraction 5, where viral RNA and proteins are present. It is interesting that the core protein extends into fractions 6 and 7 more than the viral RNA and E2 protein. This core reactivity might represent free core particles, although they were not seen on electron microscopy (Maillard, P. et al. 2001 J. Virol. 75:8240-50). The production and release of HCV particles is rather robust in this system, capable of achieving >10 million copies of HCV RNA per ml in the culture medium.

Although replicons using the full-length HCV genome have been developed, particles have not been described. In those replicons where sequence coding for the neomycin is included, difficulty in packaging a longer RNA molecule might be the problem. Alternatively the block could be the result of the inhibitory effects of the replicon adaptive mutations on virion assembly and release. Both possibilities are speculative. However, in the system presented here, there is no extraneous RNA and, although mutations can and do occur (see the RACE results), the source of the RNA (the cDNA) maintains a stable sequence without adaptive mutations. This difference might partially explain why particles are seen. It may also be of importance that there is a constant RNA production inside the cells being channeled directly into the appropriate cellular machinery for assembly.

This model system allows the study of events in the HCV life cycle. In addition, these particles are infectious, as described below. It should be noted that the sequence is genotype 1b. The results that would be obtained with other genotypes in this system are identical, as described below. This model represents a robust system to study the viral life cycle. This work presents an opportunity to better elucidate the biology of HCV as well as to develop therapeutic targets for the treatment of hepatitis C.

### Example 1

Plasmid Construction. The ribozymes were constructed by means of three pairs of overlapping primers that were based on a described ribozyme pair that was functional in hepatocytes (Benedict, C. M. et al. 1998 Carcinogenesis 19: 1223-30). The innermost set (5′-CGG TAC CCG GTA CCG TCG CCA GCC CCC GA (SEQ ID NO: 43) and 3′-ACG GAT CTA GAT CCG TCA CAT GAT CTG CA (SEQ ID NO: 44)) was used to amplify pHCVGFP2. The pH-CVGFP2 was derived from an infectious full-length HCV CG1b clone (Thomson, M. et al. 2001 Gastroenterology 121:1226-33) and was constructed by replacing the HCV sequences between nucleotide 709 (ClaI) and 8935 (BglII) by the sequence coding for the GFP. The middle (5′-TCC GTG AGG ACG AAA CGG TAC CCG GT (SEQ ID NO: 45) and 3′-CAC GGA CTC ATC AGG ACG GAT CTA GA (SEQ ID NO: 46)) and outermost (5′-GGC TGG CCT GAT GAG TCC GTG AGG A (SEQ ID NO: 47) and 3′-GAT CAT GTT CGT CCT CAC GGA CTC A (SEQ ID NO: 48)) sets were then added on to this sequence by PCR. This fragment was cloned into the SrfI site of pCMV-Script (Stratagene) and in turn subcloned into pcDNA3.1 (Invitrogen) by using NotI and HindIII sites to generate the pHt plasmid. pcDNA has both a CMV and a T7 promoter. The GFP was then removed, and the missing part of the HCV sequence was reinserted to generate the pHr plasmid. The pHr was used to generate the HCV-ribozyme RNA by T7 polymerase to assess the efficiency of the ribozymes. The HCV-ribozyme fragment was subcloned into pTRE2hyg+ (Clontech) under the control of a tetracycline-responsive promoter. This construct was named pTHr. In all the experiments described in this study, pTHr transfection always refers to cotransfection with pTet-Off (Clontech) expressing the tetracycline-responsive transactivator. A mutation in the GDD motif of the polymerase (GDD→GND) was introduced into this construct, and the mutated construct was then named pTHrGND. The plasmid pTREhyg2+, without any insert, was also used as a control and is hereon referred to as pTRE.

Tissue Culture and Transfection and RNase Protection Assay. A human hepatoma cell line (Huh7) was maintained at 37° C. in Dulbecco's modified Eagle's medium containing 10% FBS with 5% CO₂. Transfection was carried out by using Lipofectamine (Invitrogen) according to the manufacturer's instructions. RPA 111 ribonuclease protection assay kits (Ambion) were used according to the manufacturer's directions. The probe used was transcribed from a construct containing the core region from nucleotide 342 to nucleotide 707 of HCV CG1b strain flanked by the T3 and T7 promoters.

Immunofluorescence and Western Blot. Huh7 cells were grown on glass coverslips and transfected as described. Cells were fixed with acetone/methanol on ice at different time points after transfection. Cells were washed with PBS three times, incubated with primary antibody for 1 h, washed with PBS, incubated with secondary antibody, and washed again with PBS. Monoclonal antibodies against the core (C1) and E1 (A4) were from H. Greenberg (Stanford Medical School, Palo Alto, Calif.) (Dubuisson, J. et al. 1994 J Virol 68:6147-60). The anti-E2 monoclonal antibodies AP33 and ALP98 were from A. Patel (Medical Research Council, Glasgow, Scotland) (Triyatni, M. et al. 2002 J Virol 76:9335-44). The NS5A monoclonal antibody was obtained from J. Lau (ICN). The Cy3-labeled donkey anti-mouse IgG was obtained from Kirkegaard & Perry Laboratories. The same primary antibodies were used for Western blotting. The peroxidase-labeled goat anti-mouse IgG used as the secondary antibody was obtained from Kirkegaard & Perry Laboratories.

Sucrose Gradient Density Centrifugation. The tissue culture medium was centrifuged to remove cellular debris, and the supernatant was pelleted over a 30% sucrose cushion. The pellet was resuspended in TNC buffer (10 mM Tris.HCl, pH 7.4/1 mM CaCl₂/150 mM NaCl) with EDTA-free protease inhibitors (Roche Applied Science) and applied onto a 20-60% sucrose gradient (10.5-ml volume) in SW41 tubes (Beckman Coulter) and centrifuged at 100,000×g for 16 h at 4° C. We collected 1-ml fractions from the top of the gradient. The fractions were tested for HCV proteins and viral RNA as described below. Cryoelectron microscopy was performed by using standard techniques.

HCV RNA, Protein Quantitation, and RACE. HCV RNA level was quantitated by using the TaqMan real-time PCR method as previously described (Thomson, M. et al. 2001 Gastroenterology 121:1226-33). RNA was extracted from 100 μl of the sucrose gradient fractions or tissue culture media by using TRIzol (Invitrogen) and resuspended in 20 μl of double-filtered RNase-free water. Samples were tested in duplicate. The core protein was quantitated by using the HCV core ELISA kits, which were provided by S. Yagi (Advanced Life Technology, Saitama, Japan) and used as previously described (Tanaka, E. et al. 2000 Hepatology 32: 388-93). Samples were tested in 50- or 100-μl aliquots. RNA was extracted by using TRIzol (Invitrogen), reverse-transcribed, and amplified by RNA ligase-mediated RACE (RLM-RACE, Ambion). The 5′ and 3′ RACE procedure was performed as previously described (Cai, Z. et al. 2004 J Virol 78:3633-43).

### Example 2

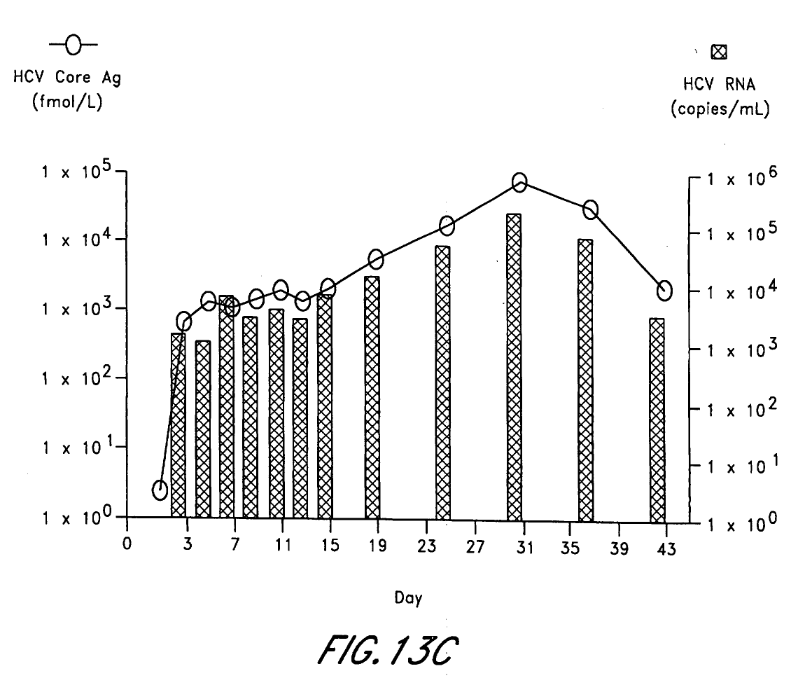

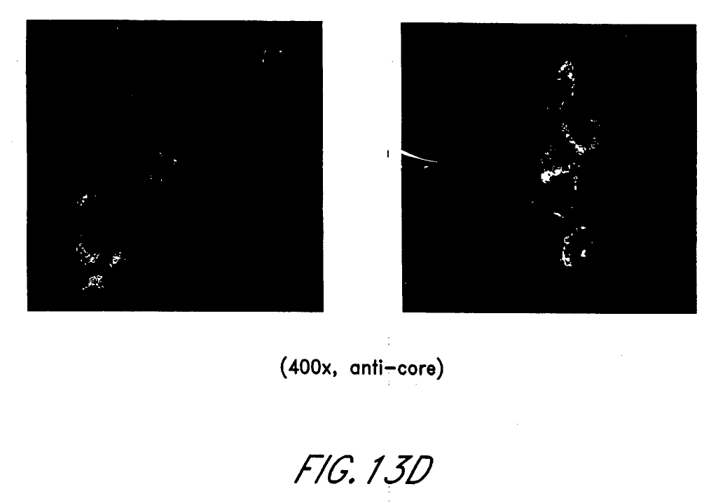

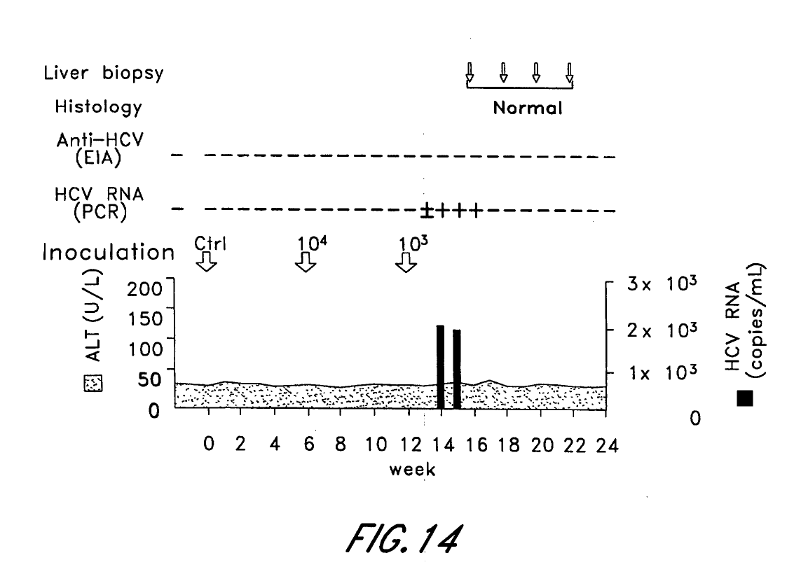

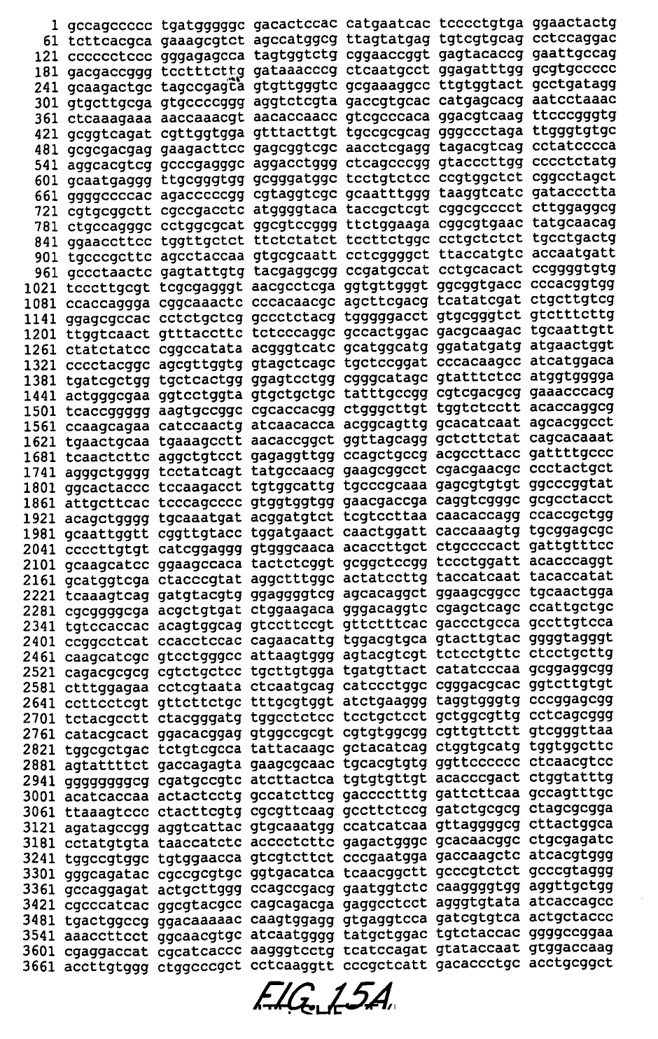

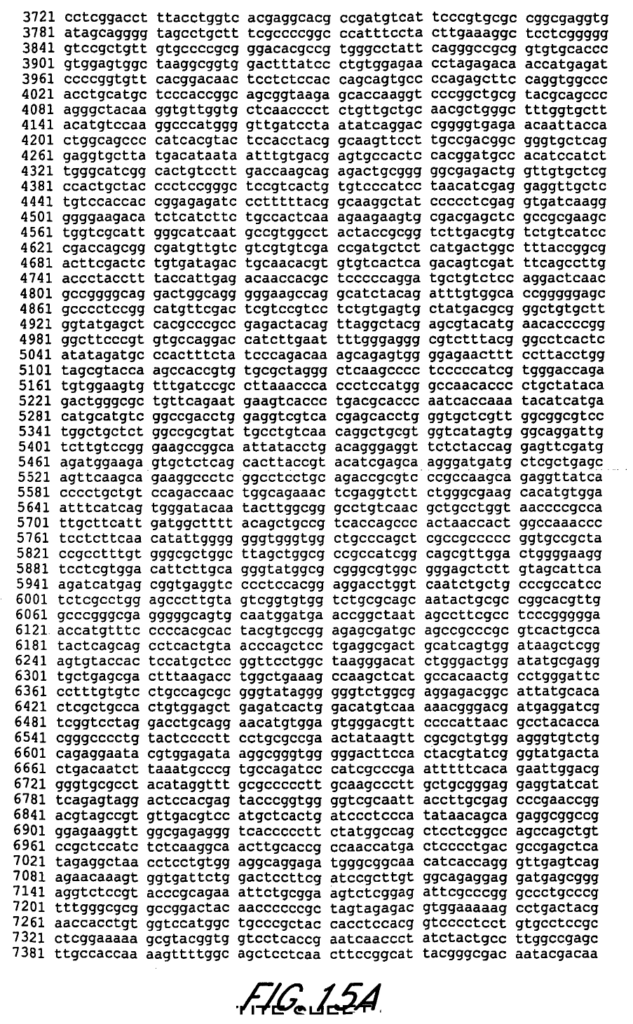

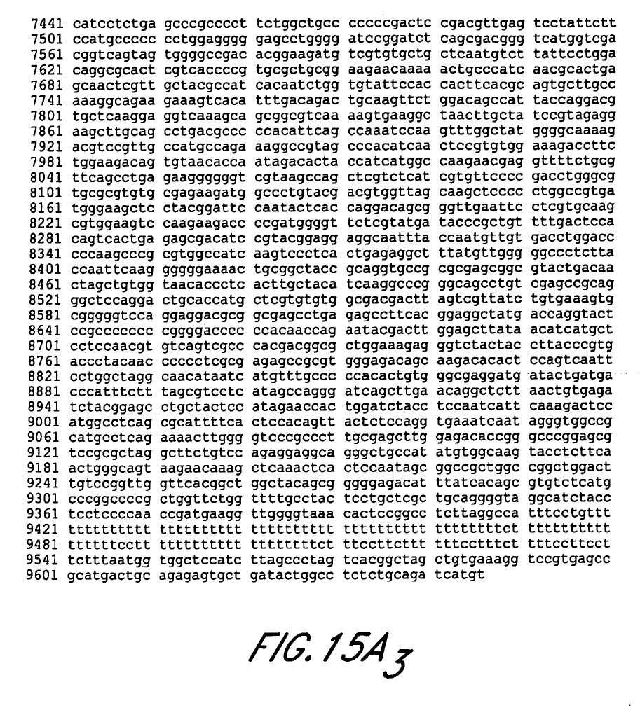

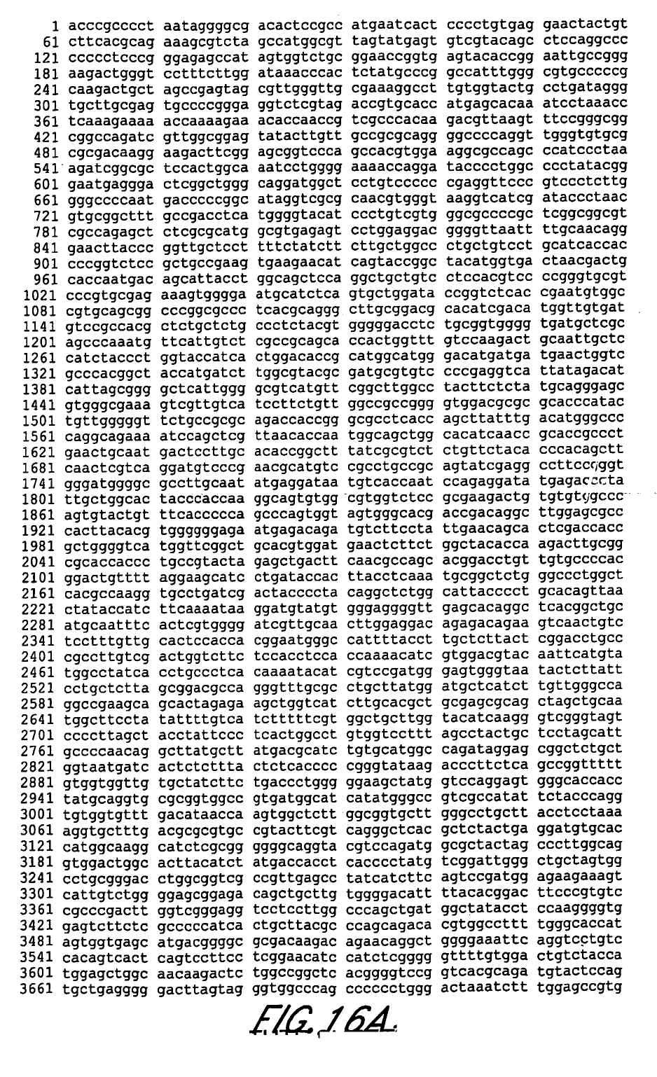

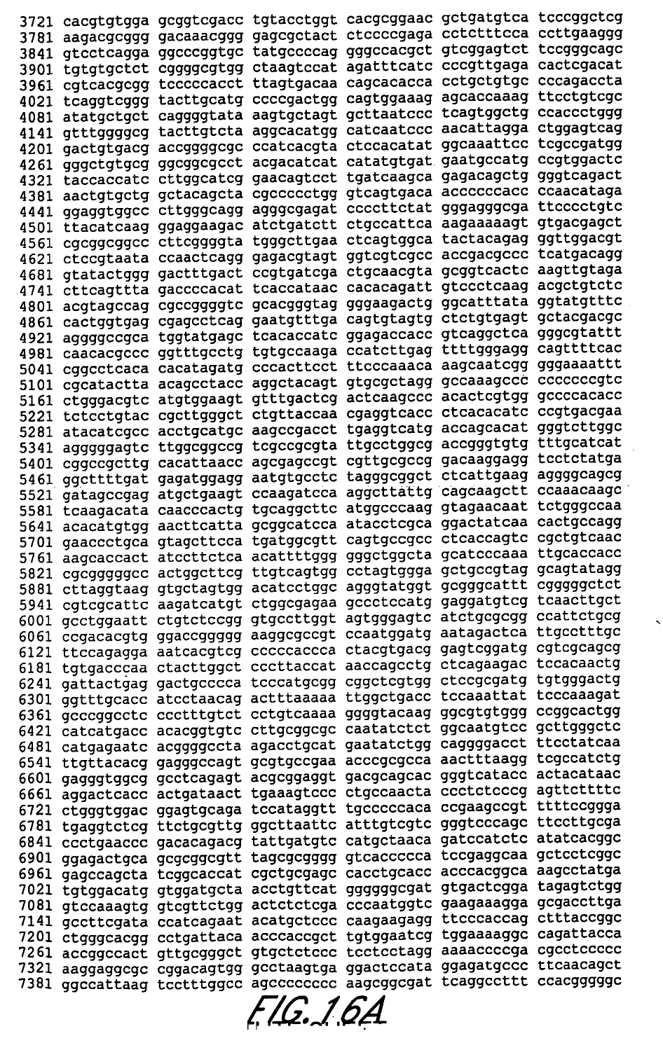

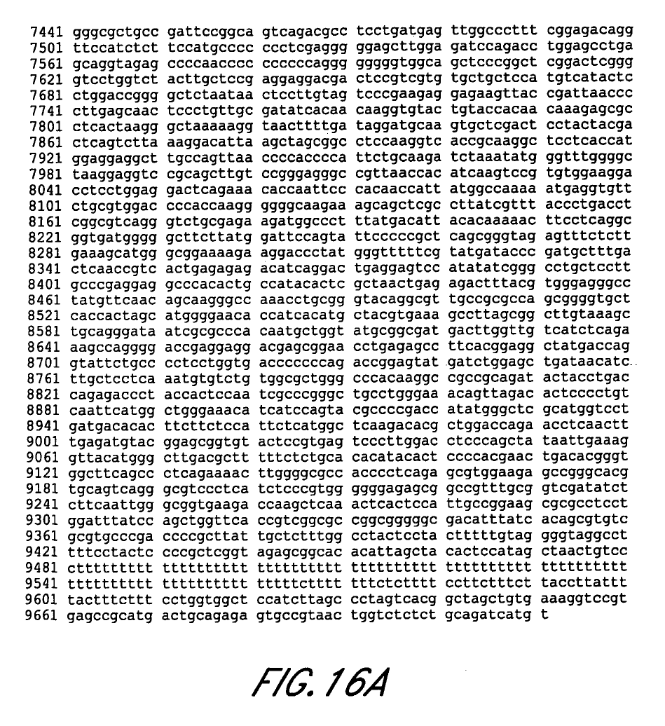

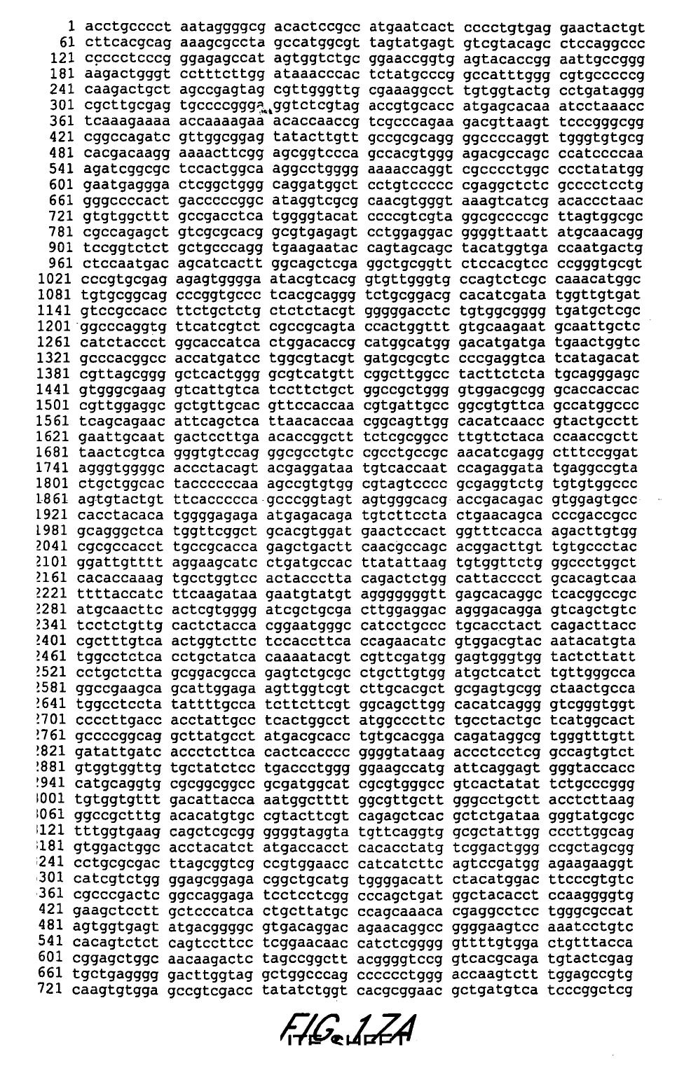

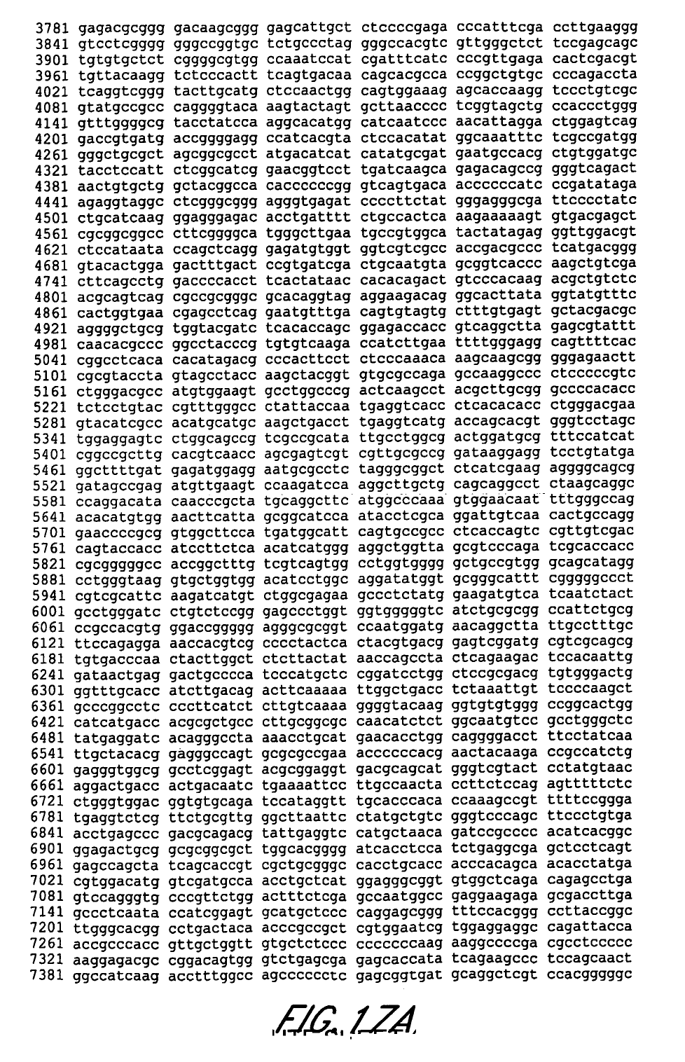

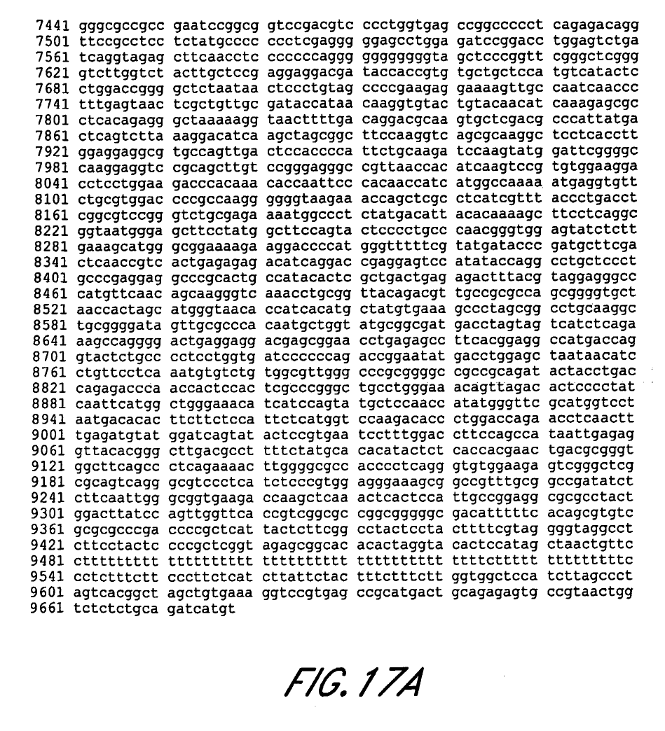

HCV can be classified into 6 genotypes. There is compelling evidence that HCV genotypic difference exists with respect to disease severity and treatment outcome. To extend our study to other HCV genotypes, we have generated HCV-ribozyme constructs expressing the genomes of other genotypes including 1a (H77, GenBank Accession No.: AF009606; SEQ ID NO: 49), 2b (J6, GenBank Accession No.: AF177036; SEQ ID NO: 50) and 2a (JFH-1, GenBank Accession No.: AB047639; SEQ ID NO: 51). We demonstrated that all these constructs, when transfected into the Huh7 cells, were capable of supporting viral replication (FIG. 13). In addition, the JFH-1 strain appeared to replicate much more efficiently than the other strains. The viral replication is also sensitive to interferon-alfa treatment (FIG. 13B). Long-term culture of the JFH-1 transfected cells demonstrated extended and high-level production of viral particles (FIG. 13C). To test the infectivity of the virus produced, culture medium from the HCV transfected cells were incubated with naïve Huh7 cells. Immunofluoresence study with anti-core antibodies demonstrated that the HCV produced by the HCV-ribozyme transfected cells were capable of infecting naïve cells (FIG. 13D). Finally to test the infectivity of the HCV produced in culture, we inoculated the HCV-containing culture medium from JFH-1 RNA transfected cells into chimpanzee. After inoculation with virus preparation containing 10³ genomes/ml, the chimpanzee became infected with active viremia (FIG. 14). To demonstrate that the virus produced in the chimpanzee was derived from the inoculated JFH-1 strain, we sequenced parts of the 5′-untranslated region (nt 128-331), the E2 hypervariable region (nt 1438-1828) and NS5B (nt 9049-9382) of the circulating viral RNA at week 4 post inoculation of the 103 dilution. The sequences were identical to those of the HCV strain used for the JFH-1 strain. This result demonstrates that the HCV virus produced in culture is also infectious in vivo.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

That which is claimed:

1. A deployable monitoring device comprising:
a self-righting housing; and
a video capturing device operably engaged with the self-righting housing and configured to capture an image external to the self-righting housing, the image comprising video data.

2. A device according to claim 1 wherein the housing further comprises a base and an opposed end disposed along an axis, the housing being configured to have a center of gravity disposed about the base so as to be self-righting along the axis such that the housing, when righted, is supported by the base.

3. A device according to claim 2 further comprising at least one stabilizer device operably engaged with the housing about the base thereof, the at least one stabilizer device extending radially outward of the base and substantially axially along an outer surface of the base, and configured to be capable of stopping rotation of the housing about the axis before the housing is righted, following deployment of the housing, so as to facilitate self-righting thereof.

4. A device according to claim 1 further comprising a transceiver module disposed within the housing and operably engaged with the video capturing device, the transceiver module being adapted to transmit the video data of the scene to a station disposed remotely from the scene, the station being configured to process the video data so as to provide a visual depiction of the scene.

5. A device according to claim 2 wherein the base further comprises a planar portion having the axis perpendicular thereto so as to facilitate stabilization of the housing about the base, upon self-righting of the housing, such that the housing is supported by the planar portion.

6. A device according to claim 5 wherein the planar portion comprises one end of the at least one stabilizer device.

7. A device according to claim 1 further comprising a power source disposed within the housing and operably engaged with the video capturing device.

8. A device according to claim 1 wherein the video capturing device comprises a video capture module configured to capture video data through a lens member in communication therewith.

9. A device according to claim 8 wherein the video capture module and the lens member are disposed within the housing and the housing is configured such that the video capture module is capable of capturing video data of the scene through the housing via the lens member.

10. A device according to claim 9 wherein the housing is at least partially translucent so as to allow the lens member to receive video data of the scene therethrough.

11. A device according to claim 8 wherein the video capture module is disposed within the housing and the lens member is at least partially disposed in an orifice defined by the housing such that the video capture module is capable of capturing video data of the scene via the lens member.

12. A device according to claim 1 wherein the video capturing device comprises at least one of a complementary metal-oxide semiconductor (CMOS) camera and a charge coupled device (CCD) camera.

13. A device according to claim 1 wherein the video capturing device is configured to be responsive to at least one of visible light and infrared light.

14. A device according to claim 1 further comprising a light source operably engaged with the housing and configured to illuminate the scene.

15. A device according to claim 1 wherein the video capturing device is configured to automatically focus on the scene.

16. A device according to claim 4 wherein the video capturing device is configured to be manually focused and is responsive to a focus command from the remotely disposed station received via the transceiver module.

17. A device according to claim 1 further comprising a motion sensor device operably engaged with the video capturing device for actuating the video capturing device to capture video data upon detection of a motion in the scene.

18. A device according to claim 4 further comprising an audio sensor operably engaged with the transceiver module and configured to capture audio data from the scene, wherein the transceiver module is further adapted to transmit the audio data from the scene to the remotely disposed station so as to provide remote audio monitoring of the scene.

19. A device according to claim 4 further comprising a chemical sensor operably engaged with the transceiver module and configured to capture chemical composition data from the scene, wherein the transceiver module is further adapted to transmit the chemical composition data from the scene to the remotely disposed station so as to provide remote chemical monitoring of the scene.

20. A device according to claim 4 further comprising a self-destruction device operably engaged with the transceiver module and configured to destroy the monitoring device, the self-destruction device being further configured to be at least one of automatically activated and manually activated in response to a destruct command from the remotely disposed station received via the transceiver module.

21. A device according to claim 4 further comprising a gimbal mechanism operably engaged between the video capturing device and the housing, the gimbal mechanism being configured to at least one of pan, tilt, and rotate the video capturing device.

22. A device according to claim 21 wherein the gimbal mechanism is configured to rotate the video capturing device about the axis.

23. A device according to claim 22 wherein the gimbal mechanism is further configured to tilt the video capturing device over a range of between about 30 degrees below a horizontal plane and about 90 degrees above the horizontal plane.

24. A device according to claim 21 wherein the gimbal mechanism is operably engaged with the transceiver module and is responsive to a movement command from the remotely disposed station received via the transceiver module.

25. A device according to claim 21 further comprising a motion sensor device operably engaged with the gimbal mechanism, the gimbal mechanism being responsive to the motion sensor device to pan and tilt the video capturing device such that video data is captured where a motion is detected in the scene.

26. A device according to claim 4 further comprising a plurality of video capturing devices operably engaging the transceiver module, each video capturing device being configured to capture video data over an angular field of view, wherein the plurality of video capturing devices are disposed within the housing and configured so as to capture video data over a 360 degree field of view about the housing.

27. A device according to claim 26 wherein each video capturing device is configured to capture video data over about a 90 degree field of view and the plurality of video capturing devices is configured to be at least one of selectively actuatable, simultaneously actuatable, and sequentially actuatable.

28. A device according to claim 4 further comprising an antenna operably engaged with the transceiver module and adapted to facilitate wireless communication between the transceiver module and the remotely disposed station.

29. A device according to claim 1 further comprising a tether operably engaged with the housing and configured to allow at least one of retrieval of the housing, movement of the housing, and positional adjustment of the housing following deployment of the monitoring device.

30. A device according to claim 1 further comprising a sound source operably engaged with the housing and configured to emit sound.

31. A device according to claim 1 further comprising an elongate member having a first end configured to be operably engaged with the housing and an opposing second end adapted to be held by an operator, the elongate member being configured to remotely support the housing, via the second end thereof, at a distance from the second end.

32. A device according to claim 1 further comprising a spatial orientation device operably engaged with the video capturing device and configured to cooperate therewith so as to associate a spatial orientation with the captured video data and thereby spatially orient the scene with respect to the video capturing device and the housing.

33. A device according to claim 32 wherein the spatial orientation device further comprises at least one of a Global Positioning System (GPS) device and a compass device.

34. A device according to claim 32 wherein the spatial orientation device is further configured to at least one of associate geodetic data regarding the housing with the captured video data and associate a compass heading of the scene, with respect to the housing, with the captured video data.

35. A device according to claim 32 wherein the spatial orientation further comprises at least one of a position, a degree heading with respect to a compass, and a compass heading.

36. A device according to claim 1 further comprising a cross-hair generator operably engaged with the video capturing device and configured to cooperate therewith so as to associate a cross-hair indicator with the captured video data to thereby orient the scene with respect to the video capturing device.

37. A device according to claim 1 further comprising a range-determining device operably engaged with the video capturing device and configured to cooperate therewith so as to associate a distance of an object within the scene, from the housing, with the captured video data.

38. A device according to claim 1 wherein the housing is configured to be one of substantially ovately-shaped and substantially spherically-shaped.

39. A method of remotely viewing an image, said method comprising:
deploying a monitoring device, comprising a video capturing device and a transceiver module operably engaged with a self-righting housing, to a remote scene; and
receiving an image of the scene external to the self-righting housing, the image being captured by the video capturing device as video data, at a station remotely disposed with respect to the housing, via the transceiver module, the video data thereby providing a remote visual depiction of the scene.

40. A method according to claim 39 wherein the housing further defines an axis, and the method further comprising stopping rotation of the housing about the axis before the housing is righted, following deployment of the housing and so as to facilitate self-righting thereof, with at least one stabilizer device operably engaged with the housing about the base thereof, the at least one stabilizer device extending radially outward of the base and substantially axially along an outer surface of the base.

41. A method according to claim 39 further comprising establishing a wireless communication link with the monitoring device via the transceiver module prior to receiving the image of the scene.

42. A method according to claim 39 wherein the monitoring device further comprises a power source operably engaging at least one of the video capturing device and the transceiver module, and the method further comprises actuating the video capturing device with the power source so as to initiate capturing of video data of the scene by the video capturing device.

43. A method according to claim 39 wherein the monitoring device further comprises a light source operably engaged with the transceiver module, and the method further comprises actuating the light source so as to illuminate the scene.

44. A method according to claim 39 wherein the video capturing device is configured to be at least one of automatically focused and manually focused in response to a focus command from the remotely disposed station received via the transceiver module, and the method further comprises focusing the video capturing device with respect to the scene.

45. A method according to claim 39 wherein the monitoring device further comprises a motion sensor operably engaged with the video capturing device, and the method further comprises actuating the video capturing device upon detection by the motion sensor of a motion in the scene so as to initiate capturing of video data of the scene by the video capturing device.

46. A method according to claim 39 wherein the monitoring device further comprises an audio sensor operably engaged with the transceiver module and configured to capture audio data from the scene, and the method further comprises receiving audio data of the scene at the remotely disposed station from the audio sensor, via the transceiver module, so as to facilitate remote aural monitoring of the scene.

47. A method according to claim 39 wherein the monitoring device further comprises a chemical sensor operably engaged with the transceiver module and configured to capture chemical composition data from the scene, and the method further comprises receiving chemical composition data of the scene at the remotely disposed station from the chemical sensor, via the transceiver module, so as to facilitate remote chemical monitoring of the scene.

48. A method according to claim 39 wherein the monitoring device further comprises a self-destruction device operably engaged with the transceiver module and configured to be at least one of automatically activated and manually activated in response to a destruct command from the remotely disposed station received via the transceiver module, and the method further comprises destroying the monitoring device by activating the self-destruction device.

49. A method according to claim 39 wherein the monitoring device further comprises a gimbal mechanism operably engaged between the video capturing device and the housing and configured to at least one of pan, tilt, and rotate the video capturing device, and the method further comprises panning and tilting the video capturing device with respect to the scene.

50. A method according to claim 49 wherein the gimbal mechanism is further configured to be manually actuated in response to a movement command from the remotely disposed station received via the transceiver module, and the method further comprises actuating the gimbal mechanism from the remotely disposed station, via the transceiver module, so as to pan and tilt the video capturing device with respect to the scene.

51. A method according to claim 49 wherein the monitoring device further comprises a motion sensor operably engaged with the gimbal mechanism, and the method further comprises actuating the gimbal mechanism so as to pan and tilt the video capturing device in response to the motion detector detecting a motion in the scene to thereby initiate capturing of video data by the video capturing device from where the motion is detected in the scene.

52. A method according to claim 39 wherein the monitoring device further comprises a plurality of video capturing devices operably engaging the transceiver module, with each video capturing device being configured to capture video data over an angular field of view, and disposed within the housing so as to capture video data over a 360 degree field of view about the housing, and the method further comprises selectively actuating one of the video capturing devices so as to capture video data of the scene within the field of view of the actuated video capturing device.

53. A method according to claim 52 further comprising substantially simultaneously actuating the plurality of video capturing devices so as to capture video data of the scene over a 360 degree field of view about the housing.

54. A method according to claim 52 further comprising sequentially actuating the video capturing devices so as to capture video data of the scene over a continuous field of view scan about the housing.

55. A method according to claim 39 wherein the monitoring device further comprises an antenna operably engaged with the transceiver module, and establishing a wireless communication link further comprises establishing a wireless communication link with the transceiver module of the monitoring device via the antenna.

56. A method according to claim 39 wherein the monitoring device further comprises a spatial orientation device operably engaged with the video capturing device, and the method further comprises associating a spatial orientation with the captured video data to thereby spatially orient the scene with respect to the video capturing device.

57. A method according to claim 56 wherein associating a spatial orientation with the captured video data further comprises determining the spatial orientation with at least one of a Global Positioning System (GPS) device and a compass device.

58. A method according to claim 56 wherein associating a spatial orientation with the captured video data further comprises associating at least one of geodetic data regarding the housing and a compass heading of the scene, with respect to the housing, with the captured video data.

59. A method according to claim 56 wherein associating a spatial orientation with the captured video data further comprises associating at least one of a position, a degree heading with respect to a compass, and a compass heading with the captured video data.

60. A method according to claim 39 further comprising associating a cross-hair indicator with the captured video data, the cross-hair indicator being provided by a cross-hair generator operably engaged with the video capturing device, to thereby orient the scene with respect to the video capturing device.

61. A method according to claim 39 further comprising associating a distance of an object within the scene, from the housing, with the captured video data, the distance being determined with a range-determining device operably engaged with the video capturing device.
